SEQUENCE LISTING (163 sequences)

SEQ ID NO: 1 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 2 - protein, 9 aa, Artificial Sequence
  Synthetic  SOX2 peptide (275-283)
SMYLPGAEV

SEQ ID NO: 3 - protein, 10 aa, Artificial Sequence
  Synthetic  SOX2 peptide (216-225)
YMNGSPTYSM

SEQ ID NO: 4 - protein, 9 aa, Artificial Sequence
  Synthetic  SOX2 peptide (306-314)
TAINGTLPL

SEQ ID NO: 5 - protein, 11 aa, Artificial Sequence
  Synthetic  SOX2 peptide (277-287)
YLPGAEVPEPA

SEQ ID NO: 6 - protein, 9 aa, Artificial Sequence
  Synthetic  SOX2 peptide (58-66)
KMAQENPKM

SEQ ID NO: 7 - protein, 10 aa, Artificial Sequence
  Synthetic  SOX2 peptide (118-127)
TLMKKDKYTL

SEQ ID NO: 8 - DNA, 25 bp, Artificial Sequence
  Synthetic hTCR_Calpha-R
cagccgcagcgtcatgagcagatta

SEQ ID NO: 9 - DNA, 27 bp, Artificial Sequence
  Synthetic hTCR_Cb1-R
ccacttccagggctgccttcagaaatc

SEQ ID NO: 10 - DNA, 27 bp, Artificial Sequence
  Synthetic hTCR_Cb2-R
tgggatggttttggagctagcctctgg

SEQ ID NO: 11 - DNA, 1818 bp, Artificial Sequence
  Synthetic SOX2 TCR#01 TCRbeta-P2A-TCRalpha, codon-optimized nucleotide
atggcttctctcctgttcttctgtggcgccttttacctgctgggaacaggctctatggatgctgatgtgacacagacccccagaaacagaatcaccaagacaggcaagcggatcatgctggagtgtagccagacaaaaggccacgaccggatgtactggtatagacaagatcctgggttgggccttcgactgatctactacagcttcgacgtgaaggacatcaacaagggcgagatctctgacggctacagcgtgagcagacaagctcaggccaagttttctctgtctctggaatctgccatccctaatcagacagccctgtacttctgtgccaccagcgagtgggatagcacagatacacagtactttggccccggcaccaggctgacagtgcttgaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggagacactgcttggcgtatcactggtgattctgtggctgcaactggctagagtgaactctcagcagggagaagaggatcctcaagctctgagcattcaggaaggcgaaaacgctaccatgaattgctcctacaagaccagcatcaacaacctgcagtggtaccggcagaatagcggaagaggactggttcacctgatcctgatcagaagcaatgaaagggagaagcacagcggcagactgagagttaccctggatacatccaagaaaagtagctctctgctgattacagcctctagagccgccgatacagccagctacttttgtgccacattaggacagggagcccagaaactggtgtttggacagggcacaagactgaccatcaatcccaacatccagaatcctgatcccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 12 - protein, 605 aa, Artificial Sequence
  Synthetic SOX2 TCR#01 TCRbeta-P2A-TCRalpha
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSEWDSTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATLGQGAQKLVFGQGTRLTINPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 114 aa, Artificial Sequence
  Synthetic SOX2 TCR#01 TCRVbeta
DADVTQTPRNRITKTGKRIMLECSQTKGHDRMYWYRQDPGLGLRLIYYSFDVKDINKGEISDGYSVSRQAQAKFSLSLESAIPNQTALYFCATSEWDSTDTQYFGPGTRLTVLE

SEQ ID NO: 14 - protein, 5 aa, Artificial Sequence
  Synthetic SOX2 TCR#01 CDR1beta
KGHDR

SEQ ID NO: 15 - protein, 6 aa, Artificial Sequence
  Synthetic SOX2 TCR#01 CDR2beta
SFDVKD

SEQ ID NO: 16 - protein, 12 aa, Artificial Sequence
  Synthetic SOX2 TCR#01 CDR3beta
ATSEWDSTDTQY

SEQ ID NO: 17 - protein, 14 aa, Artificial Sequence
  Synthetic SOX2 TCR#01 CDR3beta-junction
CATSEWDSTDTQYF

SEQ ID NO: 18 - protein, 112 aa, Artificial Sequence
  Synthetic SOX2 TCR#01 TCRValpha
SQQGEEDPQALSIQEGENATMNCSYKTSINNLQWYRQNSGRGLVHLILIRSNEREKHSGRLRVTLDTSKKSSSLLITASRAADTASYFCATLGQGAQKLVFGQGTRLTINPN

SEQ ID NO: 19 - protein, 5 aa, Artificial Sequence
  Synthetic SOX2 TCR#01 CDR1alpha
TSINN

SEQ ID NO: 20 - protein, 7 aa, Artificial Sequence
  Synthetic SOX2 TCR#01 CDR2alpha
IRSNERE

SEQ ID NO: 21 - protein, 11 aa, Artificial Sequence
  Synthetic SOX2 TCR#01 CDR3alpha
ATLGQGAQKLV

SEQ ID NO: 22 - protein, 13 aa, Artificial Sequence
  Synthetic SOX2 TCR#01 CDR3alpha-junction
CATLGQGAQKLVF

SEQ ID NO: 23 - DNA, 1830 bp, Artificial Sequence
  Synthetic SOX2 TCR#02 TCRbeta-P2A-TCRalpha, codon-optimized nucleotide
atgggcatcagactcctttgcagggtggccttttgctttctggctgtgggattagtggacgtgaaggttacccagtccagtagataccttgtcaagagaaccggcgagaaggtgttcctggaatgtgtgcaggacatggatcacgagaatatgttctggtacagacaggaccctggactgggactgagactgatctacttcagctacgacgtgaagatgaaggagaagggcgatatccctgagggctacagcgtgagcagagagaaaaaggagaggttcagcctgattctggaaagcgccagcacaaatcaaacatctatgtatttatgcgcctcttctctgattctggccggaagaaatacaggcgagctgttttttggcgagggctctagactgaccgtgcttgaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatgaagaagcacctgaccacgttcctggtgattctgtggctgtacttctacaggggcaacggcaagaatcaggttgagcagtctcctcagtccctgattattcttgagggcaagaattgtaccctgcagtgcaactacacagtgagccctttcagcaacctgaggtggtacaagcaggatacaggaagaggacctgtgagtttgacaatcatgaccttcagcgagaacaccaagagcaacggcagatatacagccaccctggatgccgacacaaagcagtcttctctgcacattacagcctctcagctgagcgattctgccagctacatctgcgtggtgagagacagcaattaccagctgatttggggagctggcacaaagctgatcatcaagcccgacatccagaatcccgatcctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 24 - protein, 609 aa, Artificial Sequence
  Synthetic SOX2 TCR#02 TCRbeta-P2A-TCRalpha,
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLILAGRNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVRDSNYQLIWGAGTKLIIKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 117 aa, Artificial Sequence
  Synthetic SOX2 TCR#02 TCRVbeta
DVKVTQSSRYLVKRTGEKVFLECVQDMDHENMFWYRQDPGLGLRLIYFSYDVKMKEKGDIPEGYSVSREKKERFSLILESASTNQTSMYLCASSLILAGRNTGELFFGEGSRLTVLE

SEQ ID NO: 26 - protein, 5 aa, Artificial Sequence
  Synthetic SOX2 TCR#02 CDR1beta
MDHEN

SEQ ID NO: 27 - protein, 6 aa, Artificial Sequence
  Synthetic SOX2 TCR#02 CDR2beta
SYDVKM

SEQ ID NO: 28 - protein, 15 aa, Artificial Sequence
  Synthetic SOX2 TCR#02 CDR3beta
ASSLILAGRNTGELF

SEQ ID NO: 29 - protein, 17 aa, Artificial Sequence
  Synthetic SOX2 TCR#02 CDR3beta-junction
CASSLILAGRNTGELFF

SEQ ID NO: 30 - protein, 112 aa, Artificial Sequence
  Synthetic SOX2 TCR#02 TCRValpha
KNQVEQSPQSLIILEGKNCTLQCNYTVSPFSNLRWYKQDTGRGPVSLTIMTFSENTKSNGRYTATLDADTKQSSLHITASQLSDSASYICVVRDSNYQLIWGAGTKLIIKPD

SEQ ID NO: 31 - protein, 6 aa, Artificial Sequence
  Synthetic SOX2 TCR#02 CDR1alpha
VSPFSN

SEQ ID NO: 32 - protein, 7 aa, Artificial Sequence
  Synthetic SOX2 TCR#02 CDR2alpha
MTFSENT

SEQ ID NO: 33 - protein, 10 aa, Artificial Sequence
  Synthetic SOX2 TCR#02 CDR3alpha
VVRDSNYQLI

SEQ ID NO: 34 - protein, 12 aa, Artificial Sequence
  Synthetic SOX2 TCR#02 CDR3alpha-junction
CVVRDSNYQLIW

SEQ ID NO: 35 - DNA, 1827 bp, Artificial Sequence
  Synthetic SOX2 TCR#04 TCRbeta-P2A-TCRalpha, codon-optimized nucleotide
atgggacctggactgttatgttgggttctgctgtgtctgcttggagctggacctgttgatgctggagttacacagagccctacacacctgatcaagacaagaggacagcaggtgaccctgagatgtagccctatttctggccacaagagcgtgagctggtatcagcaggttttaggacagggaccccagttcatcttccagtactacgagaaggaggagagaggcagaggcaatttccctgacagatttagcgcccggcagttccccaattatagctctgagctgaacgtgaatgccctgctgcttggcgattctgctctgtacctgtgtgcctcttctctgtttatcagcctgatgctggatacccagtacttcggccctggcacaagactgacagtgctggaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggtgaagatccggcagttcctcctggctattctgtggctgcaactgtcttgtgtgtcagcagccaagaatgaagtggagcagtcccctcagaacctgaccgcacaggaaggcgagtttatcaccatcaactgcagctactccgtaggcataagcgccctgcattggctccagcagcaccctggaggaggaattgtgtctctgtttatgctgagcagcggaaaaaaaaagcacggccggctgattgccaccatcaacatccaggagaagcatagcagcctacacattacagcgtctcatcccagggattctgccgtgtacatctgtgccacactgacacaaggaggaagcgaaaaactggtgtttggcaagggcacaaagctgaccgtgaatccctacatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 36 - protein, 608 aa, Artificial Sequence
  Synthetic SOX2 TCR#04 TCRbeta-P2A-TCRalpha
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSLFISLMLDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICATLTQGGSEKLVFGKGTKLTVNPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 37 - protein, 116 aa, Artificial Sequence
  Synthetic SOX2 TCR#04 TCRVbeta
DAGVTQSPTHLIKTRGQQVTLRCSPISGHKSVSWYQQVLGQGPQFIFQYYEKEERGRGNFPDRFSARQFPNYSSELNVNALLLGDSALYLCASSLFISLMLDTQYFGPGTRLTVLE

SEQ ID NO: 38 - protein, 5 aa, Artificial Sequence
  Synthetic SOX2 TCR#04 CDR1beta
SGHKS

SEQ ID NO: 39 - protein, 6 aa, Artificial Sequence
  Synthetic SOX2 TCR#04 CDR2beta
YYEKEE

SEQ ID NO: 40 - protein, 14 aa, Artificial Sequence
  Synthetic SOX2 TCR#04 CDR3beta
ASSLFISLMLDTQY

SEQ ID NO: 41 - protein, 16 aa, Artificial Sequence
  Synthetic SOX2 TCR#04 CDR3beta-junction
CASSLFISLMLDTQYF

SEQ ID NO: 42 - protein, 111 aa, Artificial Sequence
  Synthetic SOX2 TCR#04 TCRValpha
KNEVEQSPQNLTAQEGEFITINCSYSVGISALHWLQQHPGGGIVSLFMLSSGKKKHGRLIATINIQEKHSSLHITASHPRDSAVYICATLTQGGSEKLVFGKGTKLTVNPY

SEQ ID NO: 43 - protein, 5 aa, Artificial Sequence
  Synthetic SOX2 TCR#04 CDR1alpha
VGISA

SEQ ID NO: 44 - protein, 5 aa, Artificial Sequence
  Synthetic SOX2 TCR#04 CDR2alpha
LSSGK

SEQ ID NO: 45 - protein, 12 aa, Artificial Sequence
  Synthetic SOX2 TCR#04 CDR3alpha
ATLTQGGSEKLV

SEQ ID NO: 46 - protein, 14 aa, Artificial Sequence
  Synthetic SOX2 TCR#04 CDR3alpha-junction
CATLTQGGSEKLVF

SEQ ID NO: 47 - DNA, 1833 bp, Artificial Sequence
  Synthetic SOX2 TCR#05 TCRbeta-P2A-TCRalpha, codon-optimized nucleotide
atgggcatcagactcctttgcagggtggccttttgctttctggctgtgggattagtggacgtgaaggttacccagtccagcagatacctggttaagagaaccggcgagaaggtgttcctggaatgtgtgcaggacatggatcacgaaaatatgttttggtacagacaggaccctggactgggactgagactgatctacttcagctacgacgtgaagatgaaggagaagggcgatatccctgagggctacagcgtgagcagagagaaaaaggagaggttcagcctgattctggaaagcgccagcacaaatcaaacatctatgtacctttgcgcctctagacctgaaatgggcggaagaggaaatcagcctcagcattttggcgatggcacaagactgagcatcctggaggatctgaacaaggtgttccccccagaggtggccgtgttcgagccttctgaggccgagatctcccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccacaatcctgtacgagatcctgctgggcaaggccaccctgtacgccgtgctggtgtctgccctggtgctgatggccatggtcaagcggaaggacttcggttccggagccacgaacttctctctgttaaagcaagcaggagacgtggaagaaaaccccggtcccatgagcctgtcttctctgctgaaggtggtgactgctagcctctggttaggacctggaattgcccagaagatcacccagacacaacctggcatgtttgtgcaggagaaagaggctgtgaccctggattgcacctacgataccagcgatccttcttacggccttttctggtacaaacagcctagctccggcgagatgatcttcctgatctaccagggcagctacgaccagcagaacgcaaccgagggtagatatagcctgaattttcagaaggcaagaaagtcagccaacctggtgatctctgcctctcaactgggcgattctgccatgtacttttgtgccatgagagagggcatgggcttccagaaactggtgtttggcacaggcacaaggctgctggtttctcccaatatccagaatcctgaccctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 48 - protein, 610 aa, Artificial Sequence
  Synthetic SOX2 TCR#05 TCRbeta-P2A-TCRalpha
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRPEMGGRGNQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGMGFQKLVFGTGTRLLVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 49 - protein, 117 aa, Artificial Sequence
  Synthetic SOX2 TCR#05 TCRVbeta
DVKVTQSSRYLVKRTGEKVFLECVQDMDHENMFWYRQDPGLGLRLIYFSYDVKMKEKGDIPEGYSVSREKKERFSLILESASTNQTSMYLCASRPEMGGRGNQPQHFGDGTRLSILE

SEQ ID NO: 50 - protein, 5 aa, Artificial Sequence
  Synthetic SOX2 TCR#05 CDR1beta
MDHEN

SEQ ID NO: 51 - protein, 6 aa, Artificial Sequence
  Synthetic SOX2 TCR#05 CDR2beta
SYDVKM

SEQ ID NO: 52 - protein, 15 aa, Artificial Sequence
  Synthetic SOX2 TCR#05 CDR3beta
ASRPEMGGRGNQPQH

SEQ ID NO: 53 - protein, 17 aa, Artificial Sequence
  Synthetic SOX2 TCR#05 CDR3beta-junction
CASRPEMGGRGNQPQHF

SEQ ID NO: 54 - protein, 116 aa, Artificial Sequence
  Synthetic SOX2 TCR#05 TCRValpha
AQKITQTQPGMFVQEKEAVTLDCTYDTSDPSYGLFWYKQPSSGEMIFLIYQGSYDQQNATEGRYSLNFQKARKSANLVISASQLGDSAMYFCAMREGMGFQKLVFGTGTRLLVSPN

SEQ ID NO: 55 - protein, 7 aa, Artificial Sequence
  Synthetic SOX2 TCR#05 CDR1alpha
TSDPSYG

SEQ ID NO: 56 - protein, 8 aa, Artificial Sequence
  Synthetic SOX2 TCR#05 CDR2alpha
QGSYDQQN

SEQ ID NO: 57 - protein, 12 aa, Artificial Sequence
  Synthetic SOX2 TCR#05 CDR3alpha
AMREGMGFQKLV

SEQ ID NO: 58 - protein, 14 aa, Artificial Sequence
  Synthetic SOX2 TCR#05 CDR3alpha-junction
CAMREGMGFQKLVF

SEQ ID NO: 59 - DNA, 1523 bp, Artificial Sequence
  Synthetic SOX2 TCR#06 TCRbeta-P2A-TCRalpha, codon-optimized nucleotide
atgcttttactccttctcttgctcggacctgctggatctggattaggagctgttgtgtctcagcacccttcttgggtgatctgtaaaagcggcacaagcgtgaaaattgagtgcaggagcctggattttcaggccaccaccatgttctggtataggcagttccccaagcagtctctgatgctgatggccacctctaatgagggctctaaggccacatatgagcagggcgtagagaaggacaagttcctgatcaaccacgcctctctgaccctgtctaccctgacagttacatctgcccaccctgaggatagcagcttttacatctgtagcgccagaggcaagacaagccctagacctgaaacccagtacttcggacctggcacaagactgttagttctggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggagtcttcattgggtggtgtgctgctgattctgtggctgcaggtagattgggtcaagagccagaagatcgagcagaattctgaagccctgaacatccaggagggcaagacagctacactgacctgcaactacaccaactactctcctgcttacctgcagtggtacagacaggatcctggcagaggacctgtgtttctgctgctgattagagaaaacgaaaaggagaaaagaaaagagagactgaaggttaccttcgacacaaccctgaagcagagcctgtttcacattacagccagccaacccgccgattctgccacatacctgtgtgctctgggacacaagctgacatttggaggaggaacaagagttctggtgaagcccaacatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccga

SEQ ID NO: 60 - protein, 603 aa, Artificial Sequence
  Synthetic SOX2 TCR#06 TCRbeta-P2A-TCRalpha
MLLLLLLLGPAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARGKTSPRPETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMESSLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALGHKLTFGGGTRVLVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 61 - protein, 119 aa, Artificial Sequence
  Synthetic SOX2 TCR#06 TCRVbeta
GAVVSQHPSWVICKSGTSVKIECRSLDFQATTMFWYRQFPKQSLMLMATSNEGSKATYEQGVEKDKFLINHASLTLSTLTVTSAHPEDSSFYICSARGKTSPRPETQYFGPGTRLLVLE

SEQ ID NO: 62 - protein, 6 aa, Artificial Sequence
  Synthetic SOX2 TCR#06 CDR1beta
DFQATT

SEQ ID NO: 63 - protein, 7 aa, Artificial Sequence
  Synthetic SOX2 TCR#06 CDR2beta
SNEGSKA

SEQ ID NO: 64 - protein, 14 aa, Artificial Sequence
  Synthetic SOX2 TCR#06 CDR3beta
SARGKTSPRPETQY

SEQ ID NO: 65 - protein, 16 aa, Artificial Sequence
  Synthetic SOX2 TCR#06 CDR3beta-junction
CSARGKTSPRPETQYF

SEQ ID NO: 66 - protein, 109 aa, Artificial Sequence
  Synthetic SOX2 TCR#06 TCRValpha
SQKIEQNSEALNIQEGKTATLTCNYTNYSPAYLQWYRQDPGRGPVFLLLIRENEKEKRKERLKVTFDTTLKQSLFHITASQPADSATYLCALGHKLTFGGGTRVLVKPN

SEQ ID NO: 67 - protein, 6 aa, Artificial Sequence
  Synthetic SOX2 TCR#06 CDR1alpha
NYSPAY

SEQ ID NO: 68 - protein, 7 aa, Artificial Sequence
  Synthetic SOX2 TCR#06 CDR2alpha
IRENEKE

SEQ ID NO: 69 - protein, 7 aa, Artificial Sequence
  Synthetic SOX2 TCR#06 CDR3alpha
ALGHKLT

SEQ ID NO: 70 - protein, 9 aa, Artificial Sequence
  Synthetic SOX2 TCR#06 CDR3alpha-junction
CALGHKLTF

SEQ ID NO: 71 - DNA, 1830 bp, Artificial Sequence
  Synthetic SOX2 TCR#07 TCRbeta-P2A-TCRalpha codon-optimized nucleotide
atgggcacaagactccttttctgggtcgccttttgtctgcttggagccgatcataccggcgccggcgtgagccagtctcccagcaataaggtcaccgagaagggcaaagatgtggagctgagatgcgaccctatttctggccataccgccctgtactggtacagacaatctctgggacaggggctagagttcctgatctactttcagggcaatagcgcccccgacaaatctggacttcctagcgacaggttttctgccgagagaacaggaggatctgtttctaccctgaccatccagagaacacagcaggaggattctgccgtgtacctgtgtgccagcagaagaacaggatggagctacaatgagcagttctttggccctggaaccagactgacagtgctggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatgatcagcctgagagtcctgctcgtgattctgtggctgcaactgtcttgggtttggagccagagaaaagaagtggagcaggatcctggcccctttaatgtgcctgaaggagctacagtggccttcaactgcacatacagcaacagcgcctcacagagcttcttctggtacagacaggactgcaggaaggagcctaagctgctgatgtctgtgtatagcagcggcaatgaggatggcagatttacagcccagctgaaccgggcttctcagtacatctctctgctgatcagggatagcaagctgagcgattctgctacatacctctgcgtggtgtacgtggagtctggaggcagctatatccctacattcggcagaggaacaagcctgattgtgcacccctatatccagaatcccgaccctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 72 - protein, 609 aa, Artificial Sequence
  Synthetic SOX2 TCR#07 TCRbeta-P2A-TCRalpha
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASRRTGWSYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVYVESGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 73 - protein, 116 aa, Artificial Sequence
  Synthetic SOX2 TCR#07 TCRVbeta
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASRRTGWSYNEQFFGPGTRLTVLE

SEQ ID NO: 74 - protein, 5 aa, Artificial Sequence
  Synthetic SOX2 TCR#07 CDR1beta
SGHTA

SEQ ID NO: 75 - protein, 6 aa, Artificial Sequence
  Synthetic SOX2 TCR#07 CDR2beta
FQGNSA

SEQ ID NO: 76 - protein, 13 aa, Artificial Sequence
  Synthetic SOX2 TCR#07 CDR3beta
ASRRTGWSYNEQF

SEQ ID NO: 77 - protein, 15 aa, Artificial Sequence
  Synthetic SOX2 TCR#07 CDR3beta-junction
CASRRTGWSYNEQFF

SEQ ID NO: 78 - protein, 113 aa, Artificial Sequence
  Synthetic SOX2 TCR#07 TCRValpha
RKEVEQDPGPFNVPEGATVAFNCTYSNSASQSFFWYRQDCRKEPKLLMSVYSSGNEDGRFTAQLNRASQYISLLIRDSKLSDSATYLCVVYVESGGSYIPTFGRGTSLIVHPY

SEQ ID NO: 79 - protein, 6 aa, Artificial Sequence
  Synthetic SOX2 TCR#07 CDR1alpha
NSASQS

SEQ ID NO: 80 - protein, 6 aa, Artificial Sequence
  Synthetic SOX2 TCR#07 CDR2alpha
VYSSGN

SEQ ID NO: 81 - protein, 13 aa, Artificial Sequence
  Synthetic SOX2 TCR#07 CDR3alpha
VVYVESGGSYIPT

SEQ ID NO: 82 - protein, 15 aa, Artificial Sequence
  Synthetic SOX2 TCR#07 CDR3alpha-junction
CVVYVESGGSYIPTF

SEQ ID NO: 83 - DNA, 1833 bp, Artificial Sequence
  Synthetic SOX2 TCR#09 TCRbeta-P2A-TCRalpha, codon-optimized nucleotide
atggatacctggctcgtctgttgggccatcttttctctgctgaaagctggcctgacagaacctgaggttacacagacacctagccaccaggtgactcagatgggacaggaagtgatcctgagatgtgtgcccatcagcaatcacctgtacttttattggtacaggcagattctgggccagaaggtcgaattcctggtgagcttttacaataacgagattagcgagaaatccgaaatcttcgacgatcagttctcagtggagagacctgatggcagcaattttaccctgaagattcgcagcacaaagctggaggatagcgccatgtacttttgtgcgagttctagacgaggcgggcctattgaaacccagtattttggccctggaacaagactgctggtccttgaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatgatgaagagcctgcgagtcctcctggtgattctgtggctgcaactttcttgggtgtggtctcagcagaaagaagtggagcaggatcctggacctctgtctgtgcctgaaggagctattgtgagcctcaattgtacttacagcaatagcgccttccagtacttcatgtggtaccggcagtacagcaggaagggccctgaactgctcatgtatacctattctagcggcaataaggaggatggcaggtttacagcccaggtggacaagagctccaagtatataagtttattcatcagagatagccagcctagcgattctgccacctacctgtgtgccatgtctgccgacaatgcaggtaatatgctgacatttggaggcggaacaaggctgatggtgaaaccccacatccagaaccccgaccccgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 84 - protein, 610 aa, Artificial Sequence
  Synthetic SOX2 TCR#09 TCRbeta-P2A-TCRalpha
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSRRGGPIETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSADNAGNMLTFGGGTRLMVKPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 85 - protein, 116 aa, Artificial Sequence
  Synthetic SOX2 TCR#09 TCRVbeta
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSRRGGPIETQYFGPGTRLLVLE

SEQ ID NO: 86 - protein, 5 aa, Artificial Sequence
  Synthetic SOX2 TCR#09 CDR1beta
SNHLY

SEQ ID NO: 87 - protein, 6 aa, Artificial Sequence
  Synthetic SOX2 TCR#09 CDR2beta
FYNNEI

SEQ ID NO: 88 - protein, 13 aa, Artificial Sequence
  Synthetic SOX2 TCR#09 CR3beta
ASSRRGGPIETQY

SEQ ID NO: 89 - protein, 15 aa, Artificial Sequence
  Synthetic SOX2 TCR#09 CR3beta-junction
CASSRRGGPIETQYF

SEQ ID NO: 90 - protein, 113 aa, Artificial Sequence
  Synthetic SOX2 TCR#09 TCRValpha
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSADNAGNMLTFGGGTRLMVKPH

SEQ ID NO: 91 - protein, 6 aa, Artificial Sequence
  Synthetic SOX2 TCR#09 CDR1alpha
NSAFQY

SEQ ID NO: 92 - protein, 6 aa, Artificial Sequence
  Synthetic SOX2 TCR#09 CDR2alpha
TYSSGN

SEQ ID NO: 93 - protein, 12 aa, Artificial Sequence
  Synthetic SOX2 TCR#09 CDR3alpha
AMSADNAGNMLT

SEQ ID NO: 94 - protein, 14 aa, Artificial Sequence
  Synthetic SOX2 TCR#09 CDR3alpha-junction
CAMSADNAGNMLTF

SEQ ID NO: 95 - DNA, 1809 bp, Artificial Sequence
  Synthetic SOX2 TCR#10 TCRbeta-P2A-TCRalpha, codon-optimized nucleotide
atgaccattcgactgctgtgctacatgggcttctacttcctgggagctggactgatggaagccgacatctaccaaacacccagatacctggtgatcggaacaggcaagaagatcaccctggagtgtagccagacaatgggacacgacaagatgtactggtaccagcaggatcctggaatggaactgcacctgatccactacagctatggcgtgaacagcacagagaaaggcgatctgagcagcgagtctacagtgagcagaatcagaaccgagcacttccctctgacactggaatctgctagacctagccacacaagccagtacctgtgtgctagctctagaggacctgaggccttttttggacagggaacaagactgacagtggtggaggatctgaacaaggtgttccccccagaggtggccgtgttcgagccttctgaggccgagatctcccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccacaatcctgtacgagatcctgctgggcaaggccaccctgtacgccgtgctggtgtctgccctggtgctgatggccatggtcaagcggaaggacttcggttccggagccacgaacttctctctgttaaagcaagcaggagacgtggaagaaaaccccggtcccatgaagacctttgccggattctcattcctatttctctggctgcagctggattgtatgagcagaggcgaagatgtggaacagagcctgtttctgagcgtgagagagggcgatagctctgtgatcaattgcacatacacggatagcagcagcacctacctgtactggtataaacaggagccgggcgcgggcctgcaactgctgacatacatcttcagcaatatggatatgaagcaggaccagaggttgaccgtcttactcaataagaaagacaagcacctgtctctgagaatcgctgacactcagactggcgatagcgccatctacttctgtgctggacctcttggcggatctcagggaaatcttatcttcggtaagggcacaaagctgagcgtgaagcccaatatacaaaacccagaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 96 - protein, 602 aa, Artificial Sequence
  Synthetic SOX2 TCR#10 TCRbeta-P2A-TCRalpha
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSRGPEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAGPLGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 97 - protein, 111 aa, Artificial Sequence
  Synthetic SOX2 TCR#10 TCRVbeta
EADIYQTPRYLVIGTGKKITLECSQTMGHDKMYWYQQDPGMELHLIHYSYGVNSTEKGDLSSESTVSRIRTEHFPLTLESARPSHTSQYLCASSRGPEAFFGQGTRLTVVE

SEQ ID NO: 98 - protein, 5 aa, Artificial Sequence
  Synthetic SOX2 TCR#10 CDR1beta
MGHDK

SEQ ID NO: 99 - protein, 6 aa, Artificial Sequence
  Synthetic SOX2 TCR#10 CDR2beta
SYGVNS

SEQ ID NO: 100 - protein, 9 aa, Artificial Sequence
  Synthetic SOX2 TCR#10 CDR3beta
ASSRGPEAF

SEQ ID NO: 101 - protein, 11 aa, Artificial Sequence
  Synthetic SOX2 TCR#10 CDR3beta-junction
CASSRGPEAFF

SEQ ID NO: 102 - protein, 113 aa, Artificial Sequence
  Synthetic SOX2 TCR#10 TCRValpha
GEDVEQSLFLSVREGDSSVINCTYTDSSSTYLYWYKQEPGAGLQLLTYIFSNMDMKQDQRLTVLLNKKDKHLSLRIADTQTGDSAIYFCAGPLGGSQGNLIFGKGTKLSVKPN

SEQ ID NO: 103 - protein, 6 aa, Artificial Sequence
  Synthetic SOX2 TCR#10 CDR1alpha
DSSSTY

SEQ ID NO: 104 - protein, 7 aa, Artificial Sequence
  Synthetic SOX2 TCR#10 CDR2alpha
IFSNMDM

SEQ ID NO: 105 - protein, 12 aa, Artificial Sequence
  Synthetic SOX2 TCR#10 CDR3alpha
AGPLGGSQGNLI

SEQ ID NO: 106 - protein, 14 aa, Artificial Sequence
  Synthetic SOX2 TCR#10 CDR3alpha-junction
CAGPLGGSQGNLIF

SEQ ID NO: 107 - DNA, 1839 bp, Artificial Sequence
  Synthetic SOX2 TCR#11 TCRbeta-P2A-TCRalpha, codon-optimized nucleotide
atgggacctcagcttcttggatacgttgtgctgtgtctgcttggagccggacctttagaggctcaggttacccagaaccccagatacctgattacagtgacaggcaagaagctgaccgtgacctgtagccagaacatgaaccacgagtacatgagctggtacagacaggatcctggactgggactccggcaaatttactacagcatgaacgtggaggtgaccgataaaggagatgtgcccgaaggctacaaggtgagcaggaaggagaagaggaatttccccctgatcctggaatctcctagccctaatcagaccagcctgtacttctgtgccagcagcccaaggtctcccgaaggaggaagagcctacaatgagcagttctttggccctggaacaagactgaccgtgctggaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggagaagatgctggagtgtgcgttcatcgttctgtggctgcaacttggatggctgtctggagaggatcaggttacacagtctcctgaagccctgagactgcaagaaggagagagctcatcgttgaactgcagctacacagtgtctggactgagaggcctgttctggtataggcaggatccgggcaaaggacctgagttcctgtttaccctctattcagctggggaggagaaggagaaggagagactgaaggctacactgaccaagaaggagagctttcttcacattactgcccccaaacctgaggattctgccacatacctgtgtgctgctctggacagagacaattatggccaaaatttcgtgtttggccctggaaccagactgtctgtgctgccttacattcagaaccccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 108 - protein, 612 aa, Artificial Sequence
  Synthetic SOX2 TCR#11 TCRbeta-P2A-TCRalpha
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPRSPEGGRAYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAALDRDNYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 109 - protein, 119 aa, Artificial Sequence
  Synthetic SOX2 TCR#11 TCRVbeta
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPRSPEGGRAYNEQFFGPGTRLTVLE

SEQ ID NO: 110 - protein, 5 aa, Artificial Sequence
  Synthetic SOX2 TCR#11 CDR1beta
MNHEY

SEQ ID NO: 111 - protein, 6 aa, Artificial Sequence
  Synthetic SOX2 TCR#11 CDR2beta
SMNVEV

SEQ ID NO: 112 - protein, 17 aa, Artificial Sequence
  Synthetic SOX2 TCR#11 CDR3beta
ASSPRSPEGGRAYNEQF

SEQ ID NO: 113 - protein, 19 aa, Artificial Sequence
  Synthetic SOX2 TCR#11 CDR3beta-junction
CASSPRSPEGGRAYNEQFF

SEQ ID NO: 114 - protein, 113 aa, Artificial Sequence
  Synthetic SOX2 TCR#11 TCRValpha
EDQVTQSPEALRLQEGESSSLNCSYTVSGLRGLFWYRQDPGKGPEFLFTLYSAGEEKEKERLKATLTKKESFLHITAPKPEDSATYLCAALDRDNYGQNFVFGPGTRLSVLPY

SEQ ID NO: 115 - protein, 6 aa, Artificial Sequence
  Synthetic SOX2 TCR#11 CDR1alpha
VSGLRG

SEQ ID NO: 116 - protein, 7 aa, Artificial Sequence
  Synthetic SOX2 TCR#11 CDR2alpha
LYSAGEE

SEQ ID NO: 117 - protein, 13 aa, Artificial Sequence
  Synthetic SOX2 TCR#11 CDR3alpha
AALDRDNYGQNFV

SEQ ID NO: 118 - protein, 15 aa, Artificial Sequence
  Synthetic SOX2 TCR#11 CDR3alpha-junction
CAALDRDNYGQNFVF

SEQ ID NO: 119 - DNA, 1797 bp, Artificial Sequence
  Synthetic SOX2 TCR#13 TCRbeta-P2A-TCRalpha, codon-optimized nucleotide
atgaccattcgactgctgtgctacatgggcttctacttcctgggagctggactgatggaagccgacatctaccaaacacccagatacctggtgatcggaacaggcaagaagatcaccctggagtgtagccagacaatgggacacgacaagatgtactggtaccagcaggatcctggaatggaactgcacctgatccactacagctatggcgtgaacagcacagagaaaggcgatctgagcagcgagtctacagtgagcagaatcagaaccgagcacttccctctgacactggaatctgctagacctagccacacaagccagtacctgtgtgcttctagcgagaacagcaatcagccccagcatttcggcgatggcacccgcttgtccatcttggaggatctgaacaaggtgttccccccagaggtggccgtgttcgagccttctgaggccgagatctcccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccacaatcctgtacgagatcctgctgggcaaggccaccctgtacgccgtgctggtgtctgccctggtgctgatggccatggtcaagcggaaggacttcggttccggagccacgaacttctctctgttaaagcaagcaggagacgtggaagaaaaccccggtcccatgaccagcattagagccgtgttcatcttcctgtggctgcagctggatctggtgaatggcgagaatgtggaacagcatcctagcacactgtctgttcaggagggcgattctgccgtgatcaagtgcacctatagcgattctgccagcaactacttcccttggtacaagcaggagctgggcaaaggacctcagctgatcatcgacatcaggagcaatgtgggagagaagaaggatcagagaatcgccgtgacattaaataagaccgccaagcactttagcctgcacatcacagagacacaacctgaggattctgccgtgtacttttgtgccgcccctaacgacatgagattcggcgccgggacacgtctgacagtgaagcccaacatccaaaatcctgatcccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 120 - protein, 598 aa, Artificial Sequence
  Synthetic SOX2 TCR#13 TCRbeta-P2A-TCRalpha
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSENSNQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAAPNDMRFGAGTRLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 121 - protein, 113 aa, Artificial Sequence
  Synthetic SOX2 TCR#13 TCRVbeta
EADIYQTPRYLVIGTGKKITLECSQTMGHDKMYWYQQDPGMELHLIHYSYGVNSTEKGDLSSESTVSRIRTEHFPLTLESARPSHTSQYLCASSENSNQPQHFGDGTRLSILE

SEQ ID NO: 122 - protein, 5 aa, Artificial Sequence
  Synthetic SOX2 TCR#13 CDR1beta
MGHDK

SEQ ID NO: 123 - protein, 6 aa, Artificial Sequence
  Synthetic SOX2 TCR#13 CDR2beta
SYGVNS

SEQ ID NO: 124 - protein, 11 aa, Artificial Sequence
  Synthetic SOX2 TCR#13 CDR3beta
ASSENSNQPQH

SEQ ID NO: 125 - protein, 13 aa, Artificial Sequence
  Synthetic SOX2 TCR#13 CDR3beta-junction
CASSENSNQPQHF

SEQ ID NO: 126 - protein, 109 aa, Artificial Sequence
  Synthetic SOX2 TCR#13 TCRValpha
GENVEQHPSTLSVQEGDSAVIKCTYSDSASNYFPWYKQELGKGPQLIIDIRSNVGEKKDQRIAVTLNKTAKHFSLHITETQPEDSAVYFCAAPNDMRFGAGTRLTVKPN

SEQ ID NO: 127 - protein, 6 aa, Artificial Sequence
  Synthetic SOX2 TCR#13 CDR1alpha
DSASNY

SEQ ID NO: 128 - protein, 7 aa, Artificial Sequence
  Synthetic SOX2 TCR#13 CDR2alpha
IRSNVGE

SEQ ID NO: 129 - protein, 7 aa, Artificial Sequence
  Synthetic SOX2 TCR#13 CDR3alpha
AAPNDMR

SEQ ID NO: 130 - protein, 9 aa, Artificial Sequence
  Synthetic SOX2 TCR#13 CDR3alpha-junction
CAAPNDMRF

SEQ ID NO: 131 - DNA, 1824 bp, Artificial Sequence
  Synthetic SOX2 TCR#15 TCRbeta-P2A-TCRalpha, codon-optimized nucleotide
atgggcacatctcttctctgttgggttgtgctgggctttctgggcacagatcatacaggagctggagttagccagtctcctaggtataaggtgaccaagaggggacaggatgtggctctgagatgtgaccctatttctggccacgtgagcctttactggtacagacaagctctgggacaaggacccgagtttctgacctacttcaactacgaggcccagcaggataaatctggactgcccaacgacagattcagcgccgaaagacctgaaggctctatcagcacactgaccattcagagaacagagcagagggattctgccatgtacagatgcgccagctctctttggggaagagccgttgatgagcagttttttggccctggaaccagactgacagttctggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatgaactactctcctggccttgtcagcctgattctgctgctgctgggaagaacaagaggcgatagcgttacacagatggaaggacctgtgacactgtctgaagaggccttcctgaccatcaattgcacctacacagccactggttacccgtcccttttctggtatgtgcagtaccctggagagggactgcaactgctgttaaaggccacaaaagccgacgataagggcagcaataagggctttgaggccacctacaggaaggagacaaccagctttcatctggagaagggaagcgtgcaagtgtctgattcagccgtgtacttttgtgccctgctgggctctaatgactacaagctgagctttggagccggcacaacagtgaccgttagagccaatatccaaaaccccgacccagctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 132 - protein, 607 aa, Artificial Sequence
  Synthetic SOX2 TCR#15 TCRbeta-P2A-TCRalpha
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLWGRAVDEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALLGSNDYKLSFGAGTTVTVRANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 133 - protein, 116 aa, Artificial Sequence
  Synthetic SOX2 TCR#15 TCRVbeta
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLWGRAVDEQFFGPGTRLTVLE

SEQ ID NO: 134 - protein, 5 aa, Artificial Sequence
  Synthetic SOX2 TCR#15 CDR1beta
SGHVS

SEQ ID NO: 135 - protein, 6 aa, Artificial Sequence
  Synthetic SOX2 TCR#15 CDR2beta
FNYEAQ

SEQ ID NO: 136 - protein, 13 aa, Artificial Sequence
  Synthetic SOX2 TCR#15 CDR3beta
ASSLWGRAVDEQF

SEQ ID NO: 137 - protein, 15 aa, Artificial Sequence
  Synthetic SOX2 TCR#15 CR3beta-junction
CASSLWGRAVDEQFF

SEQ ID NO: 138 - protein, 113 aa, Artificial Sequence
  Synthetic SOX2 TCR#15 TCRValpha
GDSVTQMEGPVTLSEEAFLTINCTYTATGYPSLFWYVQYPGEGLQLLLKATKADDKGSNKGFEATYRKETTSFHLEKGSVQVSDSAVYFCALLGSNDYKLSFGAGTTVTVRAN

SEQ ID NO: 139 - protein, 6 aa, Artificial Sequence
  Synthetic SOX2 TCR#15 CDR1alpha
ATGYPS

SEQ ID NO: 140 - protein, 7 aa, Artificial Sequence
  Synthetic SOX2 TCR#15 CDR2alpha
ATKADDK

SEQ ID NO: 141 - protein, 11 aa, Artificial Sequence
  Synthetic SOX2 TCR#15 CDR3alpha
ALLGSNDYKLS

SEQ ID NO: 142 - protein, 13 aa, Artificial Sequence
  Synthetic SOX2 TCR#15 CDR3alpha-junction
CALLGSNDYKLSF

SEQ ID NO: 143 - DNA, 1815 bp, Artificial Sequence
  Synthetic SOX2 TCR#16 TCRbeta-P2A-TCRalpha, codon-optimized nucleotide
atgtctatcggtctgctgtgctgtgtagcattcagtctactttgggcttctcccgtgaatgctggagttacacaaacccccaagttccaggtgttaaagaccggccagagcatgacactgcagtgtgctcaggacatgaatcacaacagtatgtattggtacagacaggatcccggcatgggactgagactgatctactacagcgcttctgagggaactacagacaaaggagaggtgcccaacggctacaacgtgagcaggctgaacaagagggagtttagcctgagactggaatccgctgccccgtctcagacaagcgtgtacttctgcgcctcttctgagggatacggctatgagcagtattttggccctggcacaagactgacagtgacagaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatgctgctggaacacctactgatcatcctgtggatgcagctgacatgggtgtctggacagcagctgaatcagtctcctcagagcatgttcattcaggagggagaggatgtgagcatgaactgcacatcgtccagcatcttcaacacctggctgtggtataagcaggatcctggagaaggacctgtgctgctgattgccctgtataaagccggagagctgacctctaatggaagactgacagcccagtttggcatcaccagaaaggacagcttcctgaacatctccgcatccattccttctgatgtgggcatctacttctgtgccggccacattacacaaggcggatctgagaagctggtgtttggcaaaggcaccaagctgacagttaacccctacatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 144 - protein, 604 aa, Artificial Sequence
  Synthetic SOX2 TCR#16 TCRbeta-P2A-TCRalpha
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEGYGYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHITQGGSEKLVFGKGTKLTVNPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 145 - protein, 113 aa, Artificial Sequence
  Synthetic SOX2 TCR#16 TCRVbeta
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEGYGYEQYFGPGTRLTVTE

SEQ ID NO: 146 - protein, 5 aa, Artificial Sequence
  Synthetic SOX2 TCR#16 CDR1beta
MNHNS

SEQ ID NO: 147 - protein, 6 aa, Artificial Sequence
  Synthetic SOX2 TCR#16 CDR2beta
SASEGT

SEQ ID NO: 148 - protein, 11 aa, Artificial Sequence
  Synthetic SOX2 TCR#16 CDR3beta
ASSEGYGYEQY

SEQ ID NO: 149 - protein, 13 aa, Artificial Sequence
  Synthetic SOX2 TCR#16 CDR3beta-junction
CASSEGYGYEQYF

SEQ ID NO: 150 - protein, 114 aa, Artificial Sequence
  Synthetic SOX2 TCR#16 TCRValpha
GQQLNQSPQSMFIQEGEDVSMNCTSSSIFNTWLWYKQDPGEGPVLLIALYKAGELTSNGRLTAQFGITRKDSFLNISASIPSDVGIYFCAGHITQGGSEKLVFGKGTKLTVNPY

SEQ ID NO: 151 - protein, 5 aa, Artificial Sequence
  Synthetic SOX2 TCR#16 CDR1alpha
SIFNT

SEQ ID NO: 152 - protein, 7 aa, Artificial Sequence
  Synthetic SOX2 TCR#16 CDR2alpha
LYKAGEL

SEQ ID NO: 153 - protein, 13 aa, Artificial Sequence
  Synthetic SOX2 TCR#16 CDR3alpha
AGHITQGGSEKLV

SEQ ID NO: 154 - protein, 15 aa, Artificial Sequence
  Synthetic SOX2 TCR#16 CDR3alpha-junction
CAGHITQGGSEKLVF

SEQ ID NO: 155 - protein, 140 aa, Artificial sequence
  Synthetic TCR Calpha1
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 156 - protein, 178 aa, Artificial sequence
  Synthetic TCR Cbeta2
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 157 - protein, 176 aa, Artificial sequence
  Synthetic TCR beta1
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 158 - protein, 214 aa, Artificial sequence
  Synthetic CD8 co-receptor alpha chain, without signal sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 159 - protein, 189 aa, Artificial sequence
  Synthetic CD8 co-receptor beta chain isoform 1, without signal sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 160 - protein, 200 aa, Artificial sequence
  Synthetic CD8 co-receptor beta chain isoform 2, without signal sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 161 - protein, 225 aa, Artificial sequence
  Synthetic CD8 co-receptor beta chain isoform 3, without signal sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 162 - protein, 225 aa, Artificial sequence; identical to SEQ ID NO: 161
  Synthetic CD8 co-receptor beta chain isoform 4, without signal sequence

SEQ ID NO: 163 - protein, 222 aa, Artificial sequence
  Synthetic CD8 co-receptor beta chain isoform5, without signal sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

(19) United States
(12) Patent Application Publication
(10) Pub. No.: US 2025/0018036 A1
(43) Pub. Date: Jan. 16, 2025

(54) IMMUNOTHERAPY TARGETING SOX2 ANTIGENS

(71) Applicant: Fred Hutchinson Cancer Center, Seattle, WA (US)

(72) Inventors: Tijana Martinov, Seattle, WA (US); Rachel Perret, Wellington (NZ); Philip D. Greenberg, Mercer Island, WA (US); Thomas M. Schmitt, Seattle, WA (US)

(21) Appl. No.: 18/246,502

(22) PCT Filed: Sep. 23, 2021
(86) PCT No.: PCT/US2021/051832

§ 371 Date: Mar. 23, 2023

Related U.S. Application Data
(60) Provisional application No. 63/083,069, filed on Sep. 24, 2020.

(51) Int. Cl. A61K 39/00; A61P 35/00; C07K 14/705; C07K 14/725; C07K 16/28; C12N 5/0783

(52) U.S. Cl. CPC A61K 39/464411; A61K 39/4611; A61K 39/4632; A61P 35/00; C07K 14/7051; C07K 14/70517; C07K 16/2833; C12N 5/0636; A61K 2039/5156

(57) ABSTRACT

The present disclosure provides compositions and methods for targeting SOX2 antigen to, for example, treat or manage cancer. Provided compositions include binding proteins that are capable of binding to a SOX2 antigen:HLA complex. Also provided are polynucleotides and transgene constructs encoding binding proteins, such as a T cell receptor or a chimeric antigen receptor, that specifically bind to a SOX2 antigen. Such polynucleotides and transgene constructs can be introduced into an immune cell, such as a T cell, and used in immunotherapy in a subject having or at risk for a cancer associated with SOX2 expression or activity. The present disclosure also provides immunogenic compositions comprising SOX2 antigens, and related uses.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_47701USPC_SEQUENCE_LISTING.txt. The text file is 158 KB, was created on Mar. 22, 2023, and is being submitted electronically.

### BACKGROUND

Various malignancies including multiple myeloma have poor clinical outcomes, and recurrent disease following standard-of-care therapy is associated with extremely poor prognosis. Standard treatment regimens have involved surgery, radiation therapy, and/or chemotherapy; however, these approaches are not curative for all patients and can be accompanied by toxicities. Accordingly, new strategies are needed for targeting difficult-to-treat cancers. The present disclosure addresses such needs, and further provides other related advantages.

### BRIEF DESCRIPTION OF THE FIGURES

FIG. 1 provides flow cytometry plots showing the percentage of IFN-γ-producing T cells in (top) an exemplary CD8⁺ T cell line that was SOX2-reactive, and (bottom) an exemplary CD8⁺ T cell line that was not SOX2-reactive, following stimulation with 1 μg/ml SOX2 peptides, media, or Cell Stimulation Cocktail for 4 hours in the presence of Golgi plug and Golgi block. Data are from Days 10-14, following 3 rounds of stimulation.

FIG. 2 provides flow cytometry plots showing percentage of IFN-γ-producing T cells from an exemplary CD8⁺ T cell line that was SOX2-reactive and HLA-A*02:01-restricted. Data are from Days 10-14, following three rounds of stimulation with with 1 μg/ml SOX2 peptides, media, or T2 cells pre-loaded with SOX2 peptides or media, for 4 hours in the presence of Golgi plug and Golgi block.

FIG. 3 provides flow cytometry plots showing percentage of IFN-γ-producing T cells in CD8⁺ T cell lines that responded to six different SOX2 peptides (as indicated).

FIG. 4 provides data showing that 293E target cells modified to express either the standard proteasome (293E-SP) or the immunoproteasome (293E-IP) express SOX2.

FIG. 5 provides a diagram showing an experimental setup for a killing assay (IncuCyte) using labeled SOX2⁺ 293E target cells, as described in the Examples.

FIG. 6 provides data from an IncuCyte killing assay showing that SOX2 (277-287) and SOX2 (58-66) epitopes are processed by the standard proteasome (SP). Wells were imaged over a two day period in an IncuCyte S3 system, and the amount of RapidRed⁺ Caspase 3/7 green⁺ target cells was quantified.

FIG. 7 provides data from an IncuCyte killing assay showing that SOX2 (277-287) and SOX2 (58-66) epitopes are processed by the immunoproteasome (IP). Wells were imaged over a two day period in an IncuCyte S3 system, and the amount of RapidRed⁺ Caspase 3/7 green⁺ target cells was quantified.

FIG. 8 provides data from an IncuCyte killing assay showing that SOX2 (277-287)-specific T cells kill SOX2⁺ plasma cell leukemia cells. Wells were imaged over a two day period in an IncuCyte S3 system, and the amount of RapidRed⁺ Caspase 3/7 green⁺ target cells was quantified.

FIG. 9 provides data from an IncuCyte killing assay showing that SOX2 (277-287)-specific T cells kill SOX2⁺ ovarian cancer cells. Wells were imaged over a two day period in an IncuCyte S3 system, and the amount of RapidRed⁺ Caspase 3/7 green⁺ target cells was quantified.

FIG. 10 shows data from a peptide:HLA tetramer gating assay used to sort for SOX2-specific CD8⁺ T cells.

FIGS. 11A-11M provide additional data and experimental designs related to exemplary SOX2-specific T cells according to the present disclosure, and uses of the same. FIG. 11A shows (top) an experimental scheme for assessing IFN-γ production by CD8+ T cells line in response to a pool of SOX2 peptides, and (bottom) representative data. FIG. 11B shows an experimental scheme for assessing IFN-γ production by CD8+ T cell lines in response to SOX2 peptides in a concentration range. FIGS. 11C and 11D show data from certain T cell lines using the experimental scheme shown in FIG. 11B. FIG. 11E shows an experimental scheme for assessing IFN-γ production by CD8+ T cell lines in response to T2 cells pulsed with SOX2 peptide(s). FIG. 11F shows data from certain T cell lines using the experimental scheme shown in FIG. 11E. FIG. 11G shows an experimental scheme to assess CD137 expression (as a measure of activation) in CD8+ T cells expanded in the presence of SOX2 peptides. FIG. 11H shows data from a T cell line using the experimental scheme shown in FIG. 11G. FIG. 11I shows an experimental scheme to assess killing activity by CD8+ T cell lines expanded in the presence of SOX2 peptides, using labeled L363 target cells in an IncuCyte® killing assay. FIG. 11J shows data from certain T cell lines using the experimental scheme shown in FIG. 11I. FIG. 11K shows an experimental scheme for injecting L363 cells into a humanized mouse model. FIG. 11L shows survival over time of humanized mice receiving one or another dose of the L363 cells, or no cells. FIG. 11M shows stable SOX2 expression in L363 cells in vivo (3 mice) or in vitro.

FIG. 12 shows functional avidity (peptide antigen logEC50) of certain SOX2 277-287-specific TCRs. “TCR2” comprises the Vα amino acid sequence of SEQ ID NO.:30 and the Vβ amino acid sequence of SEQ ID NO.:25. “TCR4” comprises the Vα amino acid sequence of SEQ ID NO.42 and the Vβ amino acid sequence of SEQ ID NO.:37. “TCR5” comprises the Vα amino acid sequence of SEQ ID NO.:54 and the Vβ amino acid sequence of SEQ ID NO.49. “TCR6” comprises the Vα amino acid sequence of SEQ ID NO.:66 and the Vβ amino acid sequence of SEQ ID NO.:61. “TCR9” comprises the Vα amino acid sequence of SEQ ID NO.:90 and the Vβ amino acid sequence of SEQ ID NO.:85. “TCR10” comprises the Vα amino acid sequence of SEQ ID NO.:102 and the Vβ amino acid sequence of SEQ ID NO.:97. “TCR11” comprises the Vα amino acid sequence of SEQ ID NO.:114 and the Vβ amino acid sequence of SEQ ID NO.:109. “TCR13” comprises the Vα amino acid sequence of SEQ ID NO.:126 and the Vβ amino acid sequence of SEQ ID NO.:121.

FIG. 13 shows a diagram showing a setup of an overnight T cell:tumor cell co-culture testing recognition by T cells of endogenously processed and presented SOX2 epitope.

FIGS. 14 and 14A-14D show recognition (percent of T cells expressing CD137) of T cells transduced with TCR5 or TCR10 in response to SOX2+ tumor cell lines. FIGS. 14A-14D show enhanced views of portions of FIG. 14.

### DETAILED DESCRIPTION

The present disclosure generally relates to SOX2 antigens and binding proteins and immune cells (e.g., T cells) specific for SOX2 antigens. By way of background, immunotherapies can be effective against some cancers. For example, T cell immunotherapy can be highly effective against hematologic malignancies, as exemplified by the impressive results of clinical trials evaluating therapeutic T cells genetically modified with chimeric antigen receptors (CARs) targeting CD19 for acute lymphoblastic leukemia (ALL). However, some CARs that comprise a binding domain from an antibody (e.g., scFv, Fab) are limited to targeting cell surface-expressed molecules, and identifying target cell surface antigens with disease-specific expression patterns (i.e., that are not also expressed on healthy cells and tissues) can be a challenge. Alternative immunotherapy strategies include administration or eliciting of of T cells naturally expressing, or modified to express, T cell receptors (TCRs, including engineered TCRs) specific for peptide antigens derived from endogenous cell proteins and presented on the cell surface in association with HLA molecules.

Selecting an appropriate antigen target is important for effective immunotherapy. Cell surface antigens (e.g., CD19, BCMA, or CD138 in the context of multiple myeloma) frequently decrease in expression following a treatment, potentially permitting outgrowth of antigen-low or antigen-negative cells and relapse of disease. Accordingly, successful antigen targets for immunotherapy include those that are selectively expressed on tumors, presented by widely expressed HLA alleles that are highly prevalent in the disease population, and involved in the induction or maintenance of a malignant phenotype.

The present disclosure teaches that SOX2 is one such candidate antigen target for cancer immunotherapy. The present disclosure provides, in part, antigenic peptides from SOX2 that are capable of eliciting an immune cell response and are expressed in certain cancers, including, for example, multiple myeloma, plasma cell leukemia, ovarian cancer, glioma, lung cancer, neck cancer, and cervical cancer.

In certain embodiments, the antigenic peptides are, unexpectedly and advantageously, processed via the standard proteasome (SP) processing pathway, which is used by cancer cells in certain nonhematopoietic malignancies, as well as processed via the immunoproteasome (IP) processing pathway, which is used by cancer cells in certain hematological cancers, but is in some cases less used or is not used by solid tumor cells.

Accordingly, in some embodiments, the antigenic peptides are useful for targeting a range of malignancies, whether malignant cells utilize SP processing, IP processing, or both.

The present disclosure also provides binding proteins that are capable of binding to a SOX2 antigen (e.g., in the context of a peptide:HLA compex). The binding proteins are capable (e.g., when expressed by a host cell such as an immune cell (e.g. a T cell)) of binding and promoting the killing of cells presenting such antigens (e.g., in complex with an HLA molecule, such as HLA-A*02:01). In some embodiments, binding comprises specific binding as disclosed herein. The present disclosure further provides host cells (e.g., immune cells such as T cells) that encode and are capable of expressing SOX2-specific binding proteins that are capable of binding to a peptide containing a SOX2 antigen, and to isolated polynucleotides encoding a binding protein. In certain embodiments, there are provided modified immune cells that comprise polynucleotides encoding binding proteins that are capable of binding to a peptide containing a SOX2 antigen of this disclosure, and uses of the presently disclosed compositions for treating disease (e.g., cancer), eliciting immune responses, or identifying antigen-reactive cells, among other uses.

The presently disclosed binding proteins, polynucleotides, vectors, host cells, compositions, and methods are useful for treating a solid tumor, a hematological malignancy, or both. In certain embodiments, the presently disclosed binding proteins, polynucleotides, vectors, host cells, compositions, and methods are useful for treating a cancer in which cancer cells express SOX2 and express the standard proteasome, or in which cancer cells express SOX2 and the immunoproteasome, or in which cancer cells express SOX2 and the standard proteasome and the immunoproteasome.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include”, “have”, and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

An “immunoglobulin superfamily binding protein” or “IgSF binding protein” refers to a cell surface or soluble protein that is involved in recognition of, binding to, and/or adhering to a target (e.g., cell, antigen, soluble factor) and comprises one or more immunologlobulin domain and/or immunoglobulin fold. IgSF binding proteins of the present disclosure comprise an antigen-recognition domain, such as a variable domain or variable region, such as is found in an antibody or antigen-binding fragment thereof (of any isotype), or in a T cell receptor or antigen-binding fragment thereof (e.g., an IgV region). IgSF proteins can possess a native binding specificity for a target, or can be engineered to have or enhance binding specificity and/or affinity for the target. Other IgSF proteins for use in the present disclosure include, for example, proteins comprising an IgC1 domain, an IgC2 domain, and/or an IgI domain; a killer-cell immunoglobulin-like receptor (KIR); a leukocyte immunoglobulin-like receptor (LILR); a cell adhesion molecule (CAM); and combinations of these.

As used herein, the terms “immune system cell” or “immune cell” mean any cell of the immune system that originates from a hematopoietic stem cell in the bone marrow, which gives rise to two major lineages, a myeloid progenitor cell (which give rise to myeloid cells such as monocytes, macrophages, dendritic cells, meagakaryocytes and granulocytes) and a lymphoid progenitor cell (which give rise to lymphoid cells such as T cells, B cells and natural killer (NK) cells). Exemplary immune system cells include a CD4⁺ T cell, a CD8⁺ T cell, a CD4−CD8− double negative T cell, a γδ T cell, a regulatory T cell, a stem cell memory T cell, a natural killer cell, a natural killer T cell, and a dendritic cell. Macrophages and dendritic cells can be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs). T cells can be naïve (“TN”; not exposed to antigen; increased expression of CD62L, CCR7, CD28, CD3, CD127, and CD45RA, and decreased or no expression of CD45RO as compared to TCM (described herein)), memory T Cells™ (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8⁺ cytotoxic T lymphocytes that express CD45RA, have decreased expression of CD62L, CCR7, and CD28 as compared to TCM, and are positive for granzyme and perforin. Helper T cells (TH) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺CD25⁺(Foxp3⁺) regulatory T cells and Treg17 cells, as well as Tr1, Th3, CD8⁺CD28⁻, and Qa-1 restricted T cells.

The term “T cell receptor” (TCR) refers to an immunoglobulin superfamily member (having a variable binding domain, a constant domain, a transmembrane region, and a short cytoplasmic tail; see, e. g., Janeway et al., Immunobiology: The Immune System in Health and Disease, 3rd Ed., Current Biology Publications, p. 433, 1997) capable of specifically binding to an antigen peptide bound to a MHC receptor. A TCR can be found on the surface of a cell or in soluble form and generally is comprised of a heterodimer having α and β polypeptides (also known as TCR α and TCRβ, respectively), or γ and δ polypeptides (also known as TCRγ and TCRδ, respectively).

Like other immunoglobulins (e.g., antibodies), the extracellular portion of TCR polypeptides (e.g., α-polypeptides, β-polypeptides) contain two immunoglobulin domains, a variable domain (e.g., α-polypeptide variable domain or V_{α} polypeptide or V_{β} polypeptide; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically 5 amino acids 117 to 259 based on Kabat, β-chain constant domain or C_{β}, typically amino acids 117 to 295 based on Kabat) adjacent the cell membrane. Also, like antibodies, the variable domains contain complementary determining regions (CDRs) separated by framework regions (FRs) (see, e.g., Jores et al., Proc. Nat'l Acad. Sci. USA 87:9138, 1990; Chothia et al., EMBO J. 7:3745, 1988; see also Lefranc et al., Dev. Comp. Immunol. 27:55, 2003). The source of a TCR as used in the present disclosure may be from any of a variety of animal species, such as a human, mouse, rat, rabbit, or other mammal.

The term “variable region” or “variable domain” refers to the domain of an immunoglobulin superfamily binding protein (e.g., a TCR α-polypeptide or β-polypeptide (or γ polypeptide and δ polypeptide for γδ TCRs)) that is involved in binding of the immunoglobulin superfamily binding protein (e.g., TCR, antibody) to antigen. The variable domains of the α-polypeptide and β-polypeptide (Vα and Vβ, respectively) of a native TCR generally have similar structures, with each domain comprising four conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

The terms “complementarity determining region,” and “CDR,” are synonymous with “hypervariable region” or “HVR,” and are known in the art to refer to sequences of amino acids within immunoglobulin (e.g., TCR) variable regions, which confer antigen specificity and/or binding affinity and are separated from one another by framework regions. In general, there are three CDRs in each TCR α-polypeptide variable region (αCDR1, αCDR2, αCDR3) and three CDRs in each TCR β-polypeptide variable region (βCDR1, βCDR2, βCDR3). In the case of antibodies (except for heavy-chain-only antibodies), both heavy chain and light chain variable regions are present, and each of these comprises three CDRs (CDRH1, CDRH2, CDRH3, CDRL1, CDRL2, CDRL3).

In TCRs, CDR3 is thought to be the main CDR responsible for recognizing processed antigen. Typically, CDR1 and CDR2 mainly interact with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced. Compared with CDR1 and CDR2, CDR3 is typically significantly more diverse because of the addition and loss of nucleotides during the recombination process. This is typically the case for CDR3β in TCRs and CDRH3 in antibodies.

TCR and antibody variable domain sequences can be aligned to a numbering scheme (e.g., Kabat, Chothia, Enhanced Chothia, EU, IMGT, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, variable domain sequences are according to the IMGT numbering scheme (see Lefranc et al., Dev. Comp. Immunol. 27:55, 2003 and imgt.org). In some embodiments, a CDR sequence (amino acid or encoding nucleotide) of a TCR includes sequence (amino acid or encoding nucleotide) encoded by or at the junction of V and J alleles or at the junction of V and D alleles or at the junction of D and J alleles. IMGT junctions are recognized by those having ordinary skill in the art. For example, in the TCR Vβ sequence set forth in SEQ ID NO.:25:

DVKVTQSSRYLVKRTGEKVFLECVQDMDHENMFWYRQDPGLGLRLIYFSY
DVKMKEKGDIPEGYSVSREKKERFSLILESASTNQTSMYLCASSLILAGR
NTGELFFGEGSRLTVLE,

the IMGT CDR3 amino acid sequence is ASSLILAGRNTGELF (SEQ ID NO.:28) and the IMGT CDR3 amino acid sequence inclusive of the junction amino acids is CASSLILAGRNTGELFF SEQ ID NO.:29. Certain CDR sequences disclosed herein comprise the junction amino acids.

In certain embodiments, a TCR is found on the surface of a T cell (or a T lymphocyte) and associates with a CD3 complex. “CD3” is a multi-protein complex of six polypeptides (see, Abbas and Lichtman, 2003; Janeway et al., p. 172 and 178, 1999) that is associated with antigen signaling in T cells. In mammals, the complex comprises a CD3γ chain, a CD3δ polypeptide, two CD3ε polypeptides, and a homodimer of CD3ζ polypeptides. The CD3γ, CD3β, and CD3ε polypeptides are related cell surface proteins of the immunoglobulin superfamily containing a single immunoglobulin domain. The transmembrane regions of the CD3γ, CD3β, and CD3ε polypeptides are negatively charged, which is believed to allow these polypeptides to associate with the positively charged T cell receptor polypeptides. The intracellular tails of the CD3γ, CD3β, and CD3ε polypeptides each contain a single conserved motif known as an immunoreceptor tyrosine based activation motif or ITAM, whereas each CD3ζ chain has three ITAMs. Without wishing to be bound by theory, it is believed that ITAMs are important for the signaling capacity of a TCR complex. CD3 as used in the present disclosure may be from various animal species, including human, mouse, rat, or other mammals.

As used herein, the term “TCR complex” refers to a complex formed by the association of CD3 with TCR. For example, a TCR complex can be composed of a CD3γ polypeptide, a CD3β polypeptide, two CD3ε polypeptides, a homodimer of CD3ζ polypeptides, a TCRα polypeptide, and a TCRβ polypeptide. Alternatively, a TCR complex can be composed of a CD3γ chain, a CD3β chain, two CD3ε chains, a homodimer of CD3ζ chains, a TCRγ chain, and a TCRβ chain.

A “component of a TCR complex”, as used herein, refers to a TCβ chain (i.e., TCRα, TCRβ, TCRγ or TCRδ), a CD3 chain (i.e., CD3γ, CD3δ, CD3ε or CD3ζ), or a complex formed by two or more TCR polypeptides or CD3 polypeptides (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε polypeptides).

“Chimeric antigen receptor” (CAR) refers to a fusion protein that is engineered to contain two or more naturally occurring amino acid sequences linked together in a way that does not occur naturally or does not occur naturally in a host cell, which fusion protein can function as a receptor when present on a surface of a cell. CARs can include an extracellular portion comprising an antigen-binding domain (e.g., obtained or derived from an immunoglobulin or immunoglobulin-like molecule, such as a TCR binding domain derived or obtained from a TCR specific for a cancer antigen, an scFv derived or obtained from an antibody, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016), Stone et al., Cancer Immunol. Immunother., 63(11):1163 (2014), and Walseng et al., Scientific Reports 7:10713 (2017), which CAR constructs and methods of making the same are incorporated by reference herein). In some embodiments, CARs of the present disclosure that specifically bind to an antigen (e.g., in the context of a peptide:HLA complex) comprise a TCR Vα domain and a Vβ domain.

As used herein, “fusion protein” or “fusion polypeptide” refers to a protein that, in a single chain, has at least two distinct domains, sequences, motifs, wherein the domains, sequences, or motifs are not naturally found together (e.g., in the specified arrangement, order, or number, or at all) in a protein. In certain embodiments, a fusion protein comprises at least two distinct domains or motifs that are not naturally found together in a single peptide or polypeptide. A polynucleotide encoding a fusion protein may be constructed using PCR, recombinantly engineered, or the like, or such fusion proteins can be synthesized.

“Antigen” or “Ag” as used herein refers to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen, or that endogenously (e.g., without modification or genetic engineering by human intervention) express a mutation or polymorphism that is immunogenic.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

“SOX2”, also known in the art as “SRY-2” and “sex determining region Y”, is a transcription factor that is involved in self-renewal of undifferentiated embryonic stem cells, in maintenance of embryonic stem cells and neural stem cells, and in the malignant phenotype of certain cancers. SOX2 is a member of the Sox family of transcription factors, which share high-mobility group (HMG) box domains of approximately 80 amino acids. The amino acid sequence of human SOX2 is provided in SEQ ID NO:1.

As used herein, the term “SOX2 antigen” refers to a naturally or synthetically produced peptide portion of a SOX2 protein ranging in length from about 7 amino acids to about 25 amino acids, or more. In some embodiments, a SOX2 antigen comprises a length of about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, about 25 amino acids, about 30 amino acids, about 35 amino acids, or more. In some embodiments, a SOX2 antigen comprises a length of 9, 10, 11, 12, 13, 14, 15, 16, 17, 18, 19, 20, 21, 22, 23, 24, or 25 amino acids. In certain embodiments, “SOX2 antigen” is used interchangeably with “SOX2 peptide” or “SOX2 antigen peptide” or “SOX2 peptide antigen”. In some embodiments, a SOX2 antigen comprises or consists of the amino acid sequence set forth in SEQ ID NO:2, 3, 4, 5, 6, or 7. Also contemplated are antigenic amino acid sequence variants of a reference SOX2 antigen that comprises or consists of the amino acid sequence set forth in SEQ ID NO:2, 3, 4, 5, 6, or 7. Variants will typically be of the same length as the reference SOX2 antigen, but may include one or more amino acid changes and/or differences in a post-translational modification as compared to the reference SOX2 antigen, while retaining antigenicity, HLA-compatibility, and general structure and charge characteristics of the reference SOX2 antigen.

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well established (see, e.g., Murphy, Janeway's Immunobiology (8^{th} Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MHC (HLA) molecules, whereas peptides processed in the vesicular system (e.g., bacterial, viral) will vary in length from about 10 amino acids to about 25 amino acids and associate with class II MHC (HLA) molecules.

“Major histocompatibility complex” (MHC) refers to glycoproteins that deliver peptide antigens to a cell surface of all nucleated cells. MHC class I molecules are heterodimers having a membrane spanning a polypeptide (with three a domains) and a non-covalently associated I2 microglobulin. MHC class II molecules are composed of two transmembrane glycoproteins, a and p, both of which span the membrane. Each polypeptide has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA). HLAs corresponding to “class I” MHC present peptides from inside the cell and include, for example, HLA-A, HLA-B, and HLA-C. Alleles include, for example, HLA A*02:01; HLA-A*03:01; HLA-A*11:01; HLA-B*40:01; HLA-B*44:02; or HLA-B*44:03. HLAs corresponding to “class II” MHC present peptides from outside the cell and include, for example, HLA-DP, HLA-DM, HLA-DOA, HLA-DOB, HLA-DQ, and HLA-DR.

As used herein, the term “CD8 co-receptor” or “CD8” means the cell surface glycoprotein CD8, either as an alpha-alpha homodimer or an alpha-beta heterodimer. The CD8 co-receptor assists in the function of cytotoxic T cells (CD8+) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are five (5) known CD8 beta polypeptide isoforms (see UniProtKB identifier P10966) and a single known CD8 alpha polypeptide (see UniProtKB identifier P01732).

“CD4” is an immunoglobulin co-receptor glycoprotein that assists the TCR in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII 02, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce various types of T helper cells.

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “VL” or “VL” and “VH” or “VH” refer to the variable binding region or domain from an antibody light chain and an antibody heavy chain, respectively. In certain embodiments, a VL is a kappa (κ) class (also “VK” herein). In certain embodiments, a VL is a lambda (λ) class. Like TCR variable domains, the variable domains of antibodies comprise CDRs and framework regions (FRs). There are three CDRs in each antibody variable domain (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

TCR-mimic antibodies are antibodies (of any isotype e.g., IgG (1, 2, 3, 4), IgE, IgD, IgA, IgM) that are capable of binding to a peptide:MHC complex (i.e., recognizing the peptide:MHC complex and binding thereto). In some embodiments, TCR-mimic antibodies possess antigen-specific, major histocompatibility complex-compatibility or -restriction similar to that of T-cell receptors. TCR-mimic antibodies may be prepared by the hybridoma methodology described by Kohler et al., Nature 256:495 (1975), or may be made using recombinant DNA methods in bacterial, eukaryotic animal, or plant cells (see, e.g., U.S. Pat. No. 4,816,567). TCR-mimic antibodies may also be isolated from phage antibody libraries using the techniques described in Clackson et al., Nature, 352:624-628 (1991) and Marks et al., J. Mol. Biol., 222:581-597 (1991), and Noy et al. Expert Rev. Anticancer Ther. 5(3):523-536 (2005); these techniques are incorporated herein in their entireties), for example. TCR-mimic antibodies may also be obtained using methods disclosed in PCT Publication No. WO 2004/076677A2. Antigen-binding fragments of TCR-mimic antibodies (e.g., a CDR, a VH, a VL, a Fab, a Fd, or the like) are also contemplated.

Altered domains or altered proteins or derivatives can include those based on all possible codon choices for the same amino acid and codon choices based on conservative amino acid substitutions. For example, the following six groups each contain amino acids that are conservative substitutions for one another: 1) alanine (ala; A), serine (ser; S), threonine (thr; T); 2) aspartic acid (asp; D), glutamic acid (glu; E); 3) asparagine (asn; N), glutamine (gln; Q); 4) arginine (arg; R), lysine (lys; K); 5) Isoleucine (ile; I), leucine (L), methionine (met; M), valine (val; V); and 6) phenylalanine (phe; F), tyrosine (tyr; Y), tryptophan (trp; W). (See also WO97/09433 at page 10, Lehninger, Biochemistry, 2^{nd} Edition, Worth Publishers, Inc., NY, NY, pp. 71-77, 1975; Lewin Genes IV, Oxford University Press, NY and Cell Press, Cambridge, MA, p. 8, 1990; Creighton, Proteins, W.H. Freeman and Company 1984). In addition, individual substitutions, deletions or additions that alter, add or delete, a single amino acid or a small percentage of amino acids in an encoded sequence are also “conservative substitutions.”

As used herein, “nucleic acid” or “nucleic acid molecule” or “polynucleotide” refers to any of deoxyribonucleic acid (DNA), ribonucleic acid (RNA), oligonucleotides, polynucleotides, fragments thereof generated, for example, by the polymerase chain reaction (PCR) or by in vitro translation, and also to fragments generated by any of ligation, scission, endonuclease action, or exonuclease action. In certain embodiments, the nucleic acids of the present disclosure are produced by PCR.

Nucleic acids can be composed of monomers that are naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

As used herein, the terms “recombinant”, “engineered”, and “modified” refer to a cell, microorganism, nucleic acid molecule, polypeptide, protein, plasmid, or vector that has been modified by introduction of an exogenous nucleic acid molecule, or refers to a cell or microorganism that has been genetically engineered by human intervention—that is, modified by introduction of of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or three codons or amino acids, a deletion of one to about 5 codons or amino acids, or a combination thereof.

A “conservative substitution” is recognized in the art as a substitution of one amino acid for another amino acid that has similar properties. Exemplary conservative substitutions are well known in the art (see, e.g., WO 97/09433 at page 10; Lehninger, Biochemistry, 2^{nd} Edition; Worth Publishers, Inc. NY, NY, pp. 71-77, 1975; Lewin, Genes IV, Oxford University Press, NY and Cell Press, Cambridge, MA, p. 8, 1990).

The term “construct” refers to any polynucleotide that contains a recombinant nucleic acid molecule. A “transgene” or “transgene construct” refers to a construct that contains two or more genes operably linked in an arrangement that is not found in nature. The term “operably-linked” (or “operably linked” herein) refers to the association of two or more nucleic acid molecules on a single nucleic acid fragment so that the function of one is affected by the other. For example, a promoter is operably-linked with a coding sequence when it can affect the expression of that coding sequence (i.e., the coding sequence is under the transcriptional control of the promoter). “Unlinked” means that the associated genetic elements are not closely associated with one another and the function of one does not affect the other. In some embodiments, the genes present in a transgene are operably linked to an expression control sequence (e.g., a promoter).

A construct (e.g., a transgene) can be present in a vector (e.g., a bacterial vector, a viral vector) or can be integrated into a genome. A “vector” is a nucleic acid molecule that is capable of transporting another nucleic acid molecule. Vectors can be, for example, plasmids, cosmids, viruses, a RNA vector or a linear or circular DNA or RNA molecule that can include chromosomal, non-chromosomal, semi-synthetic or synthetic nucleic acid molecules. Exemplary vectors are those capable of autonomous replication (episomal vector) or expression of nucleic acid molecules to which they are linked (expression vectors). Vectors useful in the compostions and methods of this disclosure are described further herein.

The term “expression”, as used herein, refers to the process by which a polypeptide is produced based on the encoding sequence of a nucleic acid molecule, such as a gene. The process can include transcription, post-transcriptional control, post-transcriptional modification, translation, post-translational control, post translational modification, or any combination thereof.

The term “introduced” in the context of inserting a nucleic acid molecule into a cell, means “transfection”, or “transformation”, or “transduction” and includes reference to the incorporation of a nucleic acid molecule into a eukaryotic or prokaryotic cell wherein the nucleic acid molecule can be incorporated into the genome of a cell (e.g., a chromosome, a plasmid, a plastid, or a mitochondrial DNA), converted into an autonomous replicon, or transiently expressed (e.g., transfected mRNA).

As used herein, “heterologous” or “exogenous” nucleic acid molecule, construct or sequence refers to a nucleic acid molecule or portion of a nucleic acid molecule that is not native to a host cell, but can be homologous to a nucleic acid molecule or portion of a nucleic acid molecule from the host cell. The source of the heterologous or exogenous nucleic acid molecule, construct or sequence can be from a different genus or species. In certain embodiments, a heterologous or exogenous nucleic acid molecule is added (i.e., not endogenous or native) to a host cell or host genome by, for example, conjugation, transformation, transfection, transduction, electroporation, or the like, wherein the added molecule can integrate into the host genome or exist as extra-chromosomal genetic material (e.g., as a plasmid or other form of self-replicating vector), and can be present in multiple copies. In addition, “heterologous” refers to a non-native enzyme, protein or other activity encoded by an exogenous nucleic acid molecule introduced into the host cell, even if the host cell encodes a homologous protein or activity. It will be appreciated that in the case of a host cell that comprises a heterologous polynucleotide, the polynucleotide is “heterologous” to progeny of the host cell, whether or not the progeny were themselves manipulated (e.g., transduced) to contain the polynucleotide. Such progeny may be referred—to as “modified” host cells, whether the subject host cell was itself modified to comprise the polynucleotide, or whether an ancestral cell of the subject host cell was modified to comprise the polynucleotide sequence.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., a promoter, translational attenuation sequences) can be used to alter or regulate expression of a second native gene or nucleic acid molecule, wherein the expression or regulation of the second native gene or nucleic acid molecule differs from normal expression or regulation in a parent cell.

The term “homologous” or “homolog” refers to a molecule or activity found in or derived from a host cell, species or strain. For example, a heterologous or exogenous nucleic acid molecule can be homologous to a native host cell gene, and can optionally have an altered expression level, a different sequence, an altered activity, or any combination thereof.

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST 2.0 software as defined by Altschul et al. (1997), Nucl. Acids Res. 25:3389-3402, with the parameters set to default values.

### Immunogenic Compositions

In certain aspects, the present disclosure provides immunogenic compositions comprising or consisting of one or more SOX2 peptide antigens disclosed herein. In certain embodiments, the immunogenic composition comprises an isolated peptide or polypeptide comprising or consisting of the amino acid sequence set forth in any one of SEQ ID NOs:2-7. Presently disclosed immunogenic compositions are capable of eliciting an immune response (e.g., production of antigen-specific T cells, antibodies, cytokines, or the like) against a disease or disorder characterized by or otherwise associated with SOX2 expression (including, in certain embodiments, overexpression) and/or activity, such as multiple myeloma. In certain embodiments, a composition comprises any one, two, three, four, five, or six of the amino acid sequences set forth in SEQ ID NOs:2-7; e.g., in a fusion polypeptide and/or as isolated polypeptides each independently being of no more than about 250, no more than about 200, no more than about 150, no more than about 100, no more than about 50, no no more than about 25, no more than about 20, or no more than about 15 amino acids in length. An exemplary immunogenic fusion polypeptide can include two or more of the amino acid sequences set forth in SEQ ID NOs:2-7, in any order, and can include two or more copies of any one or more of the amino acid sequences set forth in SEQ ID NOs:2-7. In some embodiments, a self-cleaving peptide (e.g., P2A, T2A, E2A, F2A) is disposed between two SOX2 peptides of a fusion. In certain embodiments, the immunogenic composition further comprises a pharmaceutically acceptable adjuvant. An adjuvant is intended to enhance (or improve, augment) the immune response to the immunogenic peptides and fusion polypeptides comprising the peptide (i.e., increase the level of the specific immune response to the immunogenic peptide or fusion polypeptide and in a statistically, biologically, or clinically significant manner compared with the level of the specific immune response in the absence of administering the adjuvant).

For administration in humans, a pharmaceutically acceptable adjuvant is one that has been approved or is approvable for human administration by pertinent regulatory bodies. Desired adjuvants augment the response to the immunogenic peptide or fusion polypeptide without causing conformational changes in the immunogen that might adversely affect the qualitative immune response. Suitable adjuvants include aluminum salts, such as alum (potassium aluminum sulfate), or other aluminum containing adjuvants such as aluminum hydroxide, aluminum phosphate, or aluminum sulfate. Other pharmaceutically suitable adjuvants include nontoxic lipid A-related adjuvants such as, by way of non-limiting example, nontoxic monophosphoryl lipid A (see, e.g., Persing et al., Trends Microbiol. 25 10:s32-s37 (2002)), for example, 3 De-O-acylated monophosphoryl lipid A (MPL) (see, e.g., United Kingdom Patent Application No. GB 2220211). Other useful adjuvants include QS21 and QuilA that comprise a triterpene glycoside or saponin isolated from the bark of the Quillaja saponaria Molina tree found in South America (see, e.g., Kensil et al., in Vaccine Design: The Subunit and Adjuvant Approach (eds. Powell and

30 Newman, Plenum Press, NY, 1995); U.S. Pat. No. 5,057,540). Other suitable adjuvants include oil in water emulsions, optionally in combination with immune stimulants, such as monophosphoryl lipid A (see, e.g., Stoute et al., N. Engl. J Med. 336, 86-91 (1997)). Other suitable adjuvants include polymeric or monomeric amino acids such as polyglutamic acid or polylysine, liposomes, and CpG (see, e.g., Klinman, 35 Int. Rev. Immunol. 25(3-4):135-54 (2006); U.S. Pat. No. 7,402,572; European Patent
No. 772 619). Also provided are host cells comprising a heterologous polynucleotide that encodes an immunogenic SOX2 peptide or fusion polypeptide as provided herein. In certain embodiments, a host cell comprises an immune cell, such as a human immune cell. In certain embodiments, a host cell comprises a dendritic cell or a T cell. In certain embodiments, an immunogenic composition or host cell is administered to a subject who is HLA-A:02*01⁺.

In some embodiments, an immunogenic composition comprises: (i) an isolated peptide or polypeptide comprising or consisting of the amino acid sequence of SEQ ID NO:2; (ii) an isolated peptide or polypeptide comprising or consisting of the amino acid sequence of SEQ ID NO:3; (iii) an isolated peptide or polypeptide comprising or consisting of the amino acid sequence of SEQ ID NO:4; (iv) an isolated peptide or polypeptide comprising or consisting of the amino acid sequence of SEQ ID NO:5; (v) an isolated peptide or polypeptide comprising or consisting of the amino acid sequence of SEQ ID NO:6; (vi) an isolated peptide or polypeptide comprising or consisting of the amino acid sequence of SEQ ID NO:7; and/or (vii) a variant of the isolated peptide or polypeptide of any one of (i)-(vi) having one, two, or three amino acid differences as compared to SEQ ID NO:2, 3, 4, 5, 6, or 7, wherein the isolated peptide or polypeptide of any one of (i)-(vii) does not comprise an isolated full-length human SOX2. In certain embodiments, (a) one or more copies of any one of (i)-(vii) and/or (b) one or more of any of (i)-(vii) is/are present in a fusion polypeptide, wherein the fusion polypeptide optionally further comprises the amino acid sequence of a self-cleaving peptide.

In certain embodiments, the immunogenic composition is capable of eliciting an immune response in a subject against cancer cells, wherein, optionally, the cancer cells comprise multiple myeloma cells, glioma cells, neck cancer cells, lung cancer cells, plasma cell leukemia cells, and/or ovarian cancer cells.

In certain embodiments, the immunogenic composition further comprises an adjuvant.

Also provided is an isolated polynucleotide encoding: (i) a peptide or polypeptide comprising or consisting of the amino acid sequence of SEQ ID NO:2; (ii)
a peptide or polypeptide comprising or consisting of the amino acid sequence of SEQ ID NO:3;
   (iii) a peptide or polypeptide comprising or consisting of the amino acid sequence of SEQ ID NO:4; (iv) a peptide or polypeptide comprising or consisting of the amino acid sequence of SEQ ID NO:5; (v) a peptide or polypeptide comprising or consisting of the amino acid sequence of SEQ ID NO:6; (vi) a peptide or polypeptide comprising or consisting of the amino acid sequence of SEQ ID NO:7; and/or (vii) a variant of the peptide or polypeptide of any one of (i)-(vii) having one, two, or three amino acid differences as compared to SEQ ID NO:2, 3, 4, 5, 6, or 7, wherein the polynucleotide is optionally contained in a vector and/or the peptide or polypeptide of any one of (i)-(vii) does not comprise a full-length human SOX2. In certain embodiments, the polynucleotide is codon-optimized for expression in a host cell, wherein the host cell is optionally a dendritic cell or a T cell. Also provided is a host cell comprising the polynucleotide, wherein the polynucleotide is heterologous to the host cell, and wherein the host cell is optionally an immune cell and is further optionally a professional antigen-presenting cell. In some embodiments, the host cell is a dendritic cell or a T cell.

Also provided is a method of eliciting an immune response in a subject against a disease or disorder associated with SOX2 expression or activity, the method comprising administering to the subject a presently disclosed binding protein, binding protein-encoding polynucleotide, vector, binding protein-encoding/expressing host cell, composition, the immunogenic composition, peptide-encoding polynucleotide, and/or peptide-encoding host cell.

Also provided is a method for expanding a population of T cells that bind to a peptide (e.g., a peptide comprised in a peptide:HLA complex) selected from: (i) a peptide comprising or consisting of the amino acid sequence of SEQ ID NO:2; (ii) a peptide comprising or consisting of the amino acid sequence of SEQ ID NO:3; (iii) a peptide comprising or consisting of the amino acid sequence of SEQ ID NO:4; (iv) a peptide comprising or consisting of the amino acid sequence of SEQ ID NO: 5; (v) a peptide comprising or consisting of the amino acid sequence of SEQ ID NO:6; and/or (vi) a peptide comprising or consisting of the amino acid sequence of SEQ ID NO:7, the method comprising contacting a sample comprising one or more T cells that bind to the peptide with the immunogenic composition, peptide-encoding polynucleotide, peptide-encoding host cell, and/or antigen-presenting cells that have been contacted with a peptide or polypeptide comprising or consisting of the amino acid sequence set forth in any one of SEQ ID NOS:2-7.

Also provided is a method for generating and/or isolating T cells, the method comprising contacting a sample comprising T cells, wherein the sample optionally comprises peripheral blood cells, with a disclosed immunogenic composition, peptide-encoding polynucleotide, peptide-encoding host cell, and/or antigen-presenting cells (APCs) that express or have been contacted with a SOX2 antigen comprising or consisting of the amino acid sequence set forth in any one of SEQ ID NOS:2-7, and optionally sorting T cells from other cells in the sample, thereby isolating and/or generating T cells. Also provided is a T cell isolated and/or generated by the method.

### Binding Proteins and Host Cells

In certain aspects, the present disclosure provides binding proteins that are capable of binding (e.g., specifically binding) to a SOX2 antigen, such as in the context of a peptide:HLA complex. In some embodiments, the HLA comprises HLA-A*02:01 and/or the SOX2 antigen comprises or consists of the amino acid sequence set forth in any one of SEQ ID NOs:2-7. Also provided are polynucleotides that encode the binding proteins, and host cells that comprise such a polynucleotide and are capable of expressing the encoded binding protein.

The term “SOX2-specific binding protein” as used herein, refers to a protein or polypeptide (such as, for example, a TCR or antigen-binding domain or fragment thereof, or the antigen-binding domain of a CAR, or a TCR-mimic antibody or antigen-binding domain or fragment thereof) that is capable of binding to a SOX2 peptide antigen:HLA HLA complex, e.g., on a cell surface. In some embodiments, a SOX2-specific binding protein does not bind a protein or polypeptide that does not contain the SOX2 peptide antigen and/or does not bind to an HLA complex comprising such a peptide. A host cell (such as, for example, an immune cell) that encodes and/or expresses a SOX2-specific binding protein of this disclosure (i.e., heterologously or otherwise) is, in some contexts, referred to as a “SOX2-specific” cell.

Binding proteins of this disclosure, such as TCRs, scTvs, scTCRs, CARs, and TCR-mimic antibodies and antigen-binding fragments thereof, will contain a binding domain that is capable of binding to a SOX2 antigen, such as in a SOX2 antigen:HLA complex. A “binding domain” (also referred to as a “binding region” or “binding moiety”), as used herein, refers to a molecule or portion thereof (e.g., peptide, oligopeptide, polypeptide, protein) that possesses the ability to specifically and non-covalently associate, unite, or combine with a target (e.g., an antigenic peptide or peptide:MHC complex). A binding domain includes any naturally occurring, synthetic, semi-synthetic, or recombinantly produced binding partner for a biological molecule, a molecular complex (i.e. complex comprising two or more biological molecules), or other target of interest. Certain binding domains include immunoglobulin variable regions or single chain constructs comprising the same (e.g., single chain TCR (scTCR), scTv, scFv).

In certain embodiments, a binding protein comprises one or more variable domain from an immungolublin superfamily binding protein. In some embodiments, a binding proteim comprises a T cell receptor (TCR) α-chain variable domain (Vα) and/or a TCR β-chain variable domain (Vβ). In some embodiments, a binding proteim comprises one or more variable domain from a TCR-mimic antibody (see e.g. Kurosawa et al., Sci Reports 9:9827 (2019); Trenevska et al. Front. Immunol. (2017) doi.org/10.3389/fimmu.2017.01001; Dahan & Reiter, Expert Rec. Mol. Med. 14 e6 (2012) doi.org/10.1017/erm.2012.2; Chang et al. Exper Opin Biol Ther 16:979-987 (2016) doi.org/10.1080/14712598.2016.1176138); Noy et al., Expert Rev. Anticancer Ther. 5(3):523-236 (2005)).

In some embodiments, a binding protein includes a T cell receptor (TCR) α-polypeptide variable (Vα) domain and a TCR β-polypeptide variable (Vα) domain, wherein the binding protein is capable of binding to a peptide containing a SOX2 antigen:HLA complex, wherein the SOX2 antigen comprises or consists of the amino acid sequence set forth in any one of SEQ ID NOs:2-7.

In any of the herein disclosed embodiments, the HLA comprises HLA-A*02:01.

In some embodiments, binding to a SOX2 peptide antigen:HLA complex comprises specific binding. As used herein in the context of a binding interaction, “specifically binds” or “specific for” refers to an association or union of a binding protein (e.g., TCR receptor, scTv, scTCR, CAR, TCR-mimic antibody) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 101 M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M).

In certain embodiments, a binding protein of the present disclosure binds to a SOX2-containing peptide (or a SOX2 peptide:HLA complex) with a K_{d} of less than about 10⁻⁸ M, less than about 10⁻⁹ M, less than about 10⁻¹⁰ M, less than about 10⁻¹¹ M, less than about 10⁻¹² M, or less than about 10⁻¹³ M, or with an affinity that is about the same as, at least about the same as, or is greater than at or about the affinity exhibited by an exemplary binding protein provided herein, such as any of the exemplary SOX2-specific TCRs provided herein, for example, as measured by the same assay. In certain embodiments, a SOX2 binding protein comprises a SOX2-specific immunoglobulin superfamily binding protein or a binding portion thereof.

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

A variety of assays are known for identifying binding domains of the present disclosure that specifically bind a particular target, as well as determining binding domain or binding protein affinities, such as multimer/tetramer staining (e.g., peptide:MHC tetramer), Western blot, ELISA, analytical ultracentrifugation, spectroscopy and surface plasmon resonance (Biacore®) analysis (see, e.g., Dolton et al., Immunology 146:11-22, 2015, Scatchard et al., Ann. NY Acad. Sci. 51:660, 1949; Wilson, Science 20295:2103, 2002; Wolff et al., Cancer Res. 53:2560, 1993; and U.S. Pat. Nos. 5,283,173, 5,468,614, or the equivalent; all incorporated herein by reference). Binding domains can also be identified by screening e.g. T cells, B cells, plasma cells, PBMCs, or hybridomas for reactivity against/binding to a SOX2 peptide antigen or SOX2 peptide antigen:HLA complex as provided herein. For example, these cells or their supernatants may be exposed to antigen presenting cells that express or have been pulsed with an antigen of interest. Binding proteins can also be raised by introducing an antigen of interest into a suitable host, such as a mouse, rabbit, camel, non-human primate, or shark to which the antigen is foreign, then isolating T cells, NK-T cells, NK cells, B cells, splenocytes, plasma cells, or the like, from the host and determining whether the isolated cells express a binding protein specific for the antigen.

In certain embodiments, a binding protein or fusion protein (e.g., TCR, scTCR, CAR, scTv, TCR-mimic antibody or antigen-binding fragment) of the present disclosure is expressed by a host cell (e.g., by a T cell, NK cell, or NK-T cell heterologously expressing the binding protein), preferably at the cell surface when the binding protein is capable of functioning as a receptor at the cell surface. Avidity of such a host cell for a SOX2 peptide antigen or SOX2 peptide antigen:HLA complex can be determined by, for example, exposing the host cell to the peptide, or to a peptide:HLA complex (e.g., organized as a tetramer), or to an antigen-presenting cell (APC) that presents the peptide to the host cell, optionally in a peptide:HLA complex, and then measuring an activity of the host cell, such as, for example, production or secretion of cytokines (e.g., IFN-γ; TNFα); increased expression of host cell signaling or activation components (e.g., CD137 (4-1BB)); proliferation of the host cell; or killing of the APC (e.g., using a labeled-chromium release assay or a caspase −3/7 assay). Activity of TCR-mimic antibodies can be assessed using standard antibody assays, such as for example ELISA, BLI, SPR, an effector function assay such as using a target cell and an immune effector cell (e.g. expressing an FcTR driving expression of a cell activation reporter element), or the like.

The term “functional avidity” refers to a biological measure or activation threshold of an in vitro immune cell (e.g., T cell, NK cell, NK-T cell) response to a given concentration of a ligand (e.g. antigen), wherein the biological measures can include cytokine production (e.g., IFNγ production, IL-2 production, etc.), cytotoxic activity, activation, and proliferation. For example, T cells that biologically (immunologically) respond in vitro to a low antigen dose by producing cytokines, being cytotoxic, expressing an activation marker, or proliferating are considered to have high functional avidity, while T cells having lower functional avidity require higher amounts of antigen before an immune response, similar to that obtained by the higher-avidity T cells, is elicited. It will be understood that functional avidity is different from affinity and avidity. Affinity refers to the strength of any given bond between a binding protein and its antigen/ligand. Some binding proteins are multivalent and bind to multiple antigens—in this case, the strength of the overall connection is the avidity.

Numerous correlations exist between the functional avidity and the effectiveness of an immune response. Some ex vivo studies have shown that distinct T cell functions (e.g., proliferation, cytokine production (e.g. as measured or detected using e.g. ELISA, Luminex (e.g. Luminex xMAP®), etc.) can be triggered at different thresholds (see, e.g., Betts et al., J. Immunol. 172:6407, 2004; Langenkamp et al., Eur. J. Immunol. 32:2046, 2002). Factors that can affect functional avidity include (a) the affinity of a TCR for the pMHC-complex, that is, the strength of the interaction between the TCR and pMHC (Cawthon et al., J. Immunol. 167:2577, 2001), (b) expression levels of the TCR and the CD4 or CD8 co-receptors, and (c) the distribution and composition of signaling molecules (Viola and Lanzavecchia, Science 273:104, 1996), as well as expression levels of molecules that attenuate T cell function and TCR signaling.

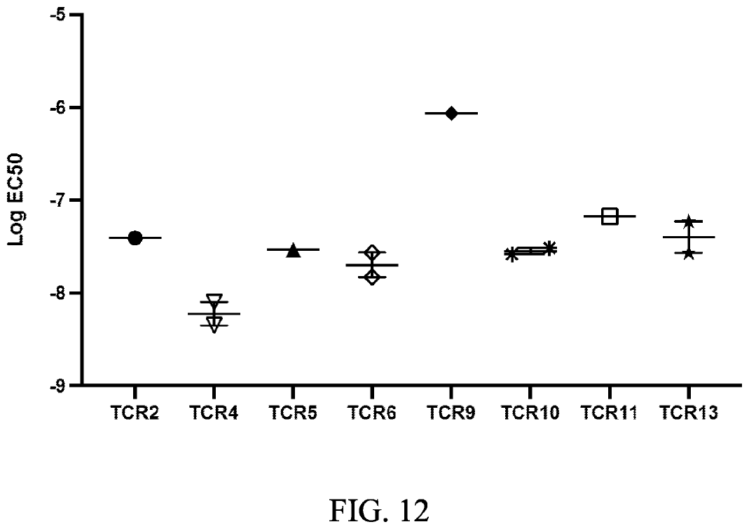

The concentration of antigen needed to induce a half-maximum response between the baseline and maximum response after a specified exposure time is referred to as the “half maximal effective concentration” or “EC₅₀”. The EC₅₀ value is generally presented as a molar (moles/liter) amount, but it is often converted into a logarithmic value as follows −log₁₀(EC₅₀). For example, if the EC₅₀ equals 1 μM (10⁻⁶ M), the log₁₀(EC₅₀) value is −6. Another value used is pEC₅₀, which is defined as the negative logarithm of the EC₅₀ (−log₁₀(EC₅₀)). See, for example FIG. 12. In the above example, the EC₅₀ equaling 1 μM has a pEC50 value of 6. In certain embodiments, the functional avidity of a binding protein of this disclosure will be a measure of its ability to promote IFNγ production by immune cells (e.g., T cells, NK-T cells, NK cells), which can be measured using assays known in the art and/or described herein. “High functional avidity” TCRs or binding domains thereof refer to those TCRs or binding domains thereof having a EC₅₀ of at least 10⁻⁴ M, at least about 10⁻⁵ M, or at least about 10⁻⁶ M.

Also contemplated are fusion proteins comprising TCR or scTCR or scTv variable domains according to the present disclosure linked to a constant domain of an antibody (e.g., IgG (1, 2, 3, 4), IgE, IgD, IgA, IgM, and variants thereof) or a fragment thereof (e.g., a fragment that, in some embodiments, retains binding to one or more Fc receptors, to C1q, to Protein A, to Protein G, or any combination thereof), and including immunoglobulin heavy chain monomers and multimers, such as Fc dimers; see, e.g., Wong et al., J. Immunol. 198:1 Supp. (2017). Variant Fc polypeptides comprising mutations that enhance, reduce, or abrogate binding to or by, e.g., FcRn or other Fc receptors, are known and are contemplated within this disclosure.

An“altered domain” or “altered protein” refers to a motif, region, domain, peptide, polypeptide, or protein with a non-identical sequence identity to a wild type motif, region, domain, peptide, polypeptide, or protein (e.g., a wild type TCRα polypeptide, TCRβ polypeptide, TCRα constant domain, TCRβ constant domain) of at least 85% (e.g., 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%).

In certain embodiments, binding proteins according to the present disclosure comprise variant sequences as compared to a reference or wild-type sequence (e.g., a variant TCR CDR3β as compared to the parent or wild-type TCR CDR3β of a TCR of a T cell as described herein). As used herein, a “variant” amino acid sequence, peptide, or polypeptide, refers to a an amino acid sequence (or peptide or polypeptide) having one or two amino acid substitutions, deletions, or insertions as compared to a reference or wild-type amino acid sequence. In certain embodiments, a variant amino acid sequence, peptide, or polypeptide, retains substantially the same functionality (e.g., binding specificity and affinity for a peptide:HLA complex) as the reference or wild-type molecule; for example, a variant TCR CDR3β as disclosed herein retains about 50%, about 60%, about 70%, about 80%, about 90%, about 95%, about 99%, or 100% of the antigen-binding specificity or affinity as compared to the parent or wild-type TCR CDR3β.

Engineering in TCR variable domain framework regions has been shown to improve protein expression, without reducing or materially reducing binding function, in some instances (Thomas et al., Nature Communications 10:4451 (2019); doi.org/10.1038/s41467-019-12441-w; which framework mutations are incorporated herein by reference). Such mutations can be employed, optionally in combination with cysteine mutations (e.g. T48C (Cα) and S79C or S57C (Cβ)) in the TCR constant regions, to potentially improve expression and function.

Amino acids associated with “dominant” (vs. endogenous TCR) expression of engineered TCRs include:
Vα:
   In framework region 1, a T at IMGT position 5, a Q at IMGT position 8, a V at IMGT position 19, a T at IMGT position 20, and/or a T at IMGT position 24;
   In framework region 2, a L at IMGT position 39, a M at IMGT position 50, and/or a R at IMGT position 55; and
   In framework region 3, a A at IMGT position 66, a S at IMGT position 86, and a L at IMGT position 96.
Vβ:
   In framework region 1, a R at IMGT position 9 and/or a Y at IMGT position 10; and
   In framework region 2, a Q at IMGT position 43.

Thus, introducing one or more of the above amino acids into a TCR V-region at the indicated position(s) (if not already present in the native amino acid sequence) can generate a variant with potentially improved expression and optionally function.

In some embodiments, an isolated binding protein is provided that is capable of binding to a SOX2 peptide:HLA complex, wherein the SOX2 peptide comprises or consists of the amino acid sequence set forth in SEQ ID NO.:5, 2, 3, 4, 6, or 7, and wherein, optionally, the binding comprises specific binding. In certain embodiments, the HLA comprises HLA-A*02:01.

In some embodiments, a binding protein comprises an immunoglobulin superfamily variable domain. In some embodiments, the binding protein comprises a TCR α-chain variable domain (Vα) and/or a TCR β-chain variable domain (Vβ). In some embodiments, the binding protein comprises a heavy chain variable domain (VH) and/or a light chain variable domain (VL) of a TCR-mimic antibody.

In certain embodiments, a binding protein comprises: (i) the amino acid sequence set forth in any one of SEQ ID NOs.:52, 53, 100, 101, 16, 17, 28, 29, 40, 41, 64, 65, 76, 77, 88, 89, 112, 113, 124, 125, 136, 137, 148, and 149, or a variant thereof comprising one, two, or three amino acid substitutions, wherein the amino acid sequence is optionally a CDR3β; (ii) the amino acid sequence set forth in any one of SEQ ID NOs.: 51, 99, 15, 27, 39, 63, 75, 87, 111, 123, 135, and 147, or a variant thereof comprising one, two, or three amino acid substitutions, wherein the amino acid sequence is optionally a CDR2β;

(iii) the amino acid sequence set forth in any one of SEQ ID NOs.: 50, 98, 14, 26, 38, 62, 74, 86, 110, 122, 134, and 146, or a variant thereof comprising one, two, or three amino acid substitutions, wherein the amino acid sequence is optionally a CDR1β; (iv) the amino acid sequence set forth in any one of SEQ ID NOs.: 57, 58, 105, 106, 21, 22, 33, 34, 45, 46, 69, 70, 81, 82, 93, 94, 117, 118, 129, 130, 141, 142, 153, and 154, or a variant thereof comprising one, two, or three amino acid substitutions, wherein the amino acid sequence is optionally a CDR3α; (v) the amino acid sequence set forth in any one of SEQ ID NOs.: 56, 104, 20, 32, 44, 68, 80, 92, 116, 128, 140, and 152, or a variant thereof comprising one, two, or three amino acid substitutions, wherein the amino acid sequence is optionally a CDR2α; and/or (vi) the amino acid sequence set forth in any one of SEQ ID NOs.: 55, 103, 19, 31, 43, 67, 79, 91, 115, 127, 139, and 151, or a variant thereof comprising one, two, or three amino acid substitutions, wherein the amino acid sequence is optionally a CDR1α.

In some embodiments, a binding protein comprises (1) a TCR Vα comprising CDR1α, CDR2α, and CDR3α; and (2) a TCR Vβ comprising CDR1β, CDR2β, and CDR3β, wherein the CDR1α, CDR2α, CDR3α, CDR1β, CDR2β, and CDR3β are as set forth in: (i) SEQ ID NOs.:55, 56, 57 or 58, 50, 51, and 52 or 53, respectively; (ii) SEQ ID NOs.:103, 104, 105 or 106, 98, 99, and 100 or 101, respectively; (iii) SEQ ID NOs.:19, 20, 21 or 22, 14, 15, and 16 or 17, respectively; (iv) SEQ ID NOs.:31, 32, 33 or 34, 26, 27, and 28 or 29, respectively; (v) SEQ ID NOs.:43, 44, 45 or 46, 38, 39, and 40 or 41, respectively; (vi) SEQ ID NOs.:67, 68, 69 or 70, 62, 63, and 64 or 65, respectively; (vii) SEQ ID NOs.:79, 80, 81 or 82, 74, 75, and 76 or 77, respectively; (viii) SEQ ID NOs.:91, 92, 93 or 94, 86, 87, and 88 or 89, respectively; (ix) SEQ ID NOs.:115, 116, 117 or 118, 110, 111, and 112 or 113, respectively; (x) SEQ ID NOs.:127, 128, 129 or 130, 122, 123, and 124 or 125, respectively; (xi) SEQ ID NOs.:139, 140, 141 or 142, 134, 135, and 136 or 137 respectively; or (xii) SEQ ID NOs.:151, 152, 153 or 154, 146, 147, and 148 or 149, respectively.

In some embodiments, a Vα comprises or consists of an amino acid sequence having at least 80%, at least 85%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.: 54, 102, 18, 30, 42, 66, 78, 90, 114, 126, 138, and 150. In some embodiments, a Vβ comprises or consists of the amino acid sequence having at least 80%, at least 85%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.:49, 97, 13, 25, 37, 61, 73, 85, 109, 121, 133, and 145.

In certain embodiments, a binding protein comprises a Vα and a Vβ that comprise or consist of amino acid sequences having at least 80%, at least 85%, at least 90%, at least 91%, at least 92%, at least 93%, at least 94%, at least 95%, at least 96%, at least 97%, at least 98%, or at least 99% identity to the amino acid sequences set forth in: (i) SEQ ID NOs.:54 and 49, respectively; (ii) SEQ ID NOs.:102 and 97, respectively; (iii) SEQ ID NOs.:18 and 13, respectively; (iv) SEQ ID NOs.:30 and 25, respetively; (v) SEQ ID NOs.:42 and 37, respectively; (vi) SEQ ID NOs.:66 and 61, respectively; (vii) SEQ ID NOs.:78 and 73, respectively; (viii) SEQ ID NOs.:90 and 85, respectively; (ix) SEQ ID NOs.:114 and 109, respectively; (x) SEQ ID NOs.:126 and 121, respectively; (xi) SEQ ID NOs.:138 and 133 respectively; or (xii) SEQ ID NOs.:150 and 145, respectively.

In some embodiments, a binding protein is provided that comprises a Vα and a Vβ that comprise or consist of the amino acid sequences according to: (i) SEQ ID NOs.:54 and 49, respectively; (ii) SEQ ID NOs.:102 and 97, respectively; (iii) SEQ ID NOs.:18 and 13, respectively; (iv) SEQ ID NOs.:30 and 25, respetively; (v) SEQ ID NOs.:42 and 37, respectively; (vi) SEQ ID NOs.:66 and 61, respectively; (vii) SEQ ID NOs.:78 and 73, respectively; (viii) SEQ ID NOs.:90 and 85, respectively; (ix) SEQ ID NOs.:114 and 109, respectively; (x) SEQ ID NOs.:126 and 121, respectively; (xi) SEQ ID NOs.:138 and 133 respectively; or (xii) SEQ ID NOs.:150 and 145, respectively.

In some embodiments, a binding protein is comprised in an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:48, 96, 12, 24, 36, 60, 72, 84, 108, 120, 132, and 144.

In certain embodiments, a binding protein comprises a TCR Vα domain and a TCR Vβ domain, comprising: (i) a CDR3β according to a TCR of a T cell from: Donor 1 Line 1, Donor 1 Line 3, Donor 2 Line 8, Donor 2 Line 1, Donor 2 Line 13, Donor 2 Line 11, or Donor 2 Line 19; a (ii) CDR3α according to a TCR of a T cell from: Donor 1 Line 1, Donor 1 Line 3, Donor 2 Line 8, Donor 2 Line 1, Donor 2 Line 13, Donor 2 Line 11, or Donor 2 Line 19; (iii) a Vα domain having at least 90% (90%, 91%, 92%, 93%, 94%, 95%, 96%, 97%, 98%, 99%, or more) amino acid identity to the Vα domain of a TCR of a T cell from: Donor 1 Line 1, Donor 1 Line 3, Donor 2 Line 8, Donor 2 Line 1, Donor 2 Line 13, Donor 2 Line 11, or Donor 2 Line 19; or (iv) a Vβ domain having at least 90% (90%, 91%, 92%, 93%, 94%, 95%, 96%, 97%, 98%, 99%, or more) amino acid identity to the Vβ domain of a TCR of a T cell from: Donor 1 Line 1, Donor 1 Line 3, Donor 2 Line 8, Donor 2 Line 1, Donor 2 Line 13, Donor 2 Line 11, or Donor 2 Line 19, provided that (a) at least three or four of the CDRs have no mutations; (b) the CDRs that do have mutations have only up to two amino acid substitutions, up to a contiguous five amino acid deletion, or a combination thereof, and (c) the encoded binding protein retains its ability to bind to a peptide comprising a SOX2:HLA complex, e.g., a SOX2 peptide according to SEQ ID NO:2, 3, 4, 5, 6, or 7, in complex with an HLA-A*02:01 molecule.

In further embodiments the binding protein further comprises a CDR1β, a CDR2β, a CDR1α, and/or a CDR2α according to TCR of a T cell from: Donor 1 Line 1, Donor 1 Line 3, Donor 2 Line 8, Donor 2 Line 1, Donor 2 Line 13, Donor 2 Line 11, or Donor 2 Line 19.

TCRs and T cell clones of the presently disclosed SOX2-specific T cell lines are identifiable, and have sequences which are determinable, using known methods. See, e.g., Bleakley et al., Blood 115:4923-4933, 2010; Warren et al., Blood 91(6):2197-2207 (1998); Walter et al., N. Engl. J. Med. 333(16):1038-1044; PCT Publication No. WO 2018/058002 (Example 1), which methods and related reagents are incorporated herein by reference. For example, full TCR regions can be identified using 5′ first-strand complementary DNA (cDNA) amplification and rapid amplification of cDNA ends polymerase chain reaction (RACE-PCR) using a SMARTer RACE cDNA Amplification Kit (Clontech Laboratories). Briefly, cDNA is synthesized from RNA using 5′ CDS Primer A, SMARTer IIA oligo, and SMARTScribe Reverse Transcriptase. The cDNA is then used to perform a RACE-PCR reaction using Phusion High-Fidelity DNA Polymerase and gene-specific primers for the TCR α (hTCR_Calpha-R 5′-CAGCCGCAGCGTCATGAGCAGATTA-3′(SEQ ID NO:8)) or TCR β chain (hTCR_Cb1-R 5′-CCACTTCCAGGGCTGCCTTCAGAAATC-3′ (SEQ ID NO:9) and hTCR_Cb2-R 5′-TGGGATGGTTTTGGAGCTAGCCTCTGG-3′ (SEQ ID NO:10)). RACE-PCR products are purified and sequenced to identify TCR α and β polypeptides. TCR variable, diversity, and joining regions can be defined using IMGT/V-QUEST software.

TCRs can be constructed by pairing the TRA and TRB sequences encoding the dominant polypeptides in each SOX2/HiLA-A*02:01-specific T cell clone. TRA and TRB sequences are confirmed by PCR using a forward primer from the 5′ end of the appropriate V region and reverse primers from the TRA or TRB constant region(s), followed by Sanger sequencing.

In any of the herein disclosed embodiments, the binding protein comprises a TCR, a single-chain TCR (scTCR), a scTCR, a scTv, a chimeric antigen receptor (CAR), or any combination thereof. Examples of TCRs of the present disclosure include TCR1, TCR2, TCR1, TCR2, TCR4, TCR5, TCRδ, TCR7, TCR9, TCR10, TCR11, TCR13, TCR15, and TCR16 (alternatively referred to as SOX2 TCR #01, SOX2 TCR #02, SOX2 TCR #04, SOX2 TCR #05, SOX2 TCR #06, SOX2 TCR #07, SOX2 TCR #09, SOX2 TCR #10, SOX2 TCR #11, SOX2 TCR #13, SOX2 TCR #15, and SOX2 TCR #16, respectively. Amino acid sequences of these TCRs are provided herein.

Methods for producing engineered TCRs are described in, for example, Bowerman et al., Mol. Immunol., 46(15):3000 (2009), the techniques of which are herein incorporated by reference. Methods for making CARs are known and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., 2007, Clin. Cancer Res. 13:5426, the techniques of which are herein incorporated by reference.

In certain embodiments, a SOX2-specific binding domain alone (i.e., without any other portion of the binding protein) can be soluble and can bind to the antigen or antigen:HLA complex with a K_{d} of less than about 108 M, less than about 10⁻⁹ M, less than about 10⁻¹⁰ M, less than about 10⁻¹¹ M, less than about 10⁻¹² M, or less than about 10⁻¹³ M. In particular embodiments, a SOX2-specific binding domain includes an antigen-specific scTCR (e.g., single chain αβTCR proteins such as Vα-L-Vβ, VP-L-Vα, Vα-Cα-L-Vα, or Vα-L-VP-Cβ, wherein Vα and Vβ are TCRα and β variable domains respectively, Ca and CP are TCRα and β constant domains, respectively, and L is a linker).

In certain embodiments, the binding protein further comprises a TCR β polypeptide constant domain (Cβ), a TCR α polypeptide constant domain (Cα), or both. A Vβ and a Cβ together comprise TCR β polypeptide or chain. A Vα and a Cα together comprise TCR α polypeptide or chain. In some embodiments, the Cβ comprises or consists of an amino acid sequence having at least at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NO.:156 or 157, and/or the Cα comprises or consists of an amino acid sequence having at least at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NO.:155.

In certain embodiments, the Cβ and/or the Cα comprises one or more non-native amino acid at a position such that when the the Cβ and the Cα associate to form a dimer, a non-native disulfide bond is formed between the Cβ and the Cα, wherein, optionally, the non-native amino acid comprises a cysteine in the Cβ and/or a cysteine in the Cα. In some embodiments, the binding protein comprises a TCR Cβ and a TCR Cα, wherein the TCR Cβ comprises a cysteine amino acid in place of a native serine at amino acid position 57 (e.g., GV(S→C)TD) and the TCR Cα comprises a cysteine amino acid in place of a native threonine at amino acid position 48 (e.g., DK(T→C)VL; see. e.g., Cohen et al., Cancer Res. 67(8):3898-3903 (2007)).

In certain embodiments, a binding protein comprises two components, such as a polypeptide and a β polypeptide, which can associate on the cell surface to form a functional binding protein. The two associated components may comprise mature proteins. In certain embodiments, an antigen-binding fragment of a TCR comprises a single chain TCR (scTCR), which comprises both the TCR V_{α} and TCR V_{β} domains, but only a single TCR constant domain (C_{α} or C_{β}).

In further embodiments, an antigen-binding fragment of a TCR or a chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

Binding proteins of the present disclosure can be expressed by a host cell, e.g. whether as a cell surface molecule (e.g. TCR, scTCR, CAR) or a soluble molecule (e.g. TCR-mimic antibody, scTv (see e.g. Novoty et al. PNAS88(19):8646-8650 (1991)).

A host cell (e.g., immune cell) of the present disclosure may comprise a single polynucleotide that encodes a binding protein as described herein, or the binding protein may be encoded by more than one polynucleotide. In other words, components or portions of a binding protein may be encoded by two or more polynucleotides, which may be contained on a single nucleic acid molecule or may be contained on two or more nucleic acid molecules.

In certain embodiments, a polynucleotide encoding two or more components or portions of a binding protein of the present disclosure comprises the two or more coding sequences operatively associated in a single open reading frame. Such an arrangement can advantageously allow coordinated expression of desired gene products, such as, for example, contemporaneous expression of alpha- and beta-polypeptides of a TCR, such that they are produced in about a 1:1 ratio. In certain embodiments, two or more substituent gene products of a binding protein of this disclosure, such as a TCR (e.g., alpha- and beta-chains) or a TCR-mimic antibody (e.g., heavy and light chains), are expressed as separate molecules and associate post-translationally. In further embodiments, two or more substituent gene products of a binding protein of this disclosure are expressed as a single peptide with the parts separated by a cleavable or removable segment. For instance, self-cleaving peptides useful for expression of separable polypeptides encoded by a single polynucleotide or vector are known in the art and include, for example, a Porcine teschovirus-1 2A (P2A) peptide, a Thoseaasigna virus 2A (T2A) peptide, an Equine rhinitis A virus (ERAV) 2A (E2A) peptide, and a Foot-and-Mouth disease virus 2A (F2A) peptide.

In certain embodiments, a binding protein of the present disclosure comprises one or more junction amino acids. “Junction amino acids” or “junction amino acid residues” refer to one or more (e.g., 2 to about 10) amino acid residues between two adjacent motifs, regions or domains of a polypeptide, such as between a binding domain and an adjacent constant domain or between a TCβ chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein of this disclosure (e.g., a P2A peptide disposed between a TCR α-polypeptide and a TCR β-polypeptide, the self-cleavage of which can leave one or more junction amino acids in the α-polypeptide, the TCR β-polypeptide, or both).

In any of the embodiments described herein, an encoded polypeptide of this disclosure can comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides.

In any of the herein disclosed embodiments, a polynucleotide encoding a binding protein (and, optionally, accessory protein or proteins) can be codon optimized for expression in a host cell. Once a coding sequence is known or identified, codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized.

Any suitable host cell cell may encode a binding protein of this disclosure, or be engineered to include a heterologous polynucleotide encoding a binding protein of this disclosure. In some embodiments, an immune cell is preferred (e.g., a T cell, a NK cell, a NK-T cell, a B cell, or a plasma cell). In some embodiments, an immune cell comprises a CD4⁺ T cell, a CD8⁺ T cell, or both. Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein.

In certain embodiments, polynucleotides encoding binding proteins of this disclosure, e.g., TCRs, can be codon optimized to enhance expression in a particular host cell, such as a cell of the immune system, a hematopoietic stem cell, a T cell, a primary T cell, a T cell line, a NK cell, or a natural killer T cell. Exemplary T cells include CD4⁺ T cells, CD8⁺ T cells, and related subpopulations thereof (e.g., naïve, central memory, effector memory, stem cell memory).

Any appropriate method can be used to transfect or transduce the cells, for example, the T cells, or to administer the polynucleotides or compositions of the present methods. Known methods for delivering polynucleotides to host cells include, for example, use of cationic polymers, lipid-like molecules, and certain commercial products such as, for example, IN-VIVβ-JET PEI. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein. Other methods of introducing a binding protein-encoding polynucleotide into a host cell or host cell genome include gene engineering strategies such as e.g., CRISPR/Cas systems.

In any of the herein disclosed embodiments, the immune cell comprises a T cell, a NK cell, a NK-T cell, or any combination thereof. In certain embodiments, the immune cell comprises a CD8⁺ T cell, a CD4⁺ T cell, or both. In certain embodiments, a host cell (e.g. immune cell, such as a T cell (e.g., CD8⁺ T cell), that expresses a SOX2-specific binding protein of this disclosure) is capable of producing a cytokine when in the presence of a SOX2 peptide antigen according to any one or more of SEQ ID NOs:2-7. In certain embodiments, the cytokine is or comprises IFN-γ. In certain embodiments, host cell produces a cytokine when the SOX2 peptide antigen is present at about 1 ng/mL, about 10 ng/mL, about 100 ng/mL, or about 1000 ng/mL. In some embodiments, a target or antigen-presenting cell cell that is capable of processing and presenting the SOX2 peptide antigen in a peptide:HLA complex is further present.

In certain embodiments, a host cell (e.g. an immune cell, such as a T cell) that expresses a SOX2-specific binding protein of this disclosure is capable of expanding when in the presence of a SOX2 peptide antigen (and optionally an antigen-presenting cell such as a dendritic cell or a T2 cell) according to any one or more of SEQ ID NOs:2-7, and optionally in the further presence of a cytokine. In certain embodiments, the cytokine is or comprises IFN-γ. In certain embodiments, the SOX2 peptide antigen is present at about 1 ng/mL, about 10 ng/mL, about 100 ng/mL, or about 1000 ng/mL.

In certain embodiments, a host cell (e,g, an immune cell, such as a T cell) that expresses a SOX2-specific binding protein of this disclosure has increased expression of CD137 when co-cultured (e.g., in a 12-hour cell culture) with a multiple myeloma cell. In certain embodiments, the multiple myeloma cell is a L363 cell (DSMZ No. ACC 49). In certain embodiments, the multiple myeloma cell is patient-derived.

In certain embodiments, a host cell (e.g. an immune cell, such as a T cell) that expresses a SOX2-specific binding protein of this disclosure is capable of specifically killing a multiple myeloma cell (e.g., in vitro, ex vivo, or in vivo). In certain embodiments, the multiple myeloma cell is a L363 cell (DSMZ No. ACC 49). In certain embodiments, the multiple myeloma cell is patient-derived.

In some embodiments, a host cell produces IFN-γ when in the presence of the SOX2 antigen:HLA complex, wherein, optionally, the SOX2 antigen:HLA complex is expressed on the surface of a target cell. In certain embodiments, the binding protein is capable of binding to the YLPGAEVPEPA (SEQ ID NO.:5):HLA complex with an IFNγ production pEC₅₀ of between 6.0 and 9.0 (i.e. including 6.0, 9.0, and any value therebetween), between 6.0 and 8.5, between 6.0 and 8.0, between 6.0 and 7.5, between 6.0 and 7.0, between 6.0 and 6.5, between 6.5 and 9.0, between 6.5 and 8.5, between 6.5 and 8.0, between 6.5 and 7.5, between 6.5 and 7.0, between 7.0 and 9.0, between 7.0 and 8.5, between 7.0 and 8.0, between 7.0 and 7.5, between 7.5 and 9.0, between 7.5 and 8.5, between 7.5 and 8.0, between 8.0 and 9.0, between 8.0 and 8.5, or between 8.2 and 9.0.

In some embodiments of a host cell, the binding protein is capable of binding to the YLPGAEVPEPA (SEQ ID NO.:5):HLA complex with an IFNγ production pEC₅₀ of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0.

In some embodiments of a host cell, the binding protein is capable of binding to the YLPGAEVPEPA (SEQ ID NO.:5):HLA complex with an IFNγ production pEC₅₀ of 6.0 or higher. In some embodiments of a host cell, the binding protein is capable of binding to the YLPGAEVPEPA (SEQ ID NO.:5):HLA complex with an IFNγ production pEC₅₀ of 6.5 or higher. In some embodiments of a host cell, the binding protein is capable of binding to the YLPGAEVPEPA (SEQ ID NO.:5):HLA complex with an IFNγ production pEC₅₀ of 7.0 or higher. In some embodiments of a host cell, the binding protein is capable of binding to the YLPGAEVPEPA (SEQ ID NO.:5):HLA complex with an IFNγ production pEC₅₀ of 7.5 or higher. In some embodiments of a host cell, binding protein is capable of binding to the YLPGAEVPEPA (SEQ ID NO.:5):HLA complex with an IFNγ production pEC₅₀ of 8.0 or higher. In some embodiments, a host cell expresses CD137 when in the presence of cells of any one or more of the following tumor cell lines: CFPAC1, H441, Panc08.13, SW620, SW527, L363, MM1R expressing HLA-A2, and INA6 expressing HLA-A2, wherein, optionally, CD137 expression is assessed by flow cytometry of the host cell following incubation of the host cell with the one or more cells of the tumor cell line or lines. In some embodiments, of a plurality of the host cells present in a sample, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, or 80% or more of the plurality of the host cells are positive for expression of CD137 following an incubation with any one or more of the following tumor cell lines: CFPAC1, H441, Panc08.13, SW620, SW527, L363, MM1R expressing HLA-A2, INA6 expressing HLA-A2. In some embodiments, the incubation comprises a duration of about 16 hours to about 18 hours, optionally wherein the incubation comprises a duration of between 16 and 18 hours. In certain further embodiments, prior to the incubation, the cells of the tumor cell line were administered an agent to increase HLA-A2 expression in the cells of the tumor cell line, wherein, optionally, the agent comprises IFN-γ.

In any of the foregoing embodiments, a host cell (e.g., immune cell such as a T cell, NK-T cell, or NK cell) may be modified to reduce or eliminate expression of one or more endogenous genes that encode a polypeptide involved in immune signaling or other related activities. Examples of gene knockouts include those that encode PD-1, LAG-3, CTLA4, TIM3, an HLA molecule, a TCR component, or the like. Without wishing to be bound by theory, certain endogenously expressed immune cell proteins may be recognized as foreign by an allogeneic host receiving the modified immune cells, which may result in elimination of the modified immune cells (e.g., an HLA allele), or may downregulate the immune activity of the modified immune cells (e.g., PD-1, LAG-3, CTLA4), or may interfere with the binding activity of a heterologously expressed binding protein of the present disclosure (e.g., an endogenous TCR of a modified T cell that binds a non-SOX2 antigen and thereby interferes with the modified T cell binding a cell that expresses a SOX2 antigen or initiating a T cell response to the SOX2 antigen, or contributes to or causes the T cell to become exhausted).

Accordingly, decreasing or eliminating expression or activity of such endogenous genes or proteins can improve the activity, tolerance, or persistence of the host cells in an autologous or allogeneic host setting, and may allow for universal administration of the cells (e.g., to any recipient regardless of HLA type). In certain embodiments, a host cell is a donor cell (e.g., allogeneic) or an autologous cell. In certain embodiments, a host cell of this disclosure comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or α2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013), the gene-editing techniques, compositions, and adoptive cell therapies of which are herein incorporated by reference in their entirety).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

Alternative Cas nucleases may be used, including but not limited to, Cas 12, Cas 13, and Cas 14 nucleases, and variants thereof. For example, Cas nucleases disclosed in WO 2019/178427, which is hereby incorporated by reference in its entirety (including the Cas nucleases, CRISPR-Cas systems, and related methods disclosed therein), may be utilized.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host cell.

A chromosomal gene knockout can be confirmed directly by DNA sequencing of the host cell following use of the knockout procedure or agent. Chromosomal gene knockouts can also be inferred from the absence of gene expression (e.g., the absence of an mRNA or polypeptide product encoded by the gene) following the knockout.

In some embodiments, a polynucleotide encoding a binding protein is heterologous to the host cell and is comprised in an endogenous TCR gene locus of the host cell.

In another aspect, compositions are provided herein that comprise (e.g. an effective amount of) a binding protein, polynucleotide, vector, or host cell of the present disclosure, and a pharmaceutically acceptable carrier, diluent, or excipient.

Also provided herein are unit doses that comprise an effective amount of a modified immune cell or of a composition comprising the host cell. In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less then about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% engineered CD4⁺ T cells, combined with (ii) a composition comprising at least about 70% engineered CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

It will be appreciated that a unit dose of the present disclosure may comprise a host cell as described herein (i.e., encoding and optionally expressing a binding protein specific for a SOX2 antigen) and a cell (e.g., an immune cell such as T cell, NK-T cell, or NK cell) expressing a binding protein specific for a different antigen (e.g., a different SOX2 antigen, or an antigen from a different protein or target, such as, for example, BCMA, CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gpl30, Lewis A, Lewis Y, TNFR1, TNFR2, PD1, PD-L1, PD-L2, HVEM, MAGE-A (e.g., including MAGE-A1, MAGE-A3, and MAGE-A4), mesothelin, NY-ESO-1, PSMA, RANK, ROR1, TNFRSF4, CD40, CD137, TWEAK-R, HLA, tumor- or pathogen-associated peptide bound to HLA, hTERT peptide bound to HLA, tyrosinase peptide bound to HLA, WT-1 peptide bound to HLA, LTOR, LIFRβ, LRP5, MUC1, OSMRβ, TCRα, TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCH1, WT-1, HA¹-H, Robol, α-fetoprotein (AFP), Frizzled, OX40, PRAME, and SSX-2. or the like). For example, a unit dose can comprise modified CD8⁺ T cells expressing a binding protein that specifically binds to a SOX2-HLA complex and modified CD4⁺ T cells (and/or modified CD8⁺ T cells) expressing a binding protein (e.g., a CAR) that specifically binds to a CD19 antigen. It will also be appreciated that any of the immune cells disclosed herein may be administered in a combination therapy.

In any of the embodiments described herein, a unit dose comprises equal, or approximately equal numbers of engineered CD45RA⁻ CD3⁺CD8⁺ and modified CD45RA⁻ CD3⁺CD4⁺T_{M} cells.

Also provided are host cells for expressing a TCR-mimic antibody or antigen-binding fragment according to the present disclosure, as well as host cells that comprise or containing a vector or polynucleotide encoding a TCR-mimic an antibody or antigen-binding fragment.

Examples of such cells include but are not limited to, eukaryotic cells, e.g., yeast cells, animal cells, insect cells, plant cells; and prokaryotic cells, including E. coli. In some embodiments, the cells are mammalian cells. In certain embodiments, the cells are B cells (e.g., immortalized and/or engineered to encode the antibody or antigen-binding fragment), plasma cells, or hematopoietic progenitor cells. In certain embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. . K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, a cell may be transfected with a vector according to the present description. Transfection can be accomplished using methods such as, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody, or an antigen-binding fragment thereof, according to the present disclosure. In such embodiments, the cells may be stably transfected width the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glysocylation or fucosylation) on the antibody or antigen-binding fragment that is not present in a native state of the antibody or antigen-binding fragment (or in a native state of a parent antibody from which the antibody or antigen binding fragment was engineered or derived). Such a PTM may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a CHO cell can comprise a more post-translational modification that is distinct from the antibody when isolated from the human and/or produced by the native human B cell or plasma cell).

Insect cells useful expressing an antibody or antigen-binding fragment include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody or antigen-binding fragment of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

### Polynucleotides, Transgenes and Vectors

In further aspects, the present disclosure provides an isolated polynucleotide that encodes an immunogenic peptide, fusion polypeptide, or binding protein as described herein (e.g., a SOX2-specific TCR, scTv, TCR-mimic antibody or antigen-binding fragment thereof, scTCR, or CAR that comprises TCR V. and Vs domains as described herein (and optionally further comprises constant domains or other components as described herein)).

In certain embodiments, a polynucleotide encoding two or more components or portions of a binding protein of the present disclosure comprises the two or more coding sequences operatively associated in a single open reading frame. Such an arrangement can advantageously allow coordinated expression of desired gene products, such as, for example, contemporaneous expression of alpha- and beta-chains of a TCR, such that they are produced in about a 1:1 ratio. In certain embodiments, two or more substituent gene products of a binding protein of this disclosure, such as a TCR (e.g., alpha- and beta-chains), are expressed as separate molecules and associate post-translationally. In further embodiments, two or more substituent gene products of a binding protein of this disclosure are expressed as a single peptide with the parts separated by a cleavable or removable segment. For instance, self-cleaving peptides useful for expression of separable polypeptides encoded by a single polynucleotide or vector are known in the art and include, for example, a Porcine teschovirus-1 2A (P2A) peptide, a Thoseaasigna virus 2A (T2A) peptide, an Equine rhinitis A virus (ERAV) 2A (E2A) peptide, and a Foot-and-Mouth disease virus 2A (F2A) peptide.

In some embodiments, a polynucleotide comprises DNA, RNA (optionally mRNA), or both. In certain embodiments, a polynucleotide comprises DNA.

In certain embodiments, (1) the polynucleotide encodes an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:48, 96, 12, 24, 36, 60, 72, 84, 108, 120, 132, and 144; and/or (2) the polynucleotide comprises a polynucleotide having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprises or consists of, the nucleotide acid sequence set forth in any one of SEQ ID NOs.: 47, 95, 11, 23, 35, 47, 59, 71, 83, 107, 119, 131, and 143.

In some embodiments, a polynucleotide is codon-optimized for expression in a host cell, wherein, optionally, the host cell comprises an immune system cell, wherein, further optionally, the immune system cell comprises a T cell, a NK-T cell, or a NK cell.

A host cell of the present disclosure may comprise a single polynucleotide that encodes a binding protein as described herein, or the binding protein may be encoded by more than one polynucleotide. In other words, components or portions of a binding protein may be encoded by two or more polynucleotides, which may be contained on a single nucleic acid molecule or may be contained on two or more nucleic acid molecules.

In further embodiments, a binding protein is expressed as part of a transgene construct that encodes one or more additional accessory protein, such as a safety switch protein, a tag, a selection marker, a CD8 co-receptor β-chain, α-chain or both, or any combination thereof. Polynucleotides and transgene constructs useful for encoding and expressing binding proteins and accessory components (e.g., one or more of a safety switch protein, a selection marker, CD8 co-receptor β-chain, or a CD8 co-receptor α-chain).

In certain embodiments, a safety switch protein can be targeted using a cognate drug or other compound to selectively modulate the activity (e.g., lessen or ablate) of such cells when desirable. Safety switch proteins used in this regard include, for example, a truncated EGF receptor polypeptide (huEGFRt) that is devoid of extracellular N terminal ligand binding domains and intracellular receptor tyrosine kinase activity but retains the native amino acid sequence, type I transmembrane cell surface localization, and a conformationally intact binding epitope for pharmaceutical-grade anti-EGFR monoclonal antibody, cetuximab (Erbitux) tEGF receptor (tEGFr; Wang et al., Blood 118:1255-1263, 2011), a caspase polypeptide (e.g., iCasp9; Straathof et al., Blood 105:4247-4254, 2005; Di Stasi et al., N. Engl. J. Med. 365:1673-1683, 2011; Zhou and Brenner, Exp. Hematol. pii:S0301-472X(16)30513-6. doi:10.1016/j.exphem.2016.07.011), RQR8 (Philip et al., Blood 124:1277-1287, 2014), a 10 amino acid tag of the human c-myc protein (Myc) (Kieback et al., Proc. Natl. Acad. Sci. USA 105:623-628, 2008), as discussed herein, and a marker/safety switch polypeptide, such as RQR (CD20+CD34; Philip et al., 2014).

Other accessory components useful for therapeutic cells comprise a tag or selection marker that allows the cells to be identified, sorted, isolated, enriched, or tracked. For example, marked immune cells having desired characteristics (e.g., an antigen-specific TCR and a safety switch protein) can be sorted away from unmarked cells in a sample and more efficiently activated and expanded for inclusion in a therapeutic product of desired purity.

As used herein, the term “selection marker” comprises a nucleic acid construct that confers an identifiable change to a cell permitting detection and positive selection of immune cells transduced with a polynucleotide comprising a selection marker. RQR is a selection marker that comprises a major extracellular loop of CD20 and two minimal CD34 binding sites. In some embodiments, an RQR-encoding polynucleotide comprises a polynucleotide that encodes the 16 amino acid CD34 minimal epitope. In some embodiments, such as certain embodiments provided in the examples herein, the CD34 minimal epitope is incorporated at the amino terminal position of the CD8 stalk domain (Q8). In further embodiments, the CD34 minimal binding site sequence can be combined with a target epitope for CD20 to form a compact marker/suicide gene for T cells (RQR8) (Philip et al., 2014, incorporated by reference herein). This construct allows for the selection of immune cells expressing the construct, with for example, CD34 specific antibody bound to magnetic beads (Miltenyi) and that utilizes clinically accepted pharmaceutical antibody, rituximab, that allows for the selective deletion of a transgene expressing engineered T cell (Philip et al., 2014).

Further exemplary selection markers also include several truncated type I transmembrane proteins normally not expressed on T cells: the truncated low-affinity nerve growth factor, truncated CD19, and truncated CD34 (see for example, Di Stasi et al., N. Engl. J. Med. 365:1673-1683, 2011; Mavilio et al., Blood 83:1988-1997, 1994; Fehse et al., Mol. Ther. 1:448-456, 2000; each incorporated herein in their entirety). One feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non signaling domains or various proteins (e.g., CD19, CD34, LNGFR) also can be employed. Any selection marker may be employed and should be acceptable for Good Manufacturing Practices. In certain embodiments, selection markers are expressed with a polynucleotide that encodes a gene product of interest (e.g., a binding protein of the present disclosure, such as a TCR or CAR). Further examples of selection markers include, for example, reporters such as GFP, EGFP, β-gal or chloramphenicol acetyltransferase (CAT). In certain embodiments, a selection marker, such as, for example, CD34 is expressed by a cell and the CD34 can be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34. In certain embodiments, a selection marker comprises an RQR polypeptide, a truncated low-affinity nerve growth factor (tNGFR), a truncated CD19 (tCD19), a truncated CD34 (tCD34), or any combination thereof.

Polynucleotides encoding a binding protein of the present disclosure, as well as host cells that comprise the same, may, in certain embodiments, further comprise a polynucleotide encoding a CD8 co-receptor protein, or a beta-chain or alpha-chain component thereof. By way of background, inclusion of CD4⁺ T cells in an immunotherapy cell product can provide antigen-induced IL-2 secretion and augment persistence and function of transferred cytotoxic CD8⁺ T cells (see, e.g., Kennedy et al., Immunol. Rev. 222:129 (2008); Nakanishi et al., Nature 462(7272):510 (2009)). In certain circumstances, a class I-restricted TCR in a CD4⁺ T cells may require the transfer of a CD8 co-receptor to enhance sensitivity of the TCR to class I HLA peptide complexes. CD4 co-receptors differ in structure to CD8 and cannot effectively substitute for CD8 co-receptors (see, e.g., Stone & Kranz, Front. Immunol. 4:244 (2013); see also Cole et al., Immunology 137(2):139 (2012). Thus, another accessory protein for use in the compositions and methods of this disclosure comprises a CD8 co-receptor or component thereof.

An encoded CD8 co-receptor includes, in some embodiments, a β-chain (see, e.g., UniProtKB identifiers P10966-1, P10966-2, P10966-3, P10966-4, P10966-6, P10966-7, P10966-8, and P10966-9). In further embodiments, the encoded CD8 co-receptor is a recombinant CD8 co-receptor further comprising a RQR polypeptide. Without wishing to be bound by theory, it is believed that distance from the host cell surface is important for RQR polypeptides to function as selection markers/safety switches (Philip et al., 2010 (supra)). In some embodiments, the encoded RQR polypeptide is contained in a β-chain, an α-chain, or both, of the encoded CD8 co-receptor. In specific embodiments, a modified immune cell comprises a heterologous polynucleotide encoding iCasp9 and a heterologous polynucleotide encoding a recombinant CD8 co-receptor protein that comprises a β chain containing a RQR polypeptide and further comprises a CD8 α-chain.

In further embodiments, a host cell comprises a heterologous polynucleotide encoding iCasp9 and a heterologous polynucleotide encoding a recombinant CD8 co-receptor protein that comprises an α-chain containing a RQR polypeptide and further comprises a CD8 β-chain. In some embodiments, both of the encoded CD8 α-chain and the encoded CD8 β-chain contain a RQR polypeptide.

A host cell may be efficiently transduced to contain, and may efficiently express, a single polynucleotide that encodes the binding protein, safety switch protein, selection marker, and CD8 co-receptor protein. For example, in some embodiments, a host cell of the present disclosure comprises a heterologous polynucleotide that encodes, from 5′ to 3′, ([an iCasp9 polypeptide]-[a porcine teschovirus 2A (P2A) peptide]-[a TCR β chain]-[a P2A peptide]-[a TCR α chain]-[a P2A peptide]-[a CD8 (3-chain comprising an RQR polypeptide]-[a P2A peptide]-[a CD8 α-chain]).

In some embodiments, a polynucleotide encoding a binding protein (or a host cell comprising such a polynucleotide) can further comprise: (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii). Without being bound by theory, in certain embodiments, co-expression or concurrent expression of a binding protein and a CD8 co-receptor protein or portion thereof functional to bind to an HLA molecule may improve one or more desired activity of a host cell (e.g., immune cell, such as a T cell, optionally a CD4⁺ T cell) as compared to expression of the binding protein alone. It will be understood that the binding protein-encoding polynucleotide and the CD8 co-receptor polypeptide-encoding polynucleotide may be present on a single nucleic acid molecule (e.g., in a same expression vector), or may be present on separate nucleic acid molecules in a host cell.

In certain further embodiments, a polynucleotide comprises (or a host cell comprises a polynucleotide comprising): (a) the polynucleotide encoding a polypeptide comprising an extracellular portion of a CD8 co-receptor α chain; (b) the polynucleotide encoding a polypeptide comprising an extracellular portion of a CD8 co-receptor β chain; and (c) a polynucleotide encoding a self-cleaving peptide disposed between the polynucleotide of (a) and the polynucleotide of (b). In further embodiments, a polynucleotide comprises a polynucleotide that encodes a self-cleaving peptide and is disposed between: (1) the polynucleotide encoding a binding protein (e.g., TCR of the present disclosure) and the polynucleotide encoding a polypeptide comprising an extracellular portion of a CD8 co-receptor α chain; and/or (2) the polynucleotide encoding a binding protein and the polynucleotide encoding a polypeptide comprising an extracellular portion of a CD8 co-receptor β chain.

In still further embodiments, a polynucleotide can comprise, operably linked in-frame: (i) (pnCD8α)-(pnSCP1)-(pnCD8β)-(pnSCP2)-(pnTCR); (ii) (pnCD8β)-(pnSCP1)-(pnCD8α)-(pnSCP2)-(pnTCR); (iii) (pnTCR)-(pnSCP1)-(pnCD8α)-(pnSCP2)-(pnCD8β); (iv) (pnTCR)-(pnSCP1)-(pnCD8β)-(pnSCP2)-(pnCD8α); (v) (pnCD8α)-(pnSCP1)-(pnTCR)-(pnSCP2)-(pnCD8β); or (vi) (pnCD8β)-(pnSCP1)-(pnTCR)-(pnSCP2)-(pnCD8α), wherein pnCD8a is the polynucleotide encoding a polypeptide that comprises an extracellular portion of a CD8 co-receptor α chain, wherein pnCD8β is the polynucleotide encoding a polypeptide that comprises an extracellular portion of a CD8 co-receptor α chain, wherein pnTCR is the polynucleotide encoding a TCR, and wherein pnSCP1 and pnSCP2 are each independently a polynucleotide encoding a self-cleaving peptide, wherein the polynucleotides and/or the encoded self-cleaving peptides are optionally the same or different (e.g., P2A, T2A, F2A, E2A).

In some embodiments, the encoded TCR comprises a TCRα chain and a TCRβ chain, wherein the polynucleotide comprises a polynucleotide encoding a self-cleaving peptide disposed between the polynucleotide encoding a TCRα chain and the polynucleotide encoding a TCRβ chain. In some embodiments, the polynucleotide comprises, operably linked in-frame: (i) (pnCD8α)-(pnSCP₁)-(pnCD8β)-(pnSCP₂)-(pnTCRβ)-(pnSCP₃)-(pnTCRα); (ii) (pnCD8β)-(pnSCP₁)-(pnCD8α)-(pnSCP₂)-(pnTCRβ)-(pnSCP₃)-(pnTCRα); (iii) (pnCD8α)-(pnSCP₁)-(pnCD8β)-(pnSCP₂)-(pnTCRα)-(pnSCP₃)-(pnTCRβ); (iv) (pnCD8β)-(pnSCP₁)-(pnCD8α)-(pnSCP₂)-(pnTCRα)-(pnSCP₃)-(pnTCRβ); (v) (pnTCR)-(pnSCP₁)-(pnTCRα)-(pnSCP₂)-(pnCD8α)-(pnSCP₃)-(pnCD8β); (vi) (pnTCR)-(pnSCP₁)-(pnTCRα)-(pnSCP₂)-(pnCD8β)-(pnSCP₃)-(pnCD8α); (vii) (pnTCRα)-(pnSCP₁)-(pnTCRβ)-(pnSCP₂)-(pnCD8α)-(pnSCP₃)-(pnCD8β); or (viii) (pnTCRα)-(pnSCP₁)-(pnTCRβ)-(pnSCP₂)-(pnCD8β)-(pnSCP₃)-(pnCD8α), wherein pnCD8α is the polynucleotide encoding a polypeptide that comprises an extracellular portion of a CD8 co-receptor α chain, wherein pnCD8β is the polynucleotide encoding a polypeptide that comprises an extracellular portion of a CD8 co-receptor α chain, wherein pnTCRα is the polynucleotide encoding a TCR α chain, wherein pnTCRβ is the polynucleotide encoding a TCR β chain, and wherein pnSCP₁, pnSCP₂, and pnSCP₃ are each independently a polynucleotide encoding a self-cleaving peptide, wherein the polynucleotides and/or the encoded self-cleaving peptides are optionally the same or different.

In certain embodiments, at least a portion of the isolated polynucleotide is codon-optimized for expression in a host cell (e.g., an antigen-presenting cell or engineered immune cell as disclosed herein).

In particular, any of the aforementioned heterologous polynucleotides comprised in the host cells (e.g., encoding any of the binding proteins of the present disclosure) may also or alternatively be provided in an isolated form. In some embodiments, the polynucleotide is codon-optimized for expression in a host cell.

In certain embodiments, a heterologous polynucleotide encoding a TCR Vα or α-polypeptide and a heterologous polynucleotide encoding a TCR Vβ or β-polypeptide are contained in a single open reading frame comprised in the host cell, wherein the single open reading frame further comprises a polynucleotide encoding a self-cleaving peptide disposed between the Vα (or a polypeptide)-encoding polynucleotide and the Vβ (or β-polypeptide)-encoding polynucleotide.

In any of the embodiments described herein, an isolated polynucleotide is codon-optimized for expression in an immune cell, such as a T cell.

Also provided herein are vectors that comprise a transgene construct of the instant disclosure. Some examples of vectors include plasmids, viral vectors, cosmids, and others. Some vectors may be capable of autonomous replication in a host cell into which they are introduced (e.g., bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding binding proteins as described herein) are co administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to affect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomega¬lovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentivirus” refers to a genus of the retroviridae family. “Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be or be derived from a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing TCR or CAR transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

In certain embodiments, a vector is capable of delivering the transgene construct to a host cell (e.g., a hematopoietic progenitor cell or a human immune system cell). In specific embodiments, a vector is capable of delivering a transgene construct to human immune system cell, such as, for example, a CD4⁺ T cell, a CD8⁺ T cell, a CD4⁻CD8 ⁻ double negative T cell, a γδ T cell, a natural killer cell, a dendritic cell, or any combination thereof. In further embodiments, a vector is capable of delivering a transgene construct to a naïve T cell, a central memory T cell, an effector memory T cell, or any combination thereof. In some embodiments, a vector that encodes a polynucleotide or transgene construct of the present disclosure may further comprise a polynucleotide that encodes a nuclease that can be used to perform a chromosomal knockout in a host cell (e.g., a CRISPR-Cas endonuclease or another endonuclease as disclosed herein) or that can be used to to deliver a therapeutic transgene or portion thereof to a host cell in a gene therapy replacement or gene repair therapy. Alternatively, a nuclease used for a chromosomal knockout or a gene replacement or gene repair therapy can be delivered to a host cell independent of a vector that encodes a polynucleotide or transgene construct of this disclosure.

### Uses

In additional aspects, the present disclosure provides methods of eliciting an immune response (e.g., cytokine production, proliferation of T cells, activation of T cells (e.g., increased expression of CD137, production of intracellular calcium, increased phosphorylation of a TCR signaling protein), proliferation of B cells, production of antibodies, or any combination thereof) against a cancer associated with expression of a SOX2 antigen according to any one of SEQ ID NOs:2-7, such as a hematological malignancy (e.g., multiple myeloma), wherein the methods comprise administering to a human subject having or suspected of having the cancer an effective amount of an immunogenic composition or host cell expressing the same, a SOX2-specific binding protein, a SOX2-specific fusion protein, or a host cell, such as a T cell, that encodes and is capable of expressing a SOX2-specific binding protein as disclosed herein.

Also provided are methods for expanding a population of T cells that specifically bind to a SOX2 antigen according to any one of SEQ ID NOs:2-7, optionally in the context of an antigen:HLA complex, wherein the methods comprise contacting a sample comprising one or more T cells that specifically bind to the antigen with an immunogenic composition (or host cell encoding an immunogenic SOX2 peptide or fusion polypeptide) of the present disclosure.

Also provided are methods for generating and/or isolating T cells, wherein the methods comprise contacting peripheral blood cells or whole blood with: (a) an immunogenic composition of the present disclosure; or (b) antigen-presenting cells (APCs) that express (e.g., present in the context of an HLA molecule) or have been pulsed with a SOX2 antigen comprising an amino acid sequence according to any one or more of SEQ ID NOs:2-7, or (iv) any combination of (i)-(iii); and optionally sorting T cells from the peripheral blood cells, thereby isolating and/or generating T cells

In still other aspects, the present disclosure provides methods for treating a disease or disorder associated with (e.g. that expresses or is believed or known to express or is confirmed to express) a SOX2 antigen according to any one of SEQ ID NOs:2-7 in a subject, wherein the methods comprise administering to the subject a SOX2-specific host (e.g. immune) cell, binding protein, polynucleotide, vector, fusion protein, composition, or immunogenic composition of the present disclosure, thereby treating the disease or condition. Also provided are presently disclosed binding proteins, polynucleotides, vectors, host cells, compositions, and immunogenic compositions for use in the treatment of a disease or disorder associated with a SOX2 antigen according to any one of SEQ ID NOs:2-7 and/or for use in the manufacture of a medicament for treating a disease or disorder associated with a SOX2 antigen according to any one of SEQ ID NOs:2-7.

The terms “treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a modified immune cell of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount”, as used herein, refers to an amount of a composition sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient.

As used herein, “statistically significant” refers to a p value of 0.050 or less when calculated using the Students t-test and indicates that it is unlikely that a particular event or result being measured has arisen by chance.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

In certain embodiments, a subject treated according to the presently disclosed methods is HLA-A*02:01*. In some embodiments, the disease or condition is a cancer. In certain embodiments, the cancer comprises a hematological malignancy or a solid tumor.

In certain embodiments, the hematological malignancy comprises a myeloma, such as, for example, multiple myeloma. In certain embodiments, the hematological malignancy comprises a leukemia (e.g., an acute leukemia or a chronic leukemia). In specific embodiments, the leukemia comprises acute myeloid leukemia (AML), acute lymphocytic leukemia (ALL), mixed phenotype acute leukemia (MPAL), chronic myeloid leukemia (CML), B cell prolymphocytic leukemia, hairy cell leukemia, or chronic lymphocytic leukemia (CLL). In certain embodiments, the hematological malignancy comprises a lymphoma. In certain embodiments, the lymphoma comprises Hodgkin's lymphoma (HL), non-Hodgkin's lymphoma (NHL), a central nervous system lymphoma, small lymphocytic lymphoma (SLL), CD37+ dendritic cell lymphoma, lymphoplasmacytic lymphoma, splenic marginal zone lymphoma, plasma cell myeloma, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, precursor B-lymphoblastic lymphoma, immunoblastic large cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder. In certain embodiments, the hematological malignancy comprises a myelodysplastic disorder, such as, for example, refractory cytopenia with unilineage dysplasia (refractory anemia, refractory neutropenia, and refractory thrombocytopenia), refractory anemia with ring sideroblasts (RARS), refractory anemia with ring sideroblasts-thrombocytosis (RARS-t), refractory cytopenia with multinieage dysplasia (RCMD), refractory cytopenia with multinieage dysplasia and ring sideroblasts (RCMD-RS), refractory anemia with excess blasts (RAEB), myelodysplasia unclassifiable, and refractory cytopenia of childhood.

Exemplary cancers that can form solid tumors that can be targeted or treated with the compositions and methods of this disclosure include sarcomas and carcinomas, including, for example, chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; synovial sarcoma; Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); a lung carcinoma (e.g., Adenocarcinoma, Squamous Cell Carcinoma (Epidermoid Carcinoma), Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma), neuroblastoma, hepatoblastoma, Brain tumors subtypes (e.g., gliomas, PNETs, cranipharyngioma, choroid plexus tumors, schwannomas, meningiomas), Wilms tumor, Germ cell tumors.

In certain embodiments, methods of the present disclosure target or treat a solid tumor formed by a cancer selected from an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In some embodiments, a cancer comprises multiple myeloma, plasma cell leukemia, ovarian cancer, glioma (see e.g. Schmitz et al. Br J Cancer 96(8):1293-1301 (2007)), lung cancer, neck cancer, cervical cancer, or any combination thereof.

The level of an immune response against a solid tumor (e.g., a CTL (cytotoxic T lymphocyte) immune response) may be determined by any one of numerous immunological methods described herein. The level of a CTL immune response may be determined prior to and following administration of any one of the herein described antigen-specific binding receptors expressed by, for example, a T cell. Cytotoxicity assays for determining CTL activity may be performed using any one of several techniques and methods (see, e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein); IncuCyte® assays (e.g., as described herein).

In some embodiments, a cancer treatable by a method of the present disclosure comprises glioblastoma, medulloblastoma, breast cancer, colorectal cancer, lung cancer (e.g., non-small cell lung cancer), esophageal cancer, lymphoma, leukemia (e.g., acute myeloid leukemia), melanoma, cholangiocarcinoma, bladder cancer, cervical cancer, pancreatic cancer, or hepatocellular carcinoma, neuroblastoma, hepatoblastoma, Brain tumors subtypes (e.g., gliomas, PNETs, cranipharyngioma, choroid plexus tumors, schwannomas, meningiomas), Wilms tumor, Germ cell tumors.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. A pediatric subject refers to an infant, juvenile, or adolescent subject. Cells according to the present disclosure may be administered in a manner appropriate to the disease, condition, or disorder to be treated as determined by persons skilled in the medical art. In any of the above embodiments, an engineered immune cell or unit dose as described herein is administered intravenously, intraperitoneally, intratumorally, intracerebrally, into the bone marrow, into a lymph node, or into the cerebrospinal fluid so as to encounter target cells (e.g., cancer cells). An appropriate dose, suitable duration, and frequency of administration of the compositions will be determined by such factors as a condition of the patient; size, type, and severity of the disease, condition, or disorder; the particular form of the active ingredient; and the method of administration.

For therapeutic host cells, the amount of cells in a composition or unit dose is at least one cell (for example, one SOX2-specific CD8⁺ T cell subpopulation; one SOX2-specific CD4⁺ T cell subpopulation) or is more typically greater than 10² cells, for example, up to 10⁶, up to 10⁷, up to 10⁸ cells, up to 10⁹ cells, or more than 10¹⁰ cells, such as 10¹¹ cells. In certain embodiments, the cells are administered in a range from about 10⁶ to about 10¹⁰ cells/m², preferably in a range of about 10⁵ to about 10¹¹ cells/m². The number of cells will depend upon the ultimate use for which the composition is intended as well the type of cells included therein. For example, cells modified to contain a fusion protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 104 cells/ml and generally is greater than 107 cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells. In certain embodiments, a unit dose of the modified immune cells can be co-administered with (e.g., simultaneously or contemporaneously) hematopoietic stem cells from an allogeneic donor.

Also contemplated are pharmaceutical compositions (i.e., compositions) comprising a SOX2-specific binding protein, polynucleotide, vector, host cell, fusion protein, or immunogenic composition as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

Generally, a therapeutically effective dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In some embodiments, a vector is provided that comprises a DNA plasmid construct encoding a TCR-mimic antibody or antigen-binding fragment, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

The pharmaceutical compositions described herein may be presented in unit-dose or multi-dose containers, such as sealed ampoules or vials. Such containers may be frozen to preserve the stability of the formulation until infusion into the patient. In certain embodiments, a unit dose comprises a SOX2-specific immune cell as described herein at a dose of about 10⁷ cells/m² to about 10¹¹ cells/m². The development of suitable dosing and treatment regimens for using the particular compositions described herein in a variety of treatment regimens, including, e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In general, an appropriate dosage and treatment regimen provides the active molecules or cells in an amount sufficient to provide a benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., engineered immune cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a binding protein, polynucleotide, vector, host cell, or composition described herein is administered to the subject, which may be administered at intervals between administrations of about two to about four weeks.

Treatment or prevention methods of this disclosure may be administered to a subject as part of a treatment course or regimen, which may comprise additional treatments prior to, or after, administration of the instantly disclosed unit doses, cells, or compositions. For example, in certain embodiments, a subject receiving a unit dose of the host cell is receiving or had previously received a hematopoietic cell transplant (HCT; including myeloablative and non-myeloablative HCT). Techniques and regimens for performing HCT are known in the art and can comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in certain embodiments, a host cell of the present disclosure can be administered with or shortly after hematopoietic stem cells in a modified HCT therapy.

In further embodiments, the subject had previously received lymphodepleting chemotherapy prior to receiving the SOX2-specific immune cells or HCT. In certain embodiments, a lymphodepleting chemotherapy comprises a conditioning regimen comprising cyclophosphamide, fludarabine, anti-thymocyte globulin, or a combination thereof.

In certain embodiments, the subject had previously received one or more of surgery; radiation therapy, or chemotherapy, which therapies include those described herein or otherwise known in the art. In certain embodiments, for example, chemotherapy comprises vincristine, cisplatin, cyclophosphamide, filgrastim, etoposide, thiotepa, or any combination thereof.

Methods according to this disclosure may further include administering one or more additional agents to treat the disease or disorder in a combination therapy. For example, in certain embodiments, a combination therapy comprises administering a presently disclosed composition (e.g. binding protein, polynucleotide, vector, host cell, or composition) with (concurrently, simultaneously, or sequentially) an immune checkpoint inhibitor. In some embodiments, a combination therapy comprises administering a composition (e.g. binding protein, polynucleotide, vector, host cell, or composition) with an agonist of a stimulatory immune checkpoint agent. In further embodiments, a combination therapy comprises administering a composition (e.g. binding protein, polynucleotide, vector, host cell, or composition) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVR1G (CD112R), PVRL2, adenosine, A2aR, immunosuppressive cytokines (e.g., IL-10, IL-4, IL-iRA, IL-35), IDO, arginase, VISTA, TIGIT, LAIR1, CEACAM-1, CEACAM-3, CEACAM-5, Treg cells, or any combination thereof. An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule.

In any of the embodiments disclosed herein, a method may comprise a composition (e.g. binding protein, polynucleotide, vector, host cell, or composition) with one or more inhibitor of any one of the following immune suppression components, singly or in any combination. In certain embodiments, a composition (e.g. binding protein, polynucleotide, vector, host cell, or composition) is used in combination with a PD-1 inhibitor, for example a PD-1-specific antibody or binding fragment thereof, such as pidilizumab, nivolumab, pembrolizumab, MEDI0680 (formerly AMP-514), AMP-224, BMS-936558 or any combination thereof. In further embodiments, a composition (e.g. binding protein, polynucleotide, vector, host cell, or composition) of the present disclosure is used in combination with a PD-L1 specific antibody or binding fragment thereof, such as BMS-936559, durvalumab (EDI4736), atezolizumab (RG7446), avelumab (MSB0010718C), MPDL3280A, or any combination thereof. In certain embodiments, a composition (e.g. binding protein, polynucleotide, vector, host cell, or composition) of the present disclosure is used in combination with a LAG3 inhibitor, such as LAG525, IMP321, IMP701, 9H12, BMS-986016, or any combination thereof. In certain embodiments, a composition (e.g. binding protein, polynucleotide, vector, host cell, or composition) is used in combination with an inhibitor of CTLA4. In particular embodiments, a SOX2-specific immune cell is used in combination with a CTLA4 specific antibody or binding fragment thereof, such as ipilimumab, tremelimumab, CTLA4-Ig fusion proteins (e.g., abatacept, belatacept), or any combination thereof. In certain embodiments, a composition (e.g. binding protein, polynucleotide, vector, host cell, or composition) is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1. In certain embodiments, a composition (e.g. binding protein, polynucleotide, vector, host cell, or composition) is used in combination with an inhibitor of CD244. In certain embodiments, a composition (e.g. binding protein, polynucleotide, vector, host cell, or composition) is used in combination with an inhibitor of BLTA, HVEM, CD160, or any combination thereof. Anti CD-160 antibodies are described in, for example, PCT Publication No. WO 2010/084158. In certain embodiments, a composition (e.g. binding protein, polynucleotide, vector, host cell, or composition) is used in combination with an inhibitor of TIM3. In certain embodiments, a composition (e.g. binding protein, polynucleotide, vector, host cell, or composition) is used in combination with an inhibitor of Gal9. In certain embodiments, a composition (e.g. binding protein, polynucleotide, vector, host cell, or composition) is used in combination with an inhibitor of adenosine signaling, such as a decoy adenosine receptor. In certain embodiments, a composition (e.g. binding protein, polynucleotide, vector, host cell, or composition) is used in combination with an inhibitor of A2aR. In certain embodiments, a composition (e.g. binding protein, polynucleotide, vector, host cell, or composition) is used in combination with an inhibitor of KIR, such as lirilumab (BMS-986015). In certain embodiments, a composition (e.g. binding protein, polynucleotide, vector, host cell, or composition) is used in combination with an inhibitor of an inhibitory cytokine (typically, a cytokine other than TGFβ) or Treg development or activity. In certain embodiments, a composition (e.g. binding protein, polynucleotide, vector, host cell, or composition) is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof. In certain embodiments, a composition (e.g. binding protein, polynucleotide, vector, host cell, or composition) is used in combination with an arginase inhibitor, such as N(omega)-Nitro-L-arginine methyl ester (L-NAME), N-omega-hydroxy-nor-l-arginine (nor-NOHA), L-NOHA, 2(S)-amino-6-boronohexanoic acid (ABH), S-(2-boronoethyl)-L-cysteine (BEC), or any combination thereof. In certain embodiments, a composition (e.g. binding protein, polynucleotide, vector, host cell, or composition) is used in combination with an inhibitor of VISTA, such as CA-170 (Curis, Lexington, Mass.). In certain embodiments, a composition (e.g. binding protein, polynucleotide, vector, host cell, or composition) is used in combination with an inhibitor of TIGIT such as, for example, COM902 (Compugen, Toronto, Ontario Canada), an inhibitor of CD155, such as, for example, COM701 (Compugen), or both. In certain embodiments, a composition (e.g. binding protein, polynucleotide, vector, host cell, or composition) is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526. In certain embodiments, a composition (e.g. binding protein, polynucleotide, vector, host cell, or composition) is used in combination with a LAIR1 inhibitor. In certain embodiments, a composition (e.g. binding protein, polynucleotide, vector, host cell, or composition) is used in combination with an inhibitor of CEACAM-1, CEACAM-3, CEACAM-5, or any combination thereof.

In certain embodiments, a composition (e.g. binding protein, polynucleotide, vector, host cell, or composition) used in combination with an agent that increases the activity (i.e., is an agonist) of a stimulatory immune checkpoint molecule. For example a composition can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized anti-GITR monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a composition (e.g. binding protein, polynucleotide, vector, host cell, or composition) with one or more agonist of a stimulatory immune checkpoint molecule, including any of the foregoing, singly or in any combination.

In certain embodiments, a combination therapy comprises a composition (e.g. binding protein, polynucleotide, vector, host cell, or composition) and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy comprises administering a composition (e.g. binding protein, polynucleotide, vector, host cell, or composition) and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer in a subject are well-known to those of ordinary skill in the art.

In certain embodiments, a combination therapy method comprises administering a composition (e.g. binding protein, polynucleotide, vector, host cell, or composition) and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates—busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with a composition (e.g. binding protein, polynucleotide, vector, host cell, or composition) of this disclosure.

The present disclosure also provides the following non-limiting Embodiments:

Embodiment 1. An isolated binding protein that is capable of binding to a SOX2 peptide antigen:HLA complex, wherein the SOX2 peptide antigen comprises or consists of the amino acid sequence set forth in SEQ ID NO.:5, 2, 3, 4, 6, or 7, and wherein, optionally, the binding comprises specific binding.

Embodiment 2. The binding protein of Embodiment 1, wherein the SOX2 peptide antigen comprises or consists of the amino acid sequence set forth in SEQ ID NO.:5.

Embodiment 3. The binding protein of Embodiment 1, wherein the SOX2 peptide antigen comprises or consists of the amino acid sequence set forth in SEQ ID NO.:2.

Embodiment 4. The binding protein of Embodiment 1, wherein the SOX2 peptide antigen comprises or consists of the amino acid sequence set forth in SEQ ID NO.:3.

Embodiment 5. The binding protein of Embodiment 1, wherein the SOX2 peptide antigen comprises or consists of the amino acid sequence set forth in SEQ ID NO.:4.

Embodiment 6. The binding protein of Embodiment 1, wherein the SOX2 peptide antigen comprises or consists of the amino acid sequence set forth in SEQ ID NO.:6.

Embodiment 7. The binding protein of Embodiment 1, wherein the SOX2 peptide antigen comprises or consists of the amino acid sequence set forth in SEQ ID NO.:7.

Embodiment 8. The binding protein of any one of Embodiments 1-7, wherein the HLA comprises HLA-A*02:01.

Embodiment 9. The binding protein of any one of Embodiments 1-8, comprising an immunoglobulin superfamily variable domain.

Embodiment 10. The binding protein of any one of Embodiments 1-9, comprising a TCR α-chain variable domain (Vα) and/or a TCR j-chain variable domain (VR).

Embodiment 11. The binding protein of any one of Embodiments 1-9, comprising a heavy chain variable domain (VH) and/or a light chain variable domain (VL) of a TCR-mimic antibody.

Embodiment 12. The binding protein of any one of Embodiments 1-11, wherein the binding protein comprises: (i) the amino acid sequence set forth in any one of SEQ ID NOs.:52, 53, 100, 101, 16, 17, 28, 29, 40, 41, 64, 65, 76, 77, 88, 89, 112, 113, 124, 125, 136, 137, 148, and 149, or a variant thereof comprising one, two, or three amino acid substitutions, wherein the amino acid sequence is optionally a CDR3β; (ii) the amino acid sequence set forth in any one of SEQ ID NOs.: 51, 99, 15, 27, 39, 63, 75, 87, 111, 123, 135, and 147, or a variant thereof comprising one, two, or three amino acid substitutions, wherein the amino acid sequence is optionally a CDR2β; (iii) the amino acid sequence set forth in any one of SEQ ID NOs.: 50, 98, 14, 26, 38, 62, 74, 86, 110, 122, 134, and 146, or a variant thereof comprising one, two, or three amino acid substitutions, wherein the amino acid sequence is optionally a CDR1β; (iv) the amino acid sequence set forth in any one of SEQ ID NOs.: 57, 58, 105, 106, 21, 22, 33, 34, 45, 46, 69, 70, 81, 82, 93, 94, 117, 118, 129, 130, 141, 142, 153, and 154, or a variant thereof comprising one, two, or three amino acid substitutions, wherein the amino acid sequence is optionally a CDR3α; (v) the amino acid sequence set forth in any one of SEQ ID NOs.: 56, 104, 20, 32, 44, 68, 80, 92, 116, 128, 140, and 152, or a variant thereof comprising one, two, or three amino acid substitutions, wherein the amino acid sequence is optionally a CDR2α; and/or (vi) the amino acid sequence set forth in any one of SEQ ID NOs.: 55, 103, 19, 31, 43, 67, 79, 91, 115, 127, 139, and 151, or a variant thereof comprising one, two, or three amino acid substitutions, wherein the amino acid sequence is optionally a CDR1α.

Embodiment 13. The binding protein of any one of Embodiments 1-10 and 12, comprising: (1) a TCR Vα comprising CDR1α, CDR2α, and CDR3α; and (2) a TCR Vβ comprising CDR1β, CDR2β, and CDR3β, wherein the CDR1α, CDR2α, CDR3α, CDR1β, CDR2β, and CDR3β are as set forth in: (i) SEQ ID NOs.:55, 56, 57 or 58, 50, 51, and 52 or 53, respectively; (ii) SEQ ID NOs.:103, 104, 105 or 106, 98, 99, and 100 or 101, respectively; (iii) SEQ ID NOs.:19, 20, 21 or 22, 14, 15, and 16 or 17, respectively; (iv) SEQ ID NOs.:31, 32, 33 or 34, 26, 27, and 28 or 29, respectively; (v) SEQ ID NOs.:43, 44, 45 or 46, 38, 39, and 40 or 41, respectively; (vi) SEQ ID NOs.:67, 68, 69 or 70, 62, 63, and 64 or 65, respectively; (vii) SEQ ID NOs.:79, 80, 81 or 82, 74, 75, and 76 or 77, respectively; (viii) SEQ ID NOs.:91, 92, 93 or 94, 86, 87, and 88 or 89, respectively; (ix) SEQ ID NOs.:115, 116, 117 or 118, 110, 111, and 112 or 113, respectively; (x) SEQ ID NOs.:127, 128, 129 or 130, 122, 123, and 124 or 125, respectively; (xi) SEQ ID NOs.:139, 140, 141 or 142, 134, 135, and 136 or 137 respectively; or (xii) SEQ ID NOs.:151, 152, 153 or 154, 146, 147, and 148 or 149, respectively.

Embodiment 14. The binding protein of any one of Embodiments 10 and 12-13, wherein the Vα comprises or consists of an amino acid sequence having at least 80%, at least 85%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.: 54, 102, 18, 30, 42, 66, 78, 90, 114, 126, 138, and 150.

Embodiment 15. The binding protein of any one of Embodiments 10 and 12-14 wherein the Vβ comprises or consists of the amino acid sequence having at least 80%, at least 85%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.:49, 97, 13, 25, 37, 61, 73, 85, 109, 121, 133, and 145.

Embodiment 16. The binding protein of any one of Embodiments 10 and 12-15, comprising a Vα and a Vβ that comprise or consist of amino acid sequences having at least 80%, at least 85%, at least 90%, at least 91%, at least 92%, at least 93%, at least 94%, at least 95%, at least 96%, at least 97%, at least 98%, or at least 99% identity to the amino acid sequences set forth in: (i) SEQ ID NOs.:54 and 49, respectively; (ii) SEQ ID NOs.:102 and 97, respectively; (iii) SEQ ID NOs.:18 and 13, respectively; (iv) SEQ ID NOs.:30 and 25, respectively; (v) SEQ ID NOs.:42 and 37, respectively; (vi) SEQ ID NOs.:66 and 61, respectively; (vii) SEQ ID NOs.:78 and 73, respectively; (viii) SEQ ID NOs.:90 and 85, respectively; (ix) SEQ ID NOs.:114 and 109, respectively; (x) SEQ ID NOs.:126 and 121, respectively; (xi) SEQ ID NOs.:138 and 133 respectively; or

(xii) SEQ ID NOs.:150 and 145, respectively.

Embodiment 17. The binding protein of any one of Embodiments 10 and 12-16, comprising a Vα and a Vβ that comprise or consist of the amino acid sequences according to: (i) SEQ ID NOs.:54 and 49, respectively; (ii) SEQ ID NOs.:102 and 97, respectively; (iii) SEQ ID NOs.:18 and 13, respectively; (iv) SEQ ID NOs.:30 and 25, respectively; (v) SEQ ID NOs.:42 and 37, respectively; (vi) SEQ ID NOs.:66 and 61, respectively; (vii) SEQ ID NOs.:78 and 73, respectively; (viii) SEQ ID NOs.:90 and 85, respectively; (ix) SEQ ID NOs.:114 and 109, respectively; (x) SEQ ID NOs.:126 and 121, respectively; (xi) SEQ ID NOs.:138 and 133 respectively; or (xii) SEQ ID NOs.:150 and 145, respectively.

Embodiment 18. The binding protein of any one of Embodiments 10 and 12-17, wherein the binding protein is comprised in an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:48, 96, 12, 24, 36, 60, 72, 84, 108, 120, 132, and 144.

Embodiment 19. The binding protein of any one of Embodiments 1-11, wherein the binding protein comprises: (i) a CDR3β, a CDR2β, and/or a CDR1β of a TCR of a T cell from: Donor 1 Line 1, Donor 1 Line 3, Donor 2 Line 8, Donor 2 Line 1, Donor 2 Line 13, Donor 2 Line 11, or Donor 2 Line 19; and/or (ii) a CDR3α, a CDR2α, and/or a CDR1α of a TCR of a T cell from: Donor 1 Line 1, Donor 1 Line 3, Donor 2 Line 8, Donor 2 Line 1, Donor 2 Line 13, Donor 2 Line 11, or Donor 2 Line 19.

Embodiment 20. The binding protein of any one of Embodiments 1-11, wherein the binding protein comprises: (i) a Vα having at least 90% amino acid identity to the Vα of a TCR of a T cell from: Donor 1 Line 1, Donor 1 Line 3, Donor 2 Line 8, Donor 2 Line 1, Donor 2 Line 13, Donor 2 Line 11, or Donor 2 Line 19; and/or (ii) a Vβ having at least 90% amino acid identity to the Vβ of a TCR of a T cell from: Donor 1 Line 1, Donor 1 Line 3, Donor 2 Line 8, Donor 2 Line 1, Donor 2 Line 13, Donor 2 Line 11, or Donor 2 Line 19, provided that (a) at least three or four of the CDRs have no mutations; (b) the CDRs that do have mutations have only up to two amino acid substitutions, up to a contiguous five amino acid deletion, or a combination thereof; and

(c) the binding protein retains its ability to bind to a SOX2 peptide:HLA complex.

Embodiment 21. The binding protein of any one of Embodiments 1-20, further comprising a TCR β-polypeptide constant domain (Cβ), a TCR α-polypeptide constant domain (Cα), or both, wherein, optionally, (1) the Vβ and the Cβ together comprise a TCR β chain and/or the Vα and the Cα together comprise a TCR α chain, and/or (2) the Cβ comprises or consists of an amino acid sequence having at least at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NO.:156 or 157, and/or the Cα comprises or consists of an amino acid sequence having at least at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NO.:155.

Embodiment 22. The binding protein of Embodiment 21, comprising a TCR Cβ and a TCR Cα, wherein the Cβ and/or the Cα comprises one or more non-native amino acid at a position such that when the the Cβ and the Cα associate to form a dimer, a non-native disulfide bond is formed between the Cβ and the Cα, wherein, optionally, the non-native amino acid comprises a cysteine in the Cβ and/or a cysteine in the Cα.

Embodiment 23. The binding protein of Embodiment 21 ir 22, comprising a TCR Cβ and a TCR Cα, wherein the TCR Cβ comprises a cysteine amino acid at amino acid position 57, and wherein the TCR Cα comprises a cysteine amino acid at amino acid position 48.

Embodiment 24. The binding protein of any one of Embodiments 1-23, wherein the binding protein comprises a TCR, a single-chain TCR (scTCR), a scTv, a chimeric antigen receptor (CAR), a TCR-mimic antibody or an antigen-binding fragment thereof, or any combination thereof.

Embodiment 25. The binding protein of Embodiment 24, wherein binding protein comprises a TCR.

Embodiment 26. The binding protein of Embodiment 24, wherein the binding protein comprises a scTv.

Embodiment 27. The binding protein of Embodiment 24, wherein the binding protein comprises a scTCR.

Embodiment 28. The binding protein of Embodiment 24, wherein the binding protein comprises a CAR.

Embodiment 29. The binding protein of Embodiment 24, wherein the binding protein comprises a TCR-mimic antibody or an antigen-binding fragment thereof.

Embodiment 30. An isolated polynucleotide encoding the binding protein of any one of Embodiments 1-29.

Embodiment 31. The polynucleotide of Embodiment 30, wherein the polynucleotide is codon-optimized for expression in a host cell, wherein, optionally, the host cell comprises an immune system cell, wherein, further optionally, the immune system cell comprises a T cell, a NK-T cell, or a NK cell.

Embodiment 32. The polynucleotide of Embodiment 31, further comprising:
(i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

Embodiment 33. The polynucleotide of Embodiment 32, comprising: (a) the polynucleotide encoding a polypeptide comprising an extracellular portion of a CD8 co-receptor α chain; (b) the polynucleotide encoding a polypeptide comprising an extracellular portion of a CD8 co-receptor β chain; and (c) a polynucleotide encoding a self-cleaving peptide disposed between the polynucleotide of (a) and the polynucleotide of (b).

Embodiment 34. The polynucleotide of Embodiment 32 or 33, further comprising a polynucleotide that encodes a self-cleaving peptide and is disposed between: (1) the polynucleotide encoding a binding protein and the polynucleotide encoding a polypeptide comprising an extracellular portion of a CD8 co-receptor α chain; and/or

(2) the polynucleotide encoding a binding protein and the polynucleotide encoding a polypeptide comprising an extracellular portion of a CD8 co-receptor β chain.

Embodiment 35. The polynucleotide of any one of Embodiments 32-34, comprising, operably linked in-frame: (i) (pnCD8α)-(pnSCP₁)-(pnCD8β)-(pnSCP₂)-(pnBP); (ii) (pnCD8β)-(pnSCP₁)-(pnCD8α)-(pnSCP₂)-(pnBP); (iii) (pnBP)-(pnSCP₁)-(pnCD8α)-(pnSCP₂)-(pnCD8β); (iv) (pnBP)-(pnSCP₁)-(pnCD8β)-(pnSCP₂)-(pnCD8α); (v) (pnCD8α)-(pnSCP₁)-(pnBP)-(pnSCP₂)-(pnCD8β); or (vi) (pnCD8β)-(pnSCP₁)-(pnBP)-(pnSCP₂)-(pnCD8α), wherein pnCD8α is the polynucleotide encoding a polypeptide that comprises an extracellular portion of a CD8 co-receptor α chain, wherein pnCD8β is the polynucleotide encoding a polypeptide that comprises an extracellular portion of a CD8 co-receptor α chain, wherein pnBP is the polynucleotide encoding a binding protein, and wherein pnSCP₁ and pnSCP₂ are each independently a polynucleotide encoding a self-cleaving peptide, wherein the polynucleotides and/or the encoded self-cleaving peptides are optionally the same or different.

Embodiment 36. The polynucleotide of any one of Embodiments 30-35, wherein the encoded binding protein comprises a TCRα chain and a TCRβ chain, wherein the polynucleotide comprises a polynucleotide encoding a self-cleaving peptide disposed between the polynucleotide encoding a TCRα chain and the polynucleotide encoding a TCRβ chain.

Embodiment 37. The polynucleotide of Embodiment 36, comprising, operably linked in-frame: (i) (pnCD8α)-(pnSCP₁)-(pnCD8β)-(pnSCP₂)-(pnTCRβ)-(pnSCP₃)-(pnTCRα); (ii) (pnCD8β)-(pnSCP₁)-(pnCD8α)-(pnSCP₂)-(pnTCRβ)-(pnSCP₃)-(pnTCRα);
(iii) (pnCD8α)-(pnSCP₁)-(pnCD8β)-(pnSCP₂)-(pnTCRα)-(pnSCP₃)-(pnTCRO);
(iv) (pnCD8β)-(pnSCP₁)-(pnCD8α)-(pnSCP₂)-(pnTCRα)-(pnSCP₃)-(pnTCRO);
(v) (pnTCRβ)-(pnSCP₁)-(pnTCRα)-(pnSCP₂)-(pnCD8α)-(pnSCP₃)-(pnCD8β);
(vi) (pnTCRβ)-(pnSCP₁)-(pnTCRα)-(pnSCP₂)-(pnCD8β)-(pnSCP₃)-(pnCD8α);
(vii) (pnTCRα)-(pnSCP₁)-(pnTCRβ)-(pnSCP₂)-(pnCD8α)-(pnSCP₃)-(pnCD8β); or
(viii) (pnTCRα)-(pnSCP₁)-(pnTCRβ)-(pnSCP₂)-(pnCD8β)-(pnSCP₃)-(pnCD8α),
wherein pnCD8α is the polynucleotide encoding a polypeptide that comprises an extracellular portion of a CD8 co-receptor α chain, wherein pnCD8β is the polynucleotide encoding a polypeptide that comprises an extracellular portion of a CD8 co-receptor α chain, wherein pnTCRα is the polynucleotide encoding a TCR α chain, wherein pnTCRβ is the polynucleotide encoding a TCR β chain, and wherein pnSCP₁, pnSCP₂, and pnSCP₃ are each independently a polynucleotide encoding a self-cleaving peptide, wherein the polynucleotides and/or the encoded self-cleaving peptides are optionally the same or different.

Embodiment 38. The polynucleotide of any one of Embodiments 30-37, wherein the polynucleotide comprises DNA, RNA (optionally mRNA), or both.

Embodiment 39. The polynucleotide of Embodiment 38, comprising DNA.

Embodiment 40. The polynucleotide of any one of Embodiments 30-39, wherein: (1) the polynucleotide encodes an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:48, 96, 12, 24, 36, 60, 72, 84, 108, 120, 132, and 144; and/or (2) the polynucleotide comprises a polynucleotide having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprises or consists of, the nucleotide acid sequence set forth in any one of SEQ ID NOs.: 47, 95, 11, 23, 35, 47, 59, 71, 83, 107, 119, 131, and 143.

Embodiment 41. A vector comprising the polynucleotide of any one of Embodiments 30-40.

Embodiment 42. The vector of Embodiment 41, wherein the vector comprises a viral vector.

Embodiment 43. The vector of Embodiment 42, wherein the viral vector comprises a lentiviral vector or a 7-retroviral vector.

Embodiment 44. The vector of any one of Embodiments 41-43, wherein the vector is capable of delivering the polynucleotide to a host cell.

Embodiment 45. The vector of Embodiment 44, wherein the host cell is a hematopoietic progenitor cell or a human immune system cell.

Embodiment 46. The vector of Embodiment 45, wherein the human immune system cell is a CD4⁺ T cell, a CD8⁺ T cell, a CD4⁻CD8⁻ double negative T cell, a γδ T cell, a natural killer cell, a natural killer T cell, a macrophage, a monocyte, a dendritic cell, or any combination thereof.

Embodiment 47. The vector of Embodiment 46, wherein the T cell is a naïve T cell, a central memory T cell, an effector memory T cell, or any combination thereof.

Embodiment 48. A host cell comprising the polynucleotide of any one of Embodiments 30-40 and/or the vector of any one of Embodiments 41-47, and/or expressing the binding protein of any one of Embodiments 1-29, wherein the polynucleotide, vector, or binding protein is optionally heterologous to the host cell.

Embodiment 49. The host cell of Embodiment 48, wherein the host cell comprises a hematopoietic progenitor cell and/or an immune cell, optionally a human immune cell.

Embodiment 50. The host cell of Embodiment 49, wherein the host cell comprises a T cell, a NK cell, a NK-T cell, a dendritic cell, a macrophage, a monocyte, a B cell, a plasma cell, or any combination thereof.

Embodiment 51. The host cell of Embodiment 50, wherein the host cell comprises a CD4⁺ T cell, a CD8⁺ T cell, a CD4⁻ CD8⁻ double negative T cell, a γδ T cell, or any combination thereof,
wherein, optionally, the host cell comprises a CD4⁺ T cell and a CD8⁺ T cell, wherein, further optionally, the CD4⁺ T cell, the CD8⁺ T cell, or both comprise (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

Embodiment 52. The host cell of Embodiment 50 or 51, wherein the host cell comprises a CD8⁺ T cell and/or a CD4⁺ T cell.

Embodiment 53. The host cell of any one of Embodiments 48-52, wherein the host cell comprises a chromosomal gene knockout of a PD-1 gene; a LAG3 gene; a TIM3 gene; a CTLA4 gene; an HLA component gene; a TIGIT gene; a TCR component gene, a FasL gene, or any combination thereof.

Embodiment 54. The host cell of Embodiment 53, wherein the chromosomal gene knockout comprises a knockout of an HLA component gene selected from an α1 macroglobulin gene, an α2 macroglobulin gene, an α3 macroglobulin gene, a β1 microglobulin gene, or a β2 microglobulin gene.

Embodiment 55. The host cell of Embodiment 53 or 54, wherein the chromosomal gene knockout comprises a knockout of a TCR component gene selected from a TCR α variable region gene, a TCR β variable region gene, a TCR constant region gene, or a combination thereof.

Embodiment 56. The host cell of any one of Embodiments 48-55, wherein the polynucleotide encoding the binding protein is heterologous to the host cell and is comprised in an endogenous TCR gene locus.

Embodiment 57. The host cell of any one of Embodiments 48-56, further comprising a heterologous polynucleotide encoding: (i) a safety switch protein; (ii) a selection marker; (iii) a CD8 co-receptor β-chain; (iv) a CD8 co-receptor α-chain; or (v) any combination thereof.

Embodiment 58. The host cell of any one of Embodiments 48-57, which produces IFN-γ when in the presence of the SOX2 antigen:HLA complex, wherein, optionally, the SOX2 antigen:HLA complex is expressed on the surface of a target cell.

Embodiment 59. The host cell of any one of Embodiments 48-58, wherein the binding protein is capable of binding to the YLPGAEVPEPA (SEQ ID NO.:5):HLA complex with an IFNγ production pEC₅₀ of between 6.0 and 9.0 (i.e. including 6.0, 9.0, and any value therebetween), between 6.0 and 8.5, between 6.0 and 8.0, between 6.0 and 7.5, between 6.0 and 7.0, between 6.0 and 6.5, between 6.5 and 9.0, between 6.5 and 8.5, between 6.5 and 8.0, between 6.5 and 7.5, between 6.5 and 7.0, between 7.0 and 9.0, between 7.0 and 8.5, between 7.0 and 8.0, between 7.0 and 7.5, between 7.5 and 9.0, between 7.5 and 8.5, between 7.5 and 8.0, between 8.0 and 9.0, between 8.0 and 8.5, or between 8.2 and 9.0.

Embodiment 60. The host cell of any one of Embodiments 48-59, wherein the binding protein is capable of binding to the YLPGAEVPEPA (SEQ ID NO.:5):HLA complex with an IFNγ production pEC₅₀ of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0.

Embodiment 61. The host cell of Embodiment any one of Embodiments 48-60, wherein the binding protein is capable of binding to the YLPGAEVPEPA (SEQ ID NO.:5):HLA complex with an IFNγ production pEC₅₀ of 6.0 or higher.

Embodiment 62. The host cell of any one of Embodiments 48-61, wherein the binding protein is capable of binding to the YLPGAEVPEPA (SEQ ID NO.:5):HLA complex with an IFNγ production pEC₅₀ of 6.5 or higher.

Embodiment 63. The host cell of any one of Embodiments 48-62, wherein the binding protein is capable of binding to the YLPGAEVPEPA (SEQ ID NO.:5):HLA complex with an IFNγ production pEC₅₀ of 7.0 or higher.

Embodiment 64. The host cell of any one of Embodiments 48-63, wherein the binding protein is capable of binding to the YLPGAEVPEPA (SEQ ID NO.:5):HLA complex with an IFNγ production pEC₅₀ of 7.5 or higher.

Embodiment 65. The host cell of any one of Embodiments 48-64, wherein the binding protein is capable of binding to the YLPGAEVPEPA (SEQ ID NO.:5):HLA complex with an IFNγ production pEC₅₀ of 8.0 or higher.

Embodiment 66. The host cell of any one of Embodiments 48-65, wherein the host cell expresses CD137 when in the presence of cells of any one or more of the following tumor cell lines: CFPAC1, H441, Panc08.13, SW620, SW527, L363, MM1R expressing HLA-A2, and INA6 expressing HLA-A2, wherein, optionally, CD137 expression is assessed by flow cytometry of the host cell following incubation of the host cell with the one or more cells of the tumor cell line or lines.

Embodiment 67. The host cell of any one of Embodiments 48-66, wherein, of a plurality of the host cells present in a sample, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, or 80% or more of the plurality of the host cells are positive for expression of CD137 following an incubation with any one or more of the following tumor cell lines: CFPAC1, H441, Panc08.13, SW620, SW527, L363, MM1R expressing HLA-A2, INA6 expressing HLA-A2.

Embodiment 68. The host cell of Embodiment 67, wherein the incubation comprises a duration of about 16 hours to about 18 hours, optionally wherein the incubation comprises a duration of between 16 and 18 hours.

Embodiment 69. The host cell of any one of Embodiments 66-68, wherein, prior to the incubation, the cells of the tumor cell line were administered an agent to increase HLA-A2 expression in the cells of the tumor cell line, wherein, optionally, the agent comprises IFN-γ.

Embodiment 70. A composition, comprising: (i) the binding protein of any one of Embodiments 1-29; (ii) the polynucleotide of any one of Embodiments 30-40; (iii) the vector of any one of Embodiments 41-47; and/or (iv) the host cell of any one of Embodiments 48-49, optionally comprising CD4+ T cells, CD8+ T cells, or both, and a pharmaceutically acceptable carrier, excipient, or diluent.

Embodiment 71. The composition of Embodiment 70, comprising the host cell, wherein the host cell comprises an immune cell, optionally CD8+ T cells and/or CD4+ T cells, wherein, further optionally, the CD8+ T cells and CD4+ T cells are present in about a 1:1 ratio, and/or the composition comprises substantially no naïve T cells.

Embodiment 72. A method for treating a disease or disorder associated with expression of a SOX2 antigen comprising or consisting of the amino acid sequence according to any one of SEQ ID NOs:2-7, or with SOX2 expression or activity, in a subject, the method comprising administering to the subject an effective amount of: (i) the binding protein of any one of Embodiments 1-29; (ii) the polynucleotide of any one of Embodiments 30-40; (iii) the vector of any one of Embodiments 41-47; (iv) the host cell of any one of Embodiments 48-49; and/or (v) the composition of Embodiment 70 or 71,

thereby treating the disease or condition.

Embodiment 73. A method for inducing an immune response in a subject having, and/or for treating in a subject, a disease or disorder associated with expression of a SOX2 antigen comprising or consisting of the amino acid sequence according to any one of SEQ ID NOs:2-7, or with SOX2 expression or activity, the method comprising administering to the subject an effective amount of T cells that express a TCR that specifically binds to a peptide:HLA complex expressed on the surface of a target cell, wherein the peptide comprises or consists of the the amino acid sequence according to any one of SEQ ID NOs:2-7, and wherein the HLA is optionally HLA-A*02:01.

Embodiment 74. The binding protein of any one of Embodiments 1-29, the polynucleotide of any one of Embodiments 30-40, the vector of any one of Embodiments 41-47, the host cell of any one of Embodiments 48-49; and/or the composition of Embodiment 70 or 71, for use in a method of treating a disease or disorder associated with expression of a SOX2 antigen comprising or consisting of the amino acid sequence according to any one of SEQ ID NOs:2-7, or with SOX2 expression or activity, in a subject.

Embodiment 75. The binding protein of any one of Embodiments 1-29, the polynucleotide of any one of Embodiments 30-40, the vector of any one of Embodiments 41-47, the host cell of any one of Embodiments 48-49; and/or the composition of Embodiment 70 or 71, for use in the manufacture of a medicament for treating a disease or disorder associated with expression of a SOX2 antigen comprising or consisting of the amino acid sequence according to any one of SEQ ID NOs:2-7, or with SOX2 expression or activity, in a subject.

Embodiment 76. The method of Embodiment 72 or 73, or the binding protein, polynucleotide, vector, host cell, or composition for use of Embodiment 74 or 75, wherein the subject is HLA-A*02:01*.

Embodiment 77. The method of any one of Embodiments 72, 73, or 76, or 72 or 73, or the binding protein, polynucleotide, vector, host cell, or composition for use of any one of Embodiments 74-76, wherein the disease or condition is a cancer.

Embodiment 78. The method of Embodiment 77 or the binding protein, polynucleotide, vector, host cell, or composition for use of Embodiment 77, wherein the cancer comprises a hematological malignancy or a solid tumor.

Embodiment 79. The method of Embodiment 77 or 78 or the binding protein, polynucleotide, vector, host cell, or composition for use of Embodiment 77 or 78, wherein the cancer comprises multiple myeloma, plasma cell leukemia, ovarian cancer, glioma, lung cancer, neck cancer, cervical cancer, or any combination thereof.

Embodiment 80. The method of any one of Embodiments 72, 73, or 76-79, or the binding protein, polynucleotide, vector, host cell, or composition for use of any one of Embodiments 74-79, wherein the subject is human.

Embodiment 81. The method of any one of Embodiments 72, 73, or 76-80, or the binding protein, polynucleotide, vector, host cell, or composition for use of any one of Embodiments 74-80, wherein the subject has previously received one or more of: (i) surgery; (ii) radiation therapy; (iii) chemotherapy; (iv) a hematopoietic stem cell transplant (HSC); and (v) an adoptive cell therapy, optionally comprising T cells expressing a CAR,

and wherein the disease or disorder is optionally refractory to a prior therapy.

Embodiment 82. The method of any one of Embodiments 72, 73, or 76-81, or the binding protein, polynucleotide, vector, host cell, or composition for use of any one of Embodiments 74-81, wherein one or more of the host cells comprised in the composition is autologous to the subject.

Embodiment 83. The method of any one of Embodiments 72, 73, or 76-82, or the binding protein, polynucleotide, vector, host cell, or composition for use of any one of Embodiments 74-81, further comprising administering an inhibitor of an immune checkpoint molecule to the subject.

Embodiment 84. An immunogenic composition, comprising: (i) an isolated peptide or polypeptide comprising or consisting of the amino acid sequence of SEQ ID NO:2;
(ii) an isolated peptide or polypeptide comprising or consisting of the amino acid sequence of SEQ ID NO:3; (iii) an isolated peptide or polypeptide comprising or consisting of the amino acid sequence of SEQ ID NO:4; (iv) an isolated peptide or polypeptide comprising or consisting of the amino acid sequence of SEQ ID NO:5; (v) an isolated peptide or polypeptide comprising or consisting of the amino acid sequence of SEQ ID NO:6; (vi) an isolated peptide or polypeptide comprising or consisting of the amino acid sequence of SEQ ID NO:7; and/or (vii) a variant of the isolated peptide or polypeptide of any one of (i)-(vi) having one, two, or three amino acid differences as compared to SEQ ID NO:2, 3, 4, 5, 6, or 7, wherein the isolated peptide or polypeptide of any one of (i)-(vii) does not comprise an isolated full-length human SOX2.

Embodiment 85. The immunogenic composition of Embodiment 84, wherein (a) one or more copies of any one of (i)-(vii) and/or (b) one or more of any of (i)-(vii)
is/are present in a fusion polypeptide, wherein the fusion polypeptide optionally further comprises the amino acid sequence of a self-cleaving peptide.

Embodiment 86. The immunogenic composition of Embodiment 84 or 85, wherein the immunogenic composition is capable of eliciting an immune response in a subject against cancer cells, wherein, optionally, the cancer cells comprise multiple myeloma cells, plasma cell leukemia cells, and/or ovarian cancer cells.

Embodiment 87. The immunogenic composition of any one of Embodiments 84-86, further comprising an adjuvant.

Embodiment 88. An isolated polynucleotide encoding: (i) a peptide or polypeptide comprising or consisting of the amino acid sequence of SEQ ID NO:2; (ii)
a peptide or polypeptide comprising or consisting of the amino acid sequence of SEQ ID NO:3; (iii) a peptide or polypeptide comprising or consisting of the amino acid sequence of SEQ ID NO:4; (iv) a peptide or polypeptide comprising or consisting of the amino acid sequence of SEQ ID NO:5; (v) a peptide or polypeptide comprising or consisting of the amino acid sequence of SEQ ID NO:6; (vi) a peptide or polypeptide comprising or consisting of the amino acid sequence of SEQ ID NO:7; and/or (vii) a variant of the peptide or polypeptide of any one of (i)-(vii) having one, two, or three amino acid differences as compared to SEQ ID NO:2, 3, 4, 5, 6, or 7, wherein the polynucleotide is optionally contained in a vector and/or the peptide or polypeptide of any one of (i)-(vii) does not comprise a full-length human SOX2.

Embodiment 89. The polynucleotide of Embodiment 88, wherein the polynucleotide is codon-optimized for expression in a host cell, wherein the host cell is optionally a dendritic cell or a T cell.

Embodiment 90. A host cell comprising the polynucleotide of Embodiment 88 or 89, wherein the polynucleotide is heterologous to the host cell, and wherein the host cell is optionally an immune cell and is further optionally a professional antigen-presenting cell.

Embodiment 91. The host cell of Embodiment 90, wherein the host cell is a dendritic cell or a T cell.

Embodiment 92. A method of eliciting an immune response in a subject against a disease or disorder associated with SOX2 expression or activity, the method comprising administering to the subject the binding protein of any one of Embodiments 1-29, the polynucleotide of any one of Embodiments 30-40, the vector of any one of Embodiments 41-48, the host cell of any one of Embodiments 49-69, the composition of Embodiment 70 or 71, the immunogenic composition of any one of Embodiments 84-87, the polynucleotide of Embodiment 88 or 89, and/or the host cell of Embodiment 90 or 91.

Embodiment 93. A method for expanding a population of T cells that bind to a peptide (e.g., a peptide comprised in a peptide:HLA complex) selected from: (i) a peptide comprising or consisting of the amino acid sequence of SEQ ID NO:2; (ii) a peptide comprising or consisting of the amino acid sequence of SEQ ID NO:3; (iii) a peptide comprising or consisting of the amino acid sequence of SEQ ID NO:4; (iv) a peptide comprising or consisting of the amino acid sequence of SEQ ID NO:5; (v)
a peptide comprising or consisting of the amino acid sequence of SEQ ID NO:6; and/or
   (vi) a peptide comprising or consisting of the amino acid sequence of SEQ ID NO:7,
the method comprising contacting a sample comprising one or more T cells that bind to the peptide with the immunogenic composition of any one of Embodiments 84-87, the polynucleotide of Embodiment 88 or 89, the host cell of Embodiment 90 or 91, and/or antigen-presenting cells that have been contacted with a peptide or polypeptide comprising or consisting of the amino acid sequence set forth in any one of SEQ ID NOS:2-7.

Embodiment 94. A method for generating and/or isolating T cells, the method comprising contacting a sample comprising T cells, wherein the sample optionally comprises peripheral blood cells, with: (i) the immunogenic composition of any one of Embodiments 84-87; (ii) the polynucleotide of Embodiment 88 or 89; (iii)
the host cell of Embodiment 90 or 91; and/or (iv) antigen-presenting cells (APCs) that express or have been contacted with a SOX2 antigen comprising or consisting of the amino acid sequence set forth in any one of SEQ ID NOS:2-7, and optionally sorting T cells from other cells in the sample, thereby isolating and/or generating T cells.

Embodiment 95. A T cell isolated and/or generated by the method of Embodiment 94.

The present disclosure also provides the following non-limiting Additional Embodiments:

Additional Embodiment 1. A modified immune cell, comprising a heterologous polynucleotide encoding a binding protein that includes a T cell receptor (TCR) α-chain variable (Vα) domain and a TCR β-chain variable (Vβ) domain, wherein the encoded binding protein is capable of specifically binding to a SOX2 antigen:HLA complex, wherein the SOX2 antigen comprises or consists of the amino acid sequence set forth in SEQ ID NO.:2, 3, 4, 5, 6, or 7.

Additional Embodiment 2. The modified immune cell of Additional Embodiment 1, wherein the HLA comprises HLA-A*02:01.

Additional Embodiment 3. The modified immune cell of Additional Embodiment 1 or 2, wherein the encoded binding protein comprises: (i) a CDR3β, a CDR2β, and/or a CDR1β of a TCR of a T cell from: Donor 1 Line 1, Donor 1 Line 3, Donor 2 Line 8, Donor 2 Line 1, Donor 2 Line 13, Donor 2 Line 11, or Donor 2 Line 19; and/or (ii) a CDR3α, a CDR2α, and/or a CDR1α of a TCR of a T cell from: Donor 1 Line 1, Donor 1 Line 3, Donor 2 Line 8, Donor 2 Line 1, Donor 2 Line 13, Donor 2 Line 11, or Donor 2 Line 19.

Additional Embodiment 4. The modified immune cell of any one of Additional Embodiments 1-3, wherein the encoded binding protein comprises: (i) a Vα domain having at least 90% amino acid identity to the Vα domain of a TCR of a T cell from: Donor 1 Line 1, Donor 1 Line 3, Donor 2 Line 8, Donor 2 Line 1, Donor 2 Line 13, Donor 2 Line 11, or Donor 2 Line 19; and/or (ii) a Vβ domain having at least 90% amino acid identity to the Vβ domain of a TCR of a T cell from: Donor 1 Line 1, Donor 1 Line 3, Donor 2 Line 8, Donor 2 Line 1, Donor 2 Line 13, Donor 2 Line 11, or Donor 2 Line 19, provided that (a) at least three or four of the CDRs have no mutations; (b) the CDRs that do have mutations have only up to two amino acid substitutions, up to a contiguous five amino acid deletion, or a combination thereof, and (c) the encoded binding protein retains its ability to bind to a SOX2:HLA complex.

Additional Embodiment 5. The modified immune cell of Additional Embodiment 1 or 2, wherein the encoded binding protein comprises: (i) a CDR3βamino acid sequence according to any one of SEQ ID NOs.:16, 17, 28, 29, 40, 41, 52, 53, 64, 65, 76, 77, 88, 89, 100, 101, 112, 113, 124, 125, 136, 137, 148, or 149, or a variant thereof comprising one, two, or three amino acid substitutions; (ii) a CDR2βamino acid sequence according to any one of SEQ ID NOs.:15, 27, 39, 51, 63, 75, 87, 99, 111, 123, 135, or 147, or a variant thereof comprising one, two, or three amino acid substitutions; (iii) a CDR1β amino acid sequence according to any one of SEQ ID NOs.:14, 26, 38, 50, 62, 74, 86, 98, 110, 122, 134, 146, or a variant thereof comprising one, two, or three amino acid substitutions; (iv) a CDR3α amino acid sequence according to any one of SEQ ID NOs.:21, 22, 33, 34, 45, 46, 57, 58, 69, 70, 81, 82, 93, 94, 105, 106, 117, 118, 129, 130, 141, 142, 153, or 154, or a variant thereof comprising one, two, or three amino acid substitutions; (v) a CDR2α amino acid sequence according to any one of SEQ ID NOs.:20, 32, 44, 56, 68, 80, 92, 104, 116, 128, 140, 152, or a variant thereof comprising one, two, or three amino acid substitutions; and/or (vi) a CDR1α amino acid sequence according to any one of SEQ ID NOs.:19, 31, 43, 55, 67, 79, 91, 103, 115, 127, 139, or 151, or a variant thereof comprising one, two, or three amino acid substitutions.

Additional Embodiment 6. The modified immune cell of Additional Embodiment 5, wherein the encoded binding protein comprises CDR1α, CDR2α, CDR3α, CDR1β, CDR2β, and CDR3β amino acid sequences according to: (i) SEQ ID NOs.:19, 20, 21 or 22, 14, 15, and 16 or 17, respectively; (ii) SEQ ID NOs.:31, 32, 33 or 34, 26, 27, and 28 or 29, respectively; (iii) SEQ ID NOs.:43, 44, 45 or 46, 38, 39, and 40 or 41, respectively; (iv) SEQ ID NOs.:55, 56, 57 or 58, 50, 51, and 52 or 53, respectively; (v) SEQ ID NOs.:67, 68, 69 or 70, 62, 63, and 64 or 65, respectively; (vi) SEQ ID NOs.:79, 80, 81 or 82, 74, 75, and 76 or 77, respectively; (vii) SEQ ID NOs.:91, 92, 93 or 94, 86, 87, and 88 or 89, respectively; (viii) SEQ ID NOs.:103, 104, 105 or 106, 98, 99, and 100 or 101, respectively; (ix) SEQ ID NOs.:115, 116, 117 or 118, 110, 111, and 112 or 113, respectively; (x) SEQ ID NOs.:127, 128, 129 or 130, 122, 123, and 124 or 125, respectively; (xi) SEQ ID NOs.:139, 140, 141 or 142, 134, 135, and 136 or 137 respectively; or (xii) SEQ ID NOs.:151, 152, 153 or 154, 146, 147, and 148 or 149, respectively.

Additional Embodiment 7. The modified immune cell of Additional Embodiment 1, 2, 5, or 6, wherein the encoded Vα domain comprises or consists of an amino acid sequence having at least 80%, 85%, 90%, 91%, 92%, 93%, 94%, 95%, 96%, 97%, 98%, or 99% identity to the amino acid sequence set forth in any one of SEQ ID NOs.:18, 30, 42, 54, 66, 78, 90, 102, 114, 126, 138, or 150.

Additional Embodiment 8. The modified immune cell of any one of Additional Embodiments 1, 2, or 5-7, wherein the encoded Vβ domain comprises or consists of the amino acid sequence having at least 80%, 85%, 90%, 91%, 92%, 93%, 94%, 95%, 96%, 97%, 98%, or 99% identity to the amino acid sequence set forth in any one of SEQ ID NOs.:13, 25, 37, 49, 61, 73, 85, 97, 109, 121, 133, or 145.

Additional Embodiment 9. The modified immune cell of any one of Additional Embodiments 5-8, wherein the encoded Vα domain and the encoded Vβ domain comprise or consist of the amino acid sequences according to: (i) SEQ ID NOs.:18 and 13, respectively; (ii) SEQ ID NOs.:30 and 25, respetively; (iii) SEQ ID NOs.:42 and 37, respectively; (iv) SEQ ID NOs.:54 and 49, respectively; (v) SEQ ID NOs.:66 and 61, respectively; (vi) SEQ ID NOs.:78 and 73, respectively; (vii) SEQ ID NOs.:90 and 85, respectively; (viii) SEQ ID NOs.:102 and 97, respectively; (ix) SEQ ID NOs.:114 and 109, respectively; (x) SEQ ID NOs.:126 and 121, respectively; (xi) SEQ ID NOs.:138 and 133 respectively; or (xii) SEQ ID NOs.:150 and 145, respectively.

Additional Embodiment 10. The modified immune cell of any one of Additional Embodiments 1-9, wherein the encoded binding protein is comprised in an amino acid sequence having at least 90%, 91%, 92%, 93%, 94%, 95%, 96%, 97%, 98%, or 99% identity to the amino acid sequence set forth in any one of SEQ ID NOs.:12, 24, 36, 48, 60, 72, 84, 96, 108, 120, 132, or 144.

Additional Embodiment 11. The modified immune cell of any one of Additional Embodiments 1-10, wherein the polynucleotide encoding a binding protein comprises a polynucleotide having at least 70%, 75%, 80%, 85%, 90%, 91%, 92%, 93%, 94%, 95%, 96%, 97%, 98%, or 99% identity to the nucleotide acid sequence set forth in any one of SEQ ID NOs.:11, 23, 35, 47, 59, 71, 83, 95, 107, 119, 131, or 143.

Additional Embodiment 12. The modified immune cell of any one of Additional Embodiments 1-11, wherein the encoded binding protein comprises a TCR, a single-chain TCR (scTCR), a scTv, a chimeric antigen receptor (CAR), or any combination thereof.

Additional Embodiment 13. The modified immune cell of any one of Additional Embodiments 1-12, further comprising a polynucleotide that encodes a TCR β-polypeptide constant domain (Cβ), a polynucleotide that encodes a TCR α-polypeptide constant domain (Cα), or both.

Additional Embodiment 14. The modified immune cell of Additional Embodiment 13, comprising a polynucleotide that encodes a TCR Cβ and a polynucleotide that encodes a TCR Cα, wherein the encoded TCR Cβ comprises a cysteine amino acid at amino acid position 57, and wherein the encoded TCR Cα comprises a cysteine amino acid at amino acid position 48.

Additional Embodiment 15. The modified immune cell of any one of Additional Embodiments 1-14, wherein the immune cell comprises a T cell, a NK cell, a NK-T cell, or any combination thereof.

Additional Embodiment 16. The modified immune cell of Additional Embodiment 15, wherein the immune cell comprises a CD8⁺ T cell and/or a CD4⁺ T cell.

Additional Embodiment 17. The modified immune cell of any one of Additional Embodiments 1-16, wherein the heterologous polynucleotide encoding the binding protein is codon optimized for expression in a host cell.

Additional Embodiment 18. The modified immune cell of any one of Additional Embodiments 1-17, wherein the immune cell comprises a chromosomal gene knockout of a PD-1 gene; a LAG3 gene; a TIM3 gene; a CTLA4 gene; an HLA component gene; a TCR component gene; or any combination thereof.

Additional Embodiment 19. The modified immune cell of Additional Embodiment 18, wherein the chromosomal gene knockout comprises a knockout of an HLA component gene selected from an α1 macroglobulin gene; an α2 macroglobulin gene; an α3 macroglobulin gene; a β1 microglobulin gene; or a β2 microglobulin gene; or any combination thereof.

Additional Embodiment 20. The modified immune cell of Additional Embodiment 18 or 19, wherein the chromosomal gene knockout comprises a knockout of a TCR component gene selected from a TCR α variable region gene; a TCR β variable region gene; a TCR constant region gene; or any combination thereof.

Additional Embodiment 21. A composition, comprising a modified immune cell of any one of Additional Embodiments 1-20 and a pharmaceutically acceptable carrier, diluent, or excipient.

Additional Embodiment 22. A method for treating a disease or disorder associated with expression of a SOX2 antigen comprising or consisting of the amino acid sequence according to any one of SEQ ID NOs:2-7, or with SOX2 expression or activity, in a subject, the method comprising administering to the subject an effective amount of the modified immune cell of any one of Additional Embodiments 1-20 or a composition of Additional Embodiment 21, thereby treating the disease or condition.

Additional Embodiment 23. A method for inducing an immune response in a subject having, and/or for treating in a subject, a disease or disorder associated with expression of a SOX2 antigen comprising or consisting of the amino acid sequence according to any one of SEQ ID NOs:2-7, or with SOX2 expression or activity, the method comprising administering to the subject an effective amount of T cells that express a TCR that specifically binds to a peptide:HLA complex expressed on the surface of a target cell, wherein the peptide comprises or consists of the the amino acid sequence according to any one of SEQ ID NOs:2-7, and wherein the HLA is optionally HLA-A*02:01.

Additional Embodiment 24. The method of Additional Embodiment 22 or 23, wherein the subject is HLA-A*02:01*.

Additional Embodiment 25. The method of any one of Additional Embodiments 22-24, wherein the disease or condition is a cancer.

Additional Embodiment 26. The method of Additional Embodiment 25, wherein the cancer comprises a hematological malignancy or a solid tumor.

Additional Embodiment 27. The method of Additional Embodiment 25 or 26, wherein the cancer comprises multiple myeloma, plasma cell leukemia, or ovarian cancer.

Additional Embodiment 28. The method of any one of Additional Embodiments 22-27, wherein the subject is human.

Additional Embodiment 29. The method of any one of Additional Embodiments 22-28, wherein the subject has previously received one or more of: (i) surgery;
(ii) radiation therapy; (iii) chemotherapy; (iv) a hematopoietic stem cell transplant (HSC); or (v) an adoptive cell therapy, optionally comprising T cells expressing a CAR, and wherein the disease or disorder is optionally refractory to a prior therapy.

Additional Embodiment 30. The method of any one of Additional Embodiments 22-29, wherein one or more of the modified immune cells comprised in the composition is autologous to the subject.

Additional Embodiment 31. The method of any one of Additional Embodiments 22-30, further comprising administering an inhibitor of an immune checkpoint molecule to the subject.

Additional Embodiment 32. An isolated polynucleotide encoding a binding protein that includes a T cell receptor (TCR) α polypeptide variable (Vα) domain and a TCR β-polypeptide variable (Vβ) domain, wherein the encoded binding protein is capable of specifically binding to a SOX2 antigen:HLA complex, wherein the SOX2 antigen comprises or consists of the amino acid sequenceset forth in SEQ ID NO:2, 3, 4, 5, 6, or 7, wherein the polynucleotide is optionally codon optimized for expression in a host cell.

Additional Embodiment 33. The isolated polynucleotide of Additional Embodiment 32, wherein the HLA comprises HLA-A*02:01.

Additional Embodiment 34. The isolated polynucleotide of Additional Embodiment 33, wherein the encoded binding protein comprises: (i) a CDR3β, a CDR2β, and/or a CDR1β of a TCR of a T cell from: Donor 1 Line 1, Donor 1 Line 3, Donor 2 Line 8, Donor 2 Line 1, Donor 2 Line 13, Donor 2 Line 11, or Donor 2 Line 19; and/or (ii) a CDR3α, a CDR2α, and/or a CDR1α of a TCR of a T cell from: Donor 1 Line 1, Donor 1 Line 3, Donor 2 Line 8, Donor 2 Line 1, Donor 2 Line 13, Donor 2 Line 11, or Donor 2 Line 19.

Additional Embodiment 35. The isolated polynucleotide of any one of Additional Embodiments 32-34, wherein the encoded binding protein comprises: (i)
a Vα domain having at least 90% amino acid identity to the Vα domain of a TCR of a T cell from: Donor 1 Line 1, Donor 1 Line 3, Donor 2 Line 8, Donor 2 Line 1, Donor 2 Line 13, Donor 2 Line 11, or Donor 2 Line 19; and/or (ii) a Vβ domain having at least 90% amino acid identity to the Vβ domain of a TCR of a T cell from: Donor 1 Line 1, Donor 1 Line 3, Donor 2 Line 8, Donor 2 Line 1, Donor 2 Line 13, Donor 2 Line 11, or Donor 2 Line 19, provided that (a) at least three or four of the CDRs have no mutations; (b) the CDRs that do have mutations have only up to two amino acid substitutions, up to a contiguous five amino acid deletion, or a combination thereof, and (c) the encoded binding protein retains its ability to bind to a SOX2:HLA complex.

Additional Embodiment 36. The isolated polynucleotide of Additional Embodiment 32 or 33, wherein the encoded binding protein comprises: (i) a CDR3βamino acid sequence according to any one of SEQ ID NOs.:16, 17, 28, 29, 40, 41, 52, 53, 64, 65, 76, 77, 88, 89, 100, 101, 112, 113, 124, 125, 136, 137, 148, or 149, or a variant thereof comprising one, two, or three amino acid substitutions; (ii) a CDR2βamino acid sequence according to any one of SEQ ID NOs.:15, 27, 39, 51, 63, 75, 87, 99, 111, 123, 135, or 147, or a variant thereof comprising one, two, or three amino acid substitutions; (iii) a CDR1β amino acid sequence according to any one of SEQ ID NOs.:14, 26, 38, 50, 62, 74, 86, 98, 110, 122, 134, 146, or a variant thereof comprising one, two, or three amino acid substitutions; (iv) a CDR3α amino acid sequence according to any one of SEQ ID NOs.:21, 22, 33, 34, 45, 46, 57, 58, 69, 70, 81, 82, 93, 94, 105, 106, 117, 118, 129, 130, 141, 142, 153, or 154, or a variant thereof comprising one, two, or three amino acid substitutions;
(v) a CDR2α amino acid sequence according to any one of SEQ ID NOs.:20, 32, 44, 56, 68, 80, 92, 104, 116, 128, 140, 152, or a variant thereof comprising one, two, or three amino acid substitutions; and/or (vi) a CDR1α amino acid sequence according to any one of SEQ ID NOs.:19, 31, 43, 55, 67, 79, 91, 103, 115, 127, 139, or 151, or a variant thereof comprising one, two, or three amino acid substitutions.

Additional Embodiment 37. The isolated polynucleotide of Additional Embodiment 36, wherein the encoded binding protein comprises CDR1α, CDR2α, CDR3α, CDR1β, CDR2β, and CDR3β amino acid sequences according to: (i) SEQ ID NOs.:19, 20, 21 or 22, 14, 15, and 16 or 17, respectively; (ii) SEQ ID NOs.:31, 32, 33 or 34, 26, 27, and 28 or 29, respectively; (iii) SEQ ID NOs.:43, 44, 45 or 46, 38, 39, and 40 or 41, respectively; (iv) SEQ ID NOs.:55, 56, 57 or 58, 50, 51, and 52 or 53, respectively; (v) SEQ ID NOs.:67, 68, 69 or 70, 62, 63, and 64 or 65, respectively; (vi) SEQ ID NOs.:79, 80, 81 or 82, 74, 75, and 76 or 77, respectively; (vii) SEQ ID NOs.:91, 92, 93 or 94, 86, 87, and 88 or 89, respectively; (viii) SEQ ID NOs.:103, 104, 105 or 106, 98, 99, and 100 or 101, respectively; (ix) SEQ ID NOs.:115, 116, 117 or 118, 110, 111, and 112 or 113, respectively; (x) SEQ ID NOs.:127, 128, 129 or 130, 122, 123, and 124 or 125, respectively; (xi) SEQ ID NOs.:139, 140, 141 or 142, 134, 135, and 136 or 137 respectively; or (xii) SEQ ID NOs.:151, 152, 153 or 154, 146, 147, and 148 or 149, respectively.

Additional Embodiment 38. The isolated polynucleotide of any one of Additional Embodiments 32, 33, 36, or 37, wherein the encoded Vα domain comprises or consists of an amino acid sequence having at least 80%, 85%, 90%, 91%, 92%, 93%, 94%, 95%, 96%, 97%, 98%, or 99% identity to the amino acid sequence set forth in any one of SEQ ID NOs.:18, 30, 42, 54, 66, 78, 90, 102, 114, 126, 138, or 150.

Additional Embodiment 39. The isolated polynucleotide of any one of Additional Embodiments 32, 33, or 36-38, wherein the encoded Vβ domain comprises or consists of the amino acid sequence having at least 80%, 85%, 90%, 91%, 92%, 93%, 94%, 95%, 96%, 97%, 98%, or 99% identity to the amino acid sequence set forth in any one of SEQ ID NOs.:13, 25, 37, 49, 61, 73, 85, 97, 109, 121, 133, or 145.

Additional Embodiment 40. The isolated polynucleotide of any one of Additional Embodiments 36-39, wherein the encoded Vα domain and the encoded Vβ domain comprise or consist of the amino acid sequences according to: (i) SEQ ID NOs.:18 and 13, respectively; (ii) SEQ ID NOs.:30 and 25, respectively; (iii) SEQ ID NOs.:42 and 37, respectively; (iv) SEQ ID NOs.:54 and 49, respectively; (v) SEQ ID NOs.:66 and 61, respectively; (vi) SEQ ID NOs.:78 and 73, respectively; (vii) SEQ ID NOs.:90 and 85, respectively; (viii) SEQ ID NOs.:102 and 97, respectively; (ix) SEQ ID NOs.:114 and 109, respectively; (x) SEQ ID NOs.:126 and 121, respectively; (xi) SEQ ID NOs.:138 and 133 respectively; or (xii) SEQ ID NOs.:150 and 145, respectively.

Additional Embodiment 41. The isolated polynucleotide of any one of Additional Embodiments 32-40, encoding an amino acid sequence having at least 90%, 91%, 92%, 93%, 94%, 95%, 96%, 97%, 98%, 99%, or 100% identity to the amino acid sequence set forth in any one of SEQ ID NOs.:12, 24, 36, 48, 60, 72, 84, 96, 108, 120, 132, or 144.

Additional Embodiment 42. The isolated polynucleotide of any one of Additional Embodiments 32-41, comprising a polynucleotide having at least 70%, 75%, 80%, 85%, 90%, 91%, 92%, 93%, 94%, 95%, 96%, 97%, 98%, 99%, or 100% identity to the nucleotide acid sequence set forth in any one of SEQ ID NOs.:11, 23, 35, 47, 59, 71, 83, 95, 107, 119, 131, or 143.

Additional Embodiment 43. The isolated polynucleotide of any one of Additional Embodiments 32-42, wherein the encoded binding protein comprises a TCR, a single-chain TCR (scTCR), as scTv, a chimeric antigen receptor (CAR), or any combination thereof.

Additional Embodiment 44. The isolated polynucleotide of any one of Additional Embodiments 32-43, further comprising a polynucleotide that encodes a TCR β-polypeptide constant domain (Cβ), a polynucleotide that encodes a TCR α-polypeptide constant domain (Cα), or both.

Additional Embodiment 45. The isolated polynucleotide of Additional Embodiment 44, comprising a polynucleotide that encodes a TCR Cβ and a polynucleotide that encodes a TCR Cα, wherein the encoded TCR Cβ comprises a cysteine amino acid at amino acid position 57, and wherein the encoded TCR Cα comprises a cysteine amino acid at amino acid position 48.

Additional Embodiment 46. The isolated polynucleotide of any one of Additional Embodiments 32-45, wherein the polynucleotide is codon-optimized for expression in an immune cell.

Additional Embodiment 47. The isolated polynucleotide of Additional Embodiment 46, wherein the immune cell is a T cell, a NK cell, or a NK-T cell.

Additional Embodiment 48. The isolated polynucleotide of Additional Embodiment 47, wherein the immune cell comprises a CD8⁺ T cell and/or a CD4⁺ T cell.

Additional Embodiment 49. The isolated polynucleotide of any one of Additional Embodiments 32-48, comprising a polynucleotide encoding a self-cleaving peptide disposed between the Vβ-encoding polynucleotide and the Vα-encoding polynucleotide, or disposed between the TCR β polypeptide-encoding polynucleotide and the TCR α polypeptide-encoding polynucleotide.

Additional Embodiment 50. A vector comprising the isolated polynucleotide of any one of Additional Embodiments 32-49.

Additional Embodiment 51. An immunogenic composition, comprising: (i) a SOX2 antigen comprising or consisting of the amino acid sequence of SEQ ID NO:2; (ii) a SOX2 antigen comprising or consisting of the amino acid sequence of SEQ ID NO:3;
(iii) a SOX2 antigen comprising or consisting of the amino acid sequence of SEQ ID NO:4; (iv) a SOX2 antigen comprising or consisting of the amino acid sequence of SEQ ID NO:5; (v) a SOX2 antigen comprising or consisting of the amino acid sequence of SEQ ID NO:6; (vi) a SOX2 antigen comprising or consisting of the amino acid sequence of SEQ ID NO:7; and/or (vii) a variant of a SOX2 antigen having one, two, or three amino acid differences as compared to SEQ ID NO:2, 3, 4, 5, 6, or 7.

Additional Embodiment 52. The immunogenic composition of Additional Embodiment 51, wherein (a) two or more copies of any one of (i)-(vi) and/or (b) two or more of any of (i)-(vi) are present in a fusion polypeptide, wherein the fusion polypeptide optionally further comprises an amino acid sequence of a self-cleaving peptide.

Additional Embodiment 53. The immunogenic composition of Additional Embodiment 51 or 52, wherein the immunogenic composition is capable of eliciting an immune response against multiple myeloma cells, plasma cell leukemia cells, and/or ovarian cancer cells.

Additional Embodiment 54. The immunogenic composition of any one of Additional Embodiments 51-53, further comprising an adjuvant.

Additional Embodiment 55. An isolated polynucleotide encoding: (i) a SOX2 antigen comprising or consisting of the amino acid sequence of SEQ ID NO:2; (ii) a SOX2 antigen comprising or consisting of the amino acid sequence of SEQ ID NO:3; (iii) a SOX2 antigen comprising or consisting of the amino acid sequence of SEQ ID NO:4;
(iv) a SOX2 antigen comprising or consisting of the amino acid sequence of SEQ ID NO:5; (v) a SOX2 antigen comprising or consisting of the amino acid sequence of SEQ ID NO:6; (vi) a SOX2 antigen comprising or consisting of the amino acid sequence of SEQ ID NO:7; and/or (vii) a variant of a SOX2 antigen having one, two, or three amino acid differences as compared to SEQ ID NO:2, 3, 4, 5, 6, or 7, wherein the polynucleotide is optionally contained in a vector.

Additional Embodiment 56. The isolated polynucleotide of Additional Embodiment 55, wherein the polynucleotide is codon-optimized for expression in a host cell, wherein the host cell is optionally a dendritic cell or a T cell.

Additional Embodiment 57. A host cell comprising the isolated polynucleotide of Additional Embodiment 47 or 48, wherein the host cell is optionally an immune cell and is further optionally a professional antigen-presenting cell.

Additional Embodiment 58. The host cell of Additional Embodiment 57, wherein the host cell is a dendritic cell or a T cell.

Additional Embodiment 59. A method of eliciting an immune response in a subject against a disease or disorder associated with SOX2 expression or activity, the method comprising administering to the subject the modified immune cell of any one of Additional Embodiments 1-20, the composition of Additional Embodiment 21, the immunogenic composition of any one of Additional Embodiments 51-54, the polynucleotide of Additional Embodiment 55 or 56, and/or the host cell of Additional Embodiment 57 or 58.

Additional Embodiment 60. A method for expanding a population of T cells that specifically bind to a SOX2 antigen selected from: (i) a SOX2 antigen comprising or consisting of the amino acid sequence of SEQ ID NO:2; (ii) a SOX2 antigen comprising or consisting of the amino acid sequence of SEQ ID NO:3; (iii) a SOX2 antigen comprising or consisting of the amino acid sequence of SEQ ID NO:4; (iv) a SOX2 antigen comprising or consisting of the amino acid sequence of SEQ ID NO:5; (v) a SOX2 antigen comprising or consisting of the amino acid sequence of SEQ ID NO:6; and/or (vi) a SOX2 antigen comprising or consisting of the amino acid sequence of SEQ ID NO:7,
the method comprising contacting a sample comprising one or more T cells that specifically bind to the antigen with the immunogenic composition of any one of Additional Embodiments 51-54, the polynucleotide of Additional Embodiment 55 or 56, the host cell of Additional Embodiment 57 or 58, and/or antigen-presenting cells that have been pulsed with a SOX2 antigen comprising or consisting of the amino acid sequence set forth in any one of SEQ ID NOS:2-7.

Additional Embodiment 61. A method for generating and/or isolating T cells, the method comprising contacting peripheral blood cells with: (i) the immunogenic composition of any one of Additional Embodiments 51-54; (ii) the polynucleotide of Additional Embodiment 55 or 56; (iii) the host cell of Additional Embodiment 57 or 58; and/or (iv) antigen-presenting cells (APCs) that have been pulsed or otherwise contacted with a SOX2 antigen comprising or consisting of the amino acid sequence set forth in any one of SEQ ID NOS:2-7, and optionally sorting T cells from the peripheral blood cells, thereby isolating and/or generating T cells.

SEQUENCES
(human SOX2 amino acid sequence)
SEQ ID NO.: 1
MYNMMETELK PPGPQQTSGG GGGNSTAAAA GGNQKNSPDR VKRPMNAFMV
WSRGQRRKMA QENPKMHNSE ISKRLGAEWK LLSETEKRPF IDEAKRLRAL
HMKEHPDYKY RPRRKTKTLM KKDKYTLPGG LLAPGGNSMA SGVGVGAGLG
AGVNQRMDSY AHMNGWSNGS YSMMQDQLGY PQHPGLNAHG
AAQMQPMHRY DVSALQYNSM TSSQTYMNGS PTYSMSYSQQ
GTPGMALGSM GSVVKSEASS SPPVVTSSSH SRAPCQAGDL RDMISMYLPG
AEVPEPAAPS RLHMSQHYQS GPVPGTAING TLPLSHM
(SOX2 peptide (275-283))
SEQ ID NO.: 2
SMYLPGAEV
(SOX2 peptide (216-225))
SEQ ID NO.: 3
YMNGSPTYSM
(SOX2 peptide (306-314))
SEQ ID NO.: 4
TAINGTLPL
(SOX2 peptide (277-287))
SEQ ID NO.: 5
YLPGAEVPEPA
(SOX2 peptide (58-66))
SEQ ID NO.: 6
KMAQENPKM
(SOX2 peptide (118-127))
SEQ ID NO.: 7
TLMKKDKYTL
(hTCR_Calpha-R)
SEQ ID NO.: 8
CAGCCGCAGCGTCATGAGCAGATTA
(hTCR_Cb1-R)
SEQ ID NO.: 9
CCACTTCCAGGGCTGCCTTCAGAAATC
(hTCR_Cb2-R)
SEQ ID NO.: 10
TGGGATGGTTTTGGAGCTAGCCTCTGG
Sequence Legend-color coding:
Leader sequence
V-beta
CDR1, CDR2-beta
CDR3-beta; junction
J-beta
C-beta
P2A
Leader sequence
V-alpha
CDR1, CDR2-alpha
CDR3-alpha; junction
J-alpha
C-alpha
(SOX2 TCR#01 TCRβ-P2A-TCRα, codon-optimized nucleotide)
SEQ ID NO.: 11
ATGGCTTCTCTCCTGTTCTTCTGTGGCGCCTTTTACCTGCTGGGAACAGGCTCTA
TGGATGCTGATGTGACACAGACCCCCAGAAACAGAATCACCAAGACAGGCA
AGCGGATCATGCTGGAGTGTAGCCAGACAAAAGGCCACGACCGGATGTACT
GGTATAGACAAGATCCTGGGTTGGGCCTTCGACTGATCTACTACAGCTTCGA
CGTGAAGGACATCAACAAGGGCGAGATCTCTGACGGCTACAGCGTGAGCA
GACAAGCTCAGGCCAAGTTTTCTCTGTCTCTGGAATCTGCCATCCCTAATCA
GACAGCCCTGTACTTCTGTGCCACCAGCGAGTGGGATAGCACAGATACACA
GTACTTTGGCCCCGGCACCAGGCTGACAGTGCTTGAGGACCTGAAGAACGT
GTTCCCCCCAGAGGTGGCCGTGTTCGAGCCTAGCGAGGCCGAGATCAGCCA
CACCCAGAAAGCCACCCTCGTGTGCCTGGCCACCGGCTTTTACCCCGACCAC
GTGGAACTGTCTTGGTGGGTCAACGGCAAAGAGGTGCACAGCGGCGTCTGC
ACCGACCCCCAGCCCCTGAAAGAGCAGCCCGCCCTGAACGACAGCCGGTAC
TGTCTGAGCAGCAGACTGAGAGTGTCCGCCACCTTCTGGCAGAACCCCCGG
AACCACTTCAGATGCCAGGTGCAGTTCTACGGCCTGAGCGAGAACGACGAG
TGGACCCAGGACCGGGCCAAGCCCGTGACCCAGATCGTGTCTGCTGAGGCC
TGGGGCAGAGCCGATTGCGGCTTCACCAGCGAGAGCTACCAGCAGGGCGTG
CTGAGCGCCACCATCCTGTACGAGATCCTGCTGGGCAAGGCCACCCTGTAC
GCCGTGCTGGTGTCCGCCCTGGTGCTGATGGCCATGGTCAAGCGGAAGGAC
AGCCGGGGCGGTTCCGGAGCCACGAACTTCTCTCTGTTAAAGCAAGCAGGA
GACGTGGAAGAAAACCCCGGTCCCATGGAGACACTGCTTGGCGTATCACTGGT
GATTCTGTGGCTGCAACTGGCTAGAGTGAACTCTCAGCAGGGAGAAGAGGATC
CTCAAGCTCTGAGCATTCAGGAAGGCGAAAACGCTACCATGAATTGCTCCT
ACAAGACCAGCATCAACAACCTGCAGTGGTACCGGCAGAATAGCGGAAGA
GGACTGGTTCACCTGATCCTGATCAGAAGCAATGAAAGGGAGAAGCACAGC
GGCAGACTGAGAGTTACCCTGGATACATCCAAGAAAAGTAGCTCTCTGCTG
ATTACAGCCTCTAGAGCCGCCGATACAGCCAGCTACTTTTGTGCCACATTAG
GACAGGGAGCCCAGAAACTGGTGTTTGGACAGGGCACAAGACTGACCATC
AATCCCAACATCCAGAATCCTGATCCCGCCGTGTACCAGCTGCGGGACAGC
AAGAGCAGCGACAAGAGCGTGTGCCTGTTCACCGACTTCGACAGCCAGACC
AACGTGTCCCAGAGCAAGGACAGCGACGTGTACATCACCGATAAGTGCGTG
CTGGACATGCGGAGCATGGACTTCAAGAGCAACAGCGCCGTGGCCTGGTCC
AACAAGAGCGACTTCGCCTGCGCCAACGCCTTCAACAACAGCATTATCCCC
GAGGACACATTCTTCCCAAGCCCCGAGAGCAGCTGCGACGTGAAGCTGGTG
GAAAAGAGCTTCGAGACAGACACCAACCTGAACTTCCAGAACCTCAGCGTG
ATCGGCTTCCGGATCCTGCTGCTGAAGGTGGCCGGCTTCAACCTGCTGATGA
CCCTGCGGCTGTGGTCCAGCTGA
(SOX2 TCR#01 TCRβ-P2A-TCRα, amino acid)
SEQ ID NO.: 12
MASLLFFCGAFYLLGTGSMDADVTQTPRNRITKTGKRIMLECSQTKGHDRMYW
YRQDPGLGLRLIYYSFDVKDINKGEISDGYSVSRQAQAKFSLSLESAIPNQTALY
FCATSEWDSTDTQYFGPGTRLTVLEDLKNVFPPEVAVFEPSEAEISHTQKATLV
CLATGFYPDHVELSWWVNGKEVHSGVCTDPQPLKEQPALNDSRYCLSSRLRVS
ATFWQNPRNHFRCQVQFYGLSENDEWTQDRAKPVTQIVSAEAWGRADCGFTS
ESYQQGVLSATILYEILLGKATLYAVLVSALVLMAMVKRKDSRGGSGATNFSL
LKQAGDVEENPGPMETLLGVSLVILWLQLARVNSQQGEEDPQALSIQEGENATM
NCSYKTSINNLQWYRQNSGRGLVHLILIRSNEREKHSGRLRVTLDTSKKSSSLLI
TASRAADTASYFCATLGQGAQKLVFGQGTRLTINPNIQNPDPAVYQLRDSKSSD
KSVCLFTDFDSQTNVSQSKDSDVYITDKCVLDMRSMDFKSNSAVAWSNKSDF
ACANAFNNSIIPEDTFFPSPESSCDVKLVEKSFETDTNLNFQNLSVIGFRILLLKV
AGFNLLMTLRLWSS
(SOX2 TCR#01 TCRVβ, amino acid)
SEQ ID NO.: 13
DADVTQTPRNRITKTGKRIMLECSQTKGHDRMYWYRQDPGLGLRLIYYSFDVK
DINKGEISDGYSVSRQAQAKFSLSLESAIPNQTALYFCATSEWDSTDTQYFGPGT
RLTVLE
(SOX2 TCR#01 CDR1β, amino acid)
SEQ ID NO.: 14
KGHDR
(SOX2 TCR#01 CDR2β, amino acid)
SEQ ID NO.: 15
SFDVKD
(SOX2 TCR#01 CDR3β, amino acid)
SEQ ID NO.: 16
ATSEWDSTDTQY
(SOX2 TCR#01 CDR3β-junction, amino acid)
SEQ ID NO.: 17
CATSEWDSTDTQYF
(SOX2 TCR#01 TCRVα, amino acid)
SEQ ID NO.: 18
SQQGEEDPQALSIQEGENATMNCSYKTSINNLQWYRQNSGRGLVHLILIRSNER
EKHSGRLRVTLDTSKKSSSLLITASRAADTASYFCATLGQGAQKLVFGQGTRLT
INPN
(SOX2 TCR#01 CDR1α, amino acid)
SEQ ID NO.: 19
TSINN
(SOX2 TCR#01 CDR2α, amino acid)
SEQ ID NO.: 20
IRSNERE
(SOX2 TCR#01 CDR3α, amino acid)
SEQ ID NO.: 21
ATLGQGAQKLV
(SOX2 TCR#01 CDR3α-junction, amino acid)
SEQ ID NO.: 22
CATLGQGAQKLVF
(SOX2 TCR#02 TCRβ-P2A-TCRα, codon-optimized nucleotide)
SEQ ID NO.: 23
ATGGGCATCAGACTCCTTTGCAGGGTGGCCTTTTGCTTTCTGGCTGTGGGATTAG
TGGACGTGAAGGTTACCCAGTCCAGTAGATACCTTGTCAAGAGAACCGGCG
AGAAGGTGTTCCTGGAATGTGTGCAGGACATGGATCACGAGAATATGTTCT
GGTACAGACAGGACCCTGGACTGGGACTGAGACTGATCTACTTCAGCTACG
ACGTGAAGATGAAGGAGAAGGGCGATATCCCTGAGGGCTACAGCGTGAGC
AGAGAGAAAAAGGAGAGGTTCAGCCTGATTCTGGAAAGCGCCAGCACAAA
TCAAACATCTATGTATTTATGCGCCTCTTCTCTGATTCTGGCCGGAAGAAAT
ACAGGCGAGCTGTTTTTTGGCGAGGGCTCTAGACTGACCGTGCTTGAGGAC
CTGAAGAACGTGTTCCCCCCAGAGGTGGCCGTGTTCGAGCCTAGCGAGGCC
GAGATCAGCCACACCCAGAAAGCCACCCTCGTGTGCCTGGCCACCGGCTTT
TACCCCGACCACGTGGAACTGTCTTGGTGGGTCAACGGCAAAGAGGTGCAC
AGCGGCGTCTGCACCGACCCCCAGCCCCTGAAAGAGCAGCCCGCCCTGAAC
GACAGCCGGTACTGTCTGAGCAGCAGACTGAGAGTGTCCGCCACCTTCTGG
CAGAACCCCCGGAACCACTTCAGATGCCAGGTGCAGTTCTACGGCCTGAGC
GAGAACGACGAGTGGACCCAGGACCGGGCCAAGCCCGTGACCCAGATCGT
GTCTGCTGAGGCCTGGGGCAGAGCCGATTGCGGCTTCACCAGCGAGAGCTA
CCAGCAGGGCGTGCTGAGCGCCACCATCCTGTACGAGATCCTGCTGGGCAA
GGCCACCCTGTACGCCGTGCTGGTGTCCGCCCTGGTGCTGATGGCCATGGTC
AAGCGGAAGGACAGCCGGGGCGGTTCCGGAGCCACGAACTTCTCTCTGTTA
AAGCAAGCAGGAGACGTGGAAGAAAACCCCGGTCCCATGAAGAAGCACCTG
ACCACGTTCCTGGTGATTCTGTGGCTGTACTTCTACAGGGGCAACGGCAAGAAT
CAGGTTGAGCAGTCTCCTCAGTCCCTGATTATTCTTGAGGGCAAGAATTGTA
CCCTGCAGTGCAACTACACAGTGAGCCCTTTCAGCAACCTGAGGTGGTACA
AGCAGGATACAGGAAGAGGACCTGTGAGTTTGACAATCATGACCTTCAGCG
AGAACACCAAGAGCAACGGCAGATATACAGCCACCCTGGATGCCGACACA
AAGCAGTCTTCTCTGCACATTACAGCCTCTCAGCTGAGCGATTCTGCCAGCT
ACATCTGCGTGGTGAGAGACAGCAATTACCAGCTGATTTGGGGAGCTGGCA
CAAAGCTGATCATCAAGCCCGACATCCAGAATCCCGATCCTGCTGTGTACC
AGCTGCGGGACAGCAAGAGCAGCGACAAGAGCGTGTGCCTGTTCACCGACT
TCGACAGCCAGACCAACGTGTCCCAGAGCAAGGACAGCGACGTGTACATCA
CCGATAAGTGCGTGCTGGACATGCGGAGCATGGACTTCAAGAGCAACAGCG
CCGTGGCCTGGTCCAACAAGAGCGACTTCGCCTGCGCCAACGCCTTCAACA
ACAGCATTATCCCCGAGGACACATTCTTCCCAAGCCCCGAGAGCAGCTGCG
ACGTGAAGCTGGTGGAAAAGAGCTTCGAGACAGACACCAACCTGAACTTCC
AGAACCTCAGCGTGATCGGCTTCCGGATCCTGCTGCTGAAGGTGGCCGGCT
TCAACCTGCTGATGACCCTGCGGCTGTGGTCCAGCTGA
(SOX2 TCR#02 TCRβ-P2A-TCRα, amino acid)
SEQ ID NO.: 24
MGIRLLCRVAFCFLAVGLVDVKVTQSSRYLVKRTGEKVFLECVQDMDHENMFW
YRQDPGLGLRLIYFSYDVKMKEKGDIPEGYSVSREKKERFSLILESASTNQTSM
YLCASSLILAGRNTGELFFGEGSRLTVLEDLKNVFPPEVAVFEPSEAEISHTQKA
TLVCLATGFYPDHVELSWWVNGKEVHSGVCTDPQPLKEQPALNDSRYCLSSRL
RVSATFWQNPRNHFRCQVQFYGLSENDEWTQDRAKPVTQIVSAEAWGRADCG
FTSESYQQGVLSATILYEILLGKATLYAVLVSALVLMAMVKRKDSRGGSGATN
FSLLKQAGDVEENPGPMKKHLTTFLVILWLYFYRGNGKNQVEQSPQSLIILEGKN
CTLQCNYTVSPFSNLRWYKQDTGRGPVSLTIMTFSENTKSNGRYTATLDADTK
QSSLHITASQLSDSASYICVVRDSNYQLIWGAGTKLIIKPDIQNPDPAVYQLRDS
KSSDKSVCLFTDFDSQTNVSQSKDSDVYITDKCVLDMRSMDFKSNSAVAWSN
KSDFACANAFNNSIIPEDTFFPSPESSCDVKLVEKSFETDTNLNFQNLSVIGFRILL
LKVAGFNLLMTLRLWSS
(SOX2 TCR#02 TCRVβ, amino acid)
SEQ ID NO.: 25
DVKVTQSSRYLVKRTGEKVFLECVQDMDHENMFWYRQDPGLGLRLIYFSYDV
KMKEKGDIPEGYSVSREKKERFSLILESASTNQTSMYLCASSLILAGRNTGELFF
GEGSRLTVLE
(SOX2 TCR#02 CDR1β, amino acid)
SEQ ID NO.: 26
MDHEN
(SOX2 TCR#02 CDR2β, amino acid)
SEQ ID NO.: 27
SYDVKM
(SOX2 TCR#02 CDR3β, amino acid)
SEQ ID NO.: 28
ASSLILAGRNTGELF
(SOX2 TCR#02 CDR3β-junction, amino acid)
SEQ ID NO.: 29
CASSLILAGRNTGELFF
(SOX2 TCR#02 TCRVα, amino acid)
SEQ ID NO.: 30
KNQVEQSPQSLIILEGKNCTLQCNYTVSPFSNLRWYKQDTGRGPVSLTIMTFSE
NTKSNGRYTATLDADTKQSSLHITASQLSDSASYICVVRDSNYQLIWGAGTKLII
KPD
(SOX2 TCR#02 CDR1α, amino acid)
SEQ ID NO.: 31
VSPFSN
(SOX2 TCR#02 CDR2α, amino acid)
SEQ ID NO.: 32
MTFSENT
(SOX2 TCR#02 CDR3α, amino acid)
SEQ ID NO.: 33
VVRDSNYQLI
(SOX2 TCR#02 CDR3α-junction, amino acid)
SEQ ID NO.: 34
CVVRDSNYQLIW
(SOX2 TCR#04 TCRβ-P2A-TCRα, codon-optimized nucleotide)
SEQ ID NO.: 35
ATGGGACCTGGACTGTTATGTTGGGTTCTGCTGTGTCTGCTTGGAGCTGGACCT
GTTGATGCTGGAGTTACACAGAGCCCTACACACCTGATCAAGACAAGAGGA
CAGCAGGTGACCCTGAGATGTAGCCCTATTTCTGGCCACAAGAGCGTGAGC
TGGTATCAGCAGGTTTTAGGACAGGGACCCCAGTTCATCTTCCAGTACTACG
AGAAGGAGGAGAGAGGCAGAGGCAATTTCCCTGACAGATTTAGCGCCCGG
CAGTTCCCCAATTATAGCTCTGAGCTGAACGTGAATGCCCTGCTGCTTGGCG
ATTCTGCTCTGTACCTGTGTGCCTCTTCTCTGTTTATCAGCCTGATGCTGGAT
ACCCAGTACTTCGGCCCTGGCACAAGACTGACAGTGCTGGAAGACCTGAAG
AACGTGTTCCCCCCAGAGGTGGCCGTGTTCGAGCCTAGCGAGGCCGAGATC
AGCCACACCCAGAAAGCCACCCTCGTGTGCCTGGCCACCGGCTTTTACCCC
GACCACGTGGAACTGTCTTGGTGGGTCAACGGCAAAGAGGTGCACAGCGGC
GTCTGCACCGACCCCCAGCCCCTGAAAGAGCAGCCCGCCCTGAACGACAGC
CGGTACTGTCTGAGCAGCAGACTGAGAGTGTCCGCCACCTTCTGGCAGAAC
CCCCGGAACCACTTCAGATGCCAGGTGCAGTTCTACGGCCTGAGCGAGAAC
GACGAGTGGACCCAGGACCGGGCCAAGCCCGTGACCCAGATCGTGTCTGCT
GAGGCCTGGGGCAGAGCCGATTGCGGCTTCACCAGCGAGAGCTACCAGCAG
GGCGTGCTGAGCGCCACCATCCTGTACGAGATCCTGCTGGGCAAGGCCACC
CTGTACGCCGTGCTGGTGTCCGCCCTGGTGCTGATGGCCATGGTCAAGCGG
AAGGACAGCCGGGGCGGTTCCGGAGCCACGAACTTCTCTCTGTTAAAGCAA
GCAGGAGACGTGGAAGAAAACCCCGGTCCCATGGTGAAGATCCGGCAGTTCC
TCCTGGCTATTCTGTGGCTGCAACTGTCTTGTGTGTCAGCAGCCAAGAATGAAG
TGGAGCAGTCCCCTCAGAACCTGACCGCACAGGAAGGCGAGTTTATCACCA
TCAACTGCAGCTACTCCGTAGGCATAAGCGCCCTGCATTGGCTCCAGCAGC
ACCCTGGAGGAGGAATTGTGTCTCTGTTTATGCTGAGCAGCGGAAAAAAAA
AGCACGGCCGGCTGATTGCCACCATCAACATCCAGGAGAAGCATAGCAGCC
TACACATTACAGCGTCTCATCCCAGGGATTCTGCCGTGTACATCTGTGCCAC
ACTGACACAAGGAGGAAGCGAAAAACTGGTGTTTGGCAAGGGCACAAAGC
TGACCGTGAATCCCTACATCCAGAATCCCGATCCTGCCGTGTACCAGCTGCG
GGACAGCAAGAGCAGCGACAAGAGCGTGTGCCTGTTCACCGACTTCGACAG
CCAGACCAACGTGTCCCAGAGCAAGGACAGCGACGTGTACATCACCGATAA
GTGCGTGCTGGACATGCGGAGCATGGACTTCAAGAGCAACAGCGCCGTGGC
CTGGTCCAACAAGAGCGACTTCGCCTGCGCCAACGCCTTCAACAACAGCAT
TATCCCCGAGGACACATTCTTCCCAAGCCCCGAGAGCAGCTGCGACGTGAA
GCTGGTGGAAAAGAGCTTCGAGACAGACACCAACCTGAACTTCCAGAACCT
CAGCGTGATCGGCTTCCGGATCCTGCTGCTGAAGGTGGCCGGCTTCAACCTG
CTGATGACCCTGCGGCTGTGGTCCAGCTGA
(SOX2 TCR#04 TCRβ-P2A-TCRα, amino acid)
SEQ ID NO.: 36
MGPGLLCWVLLCLLGAGPVDAGVTQSPTHLIKTRGQQVTLRCSPISGHKSVSWY
QQVLGQGPQFIFQYYEKEERGRGNFPDRFSARQFPNYSSELNVNALLLGDSALY
LCASSLFISLMLDTQYFGPGTRLTVLEDLKNVFPPEVAVFEPSEAEISHTQKATL
VCLATGFYPDHVELSWWVNGKEVHSGVCTDPQPLKEQPALNDSRYCLSSRLR
VSATFWQNPRNHFRCQVQFYGLSENDEWTQDRAKPVTQIVSAEAWGRADCGF
TSESYQQGVLSATILYEILLGKATLYAVLVSALVLMAMVKRKDSRGGSGATNF
SLLKQAGDVEENPGPMVKIRQFLLAILWLQLSCVSAAKNEVEQSPQNLTAQEGEFI
TINCSYSVGISALHWLQQHPGGGIVSLFMLSSGKKKHGRLIATINIQEKHSSLHIT
ASHPRDSAVYICATLTQGGSEKLVFGKGTKLTVNPYIQNPDPAVYQLRDSKSSD
KSVCLFTDFDSQTNVSQSKDSDVYITDKCVLDMRSMDFKSNSAVAWSNKSDF
ACANAFNNSIIPEDTFFPSPESSCDVKLVEKSFETDTNLNFQNLSVIGFRILLLKV
AGFNLLMTLRLWSS
(SOX2 TCR#04 TCRVβ, amino acid)
SEQ ID NO.: 37
DAGVTQSPTHLIKTRGQQVTLRCSPISGHKSVSWYQQVLGQGPQFIFQYYEKEE
RGRGNFPDRFSARQFPNYSSELNVNALLLGDSALYLCASSLFISLMLDTQYFGP
GTRLTVLE
(SOX2 TCR#04 CDR1β, amino acid)
SEQ ID NO.: 38
SGHKS
(SOX2 TCR#04 CDR2β, amino acid)
SEQ ID NO.: 39
YYEKEE
(SOX2 TCR#04 CDR3β, amino acid)
SEQ ID NO.: 40
ASSLFISLMLDTQY
(SOX2 TCR#04 CDR3β-junction, amino acid)
SEQ ID NO.: 41
CASSLFISLMLDTQYF
(SOX2 TCR#04 TCRVα, amino acid)
SEQ ID NO.: 42
KNEVEQSPQNLTAQEGEFITINCSYSVGISALHWLQQHPGGGIVSLFMLSSGKK
KHGRLIATINIQEKHSSLHITASHPRDSAVYICATLTQGGSEKLVFGKGTKLTVN
PY
(SOX2 TCR#04 CDR1α, amino acid)
SEQ ID NO.: 43
VGISA
(SOX2 TCR#04 CDR2α, amino acid)
SEQ ID NO.: 44
LSSGK
(SOX2 TCR#04 CDR3α, amino acid)
SEQ ID NO.: 45
ATLTQGGSEKLV
(SOX2 TCR#04 CDR3α-junction, amino acid)
SEQ ID NO.: 46
CATLTQGGSEKLVF
(SOX2 TCR#05 TCRβ-P2A-TCRα, codon-optimized nucleotide)
SEQ ID NO.: 47
ATGGGCATCAGACTCCTTTGCAGGGTGGCCTTTTGCTTTCTGGCTGTGGGATTAG
TGGACGTGAAGGTTACCCAGTCCAGCAGATACCTGGTTAAGAGAACCGGCG
AGAAGGTGTTCCTGGAATGTGTGCAGGACATGGATCACGAAAATATGTTTT
GGTACAGACAGGACCCTGGACTGGGACTGAGACTGATCTACTTCAGCTACG
ACGTGAAGATGAAGGAGAAGGGCGATATCCCTGAGGGCTACAGCGTGAGC
AGAGAGAAAAAGGAGAGGTTCAGCCTGATTCTGGAAAGCGCCAGCACAAA
TCAAACATCTATGTACCTTTGCGCCTCTAGACCTGAAATGGGCGGAAGAGG
AAATCAGCCTCAGCATTTTGGCGATGGCACAAGACTGAGCATCCTGGAGGA
TCTGAACAAGGTGTTCCCCCCAGAGGTGGCCGTGTTCGAGCCTTCTGAGGCC
GAGATCTCCCACACCCAGAAAGCCACCCTCGTGTGCCTGGCCACCGGCTTTT
TCCCCGACCACGTGGAACTGTCTTGGTGGGTCAACGGCAAAGAGGTGCACT
CCGGCGTGTGCACCGATCCCCAGCCTCTGAAAGAACAGCCCGCCCTGAACG
ACAGCCGGTACTGCCTGAGCAGCAGACTGAGAGTGTCCGCCACCTTCTGGC
AGAACCCCCGGAACCACTTCAGATGCCAGGTGCAGTTCTACGGCCTGAGCG
AGAACGACGAGTGGACCCAGGACAGAGCCAAGCCCGTGACACAGATCGTG
TCTGCCGAAGCCTGGGGCAGAGCCGATTGCGGCTTTACCTCCGTGTCCTATC
AGCAGGGCGTGCTGAGCGCCACAATCCTGTACGAGATCCTGCTGGGCAAGG
CCACCCTGTACGCCGTGCTGGTGTCTGCCCTGGTGCTGATGGCCATGGTCAA
GCGGAAGGACTTCGGTTCCGGAGCCACGAACTTCTCTCTGTTAAAGCAAGC
AGGAGACGTGGAAGAAAACCCCGGTCCCATGAGCCTGTCTTCTCTGCTGAAG
GTGGTGACTGCTAGCCTCTGGTTAGGACCTGGAATTGCCCAGAAGATCACCCA
GACACAACCTGGCATGTTTGTGCAGGAGAAAGAGGCTGTGACCCTGGATTG
CACCTACGATACCAGCGATCCTTCTTACGGCCTTTTCTGGTACAAACAGCCT
AGCTCCGGCGAGATGATCTTCCTGATCTACCAGGGCAGCTACGACCAGCAG
AACGCAACCGAGGGTAGATATAGCCTGAATTTTCAGAAGGCAAGAAAGTCA
GCCAACCTGGTGATCTCTGCCTCTCAACTGGGCGATTCTGCCATGTACTTTT
GTGCCATGAGAGAGGGCATGGGCTTCCAGAAACTGGTGTTTGGCACAGGCA
CAAGGCTGCTGGTTTCTCCCAATATCCAGAATCCTGACCCTGCCGTGTACCA
GCTGCGGGACAGCAAGAGCAGCGACAAGAGCGTGTGCCTGTTCACCGACTT
CGACAGCCAGACCAACGTGTCCCAGAGCAAGGACAGCGACGTGTACATCAC
CGATAAGTGCGTGCTGGACATGCGGAGCATGGACTTCAAGAGCAACAGCGC
CGTGGCCTGGTCCAACAAGAGCGACTTCGCCTGCGCCAACGCCTTCAACAA
CAGCATTATCCCCGAGGACACATTCTTCCCAAGCCCCGAGAGCAGCTGCGA
CGTGAAGCTGGTGGAAAAGAGCTTCGAGACAGACACCAACCTGAACTTCCA
GAACCTCAGCGTGATCGGCTTCCGGATCCTGCTGCTGAAGGTGGCCGGCTTC
AACCTGCTGATGACCCTGCGGCTGTGGTCCAGCTGA
(SOX2 TCR#05 TCRβ-P2A-TCRα, amino acid)
SEQ ID NO.: 48
MGIRLLCRVAFCFLAVGLVDVKVTQSSRYLVKRTGEKVFLECVQDMDHENMFW
YRQDPGLGLRLIYFSYDVKMKEKGDIPEGYSVSREKKERFSLILESASTNQTSM
YLCASRPEMGGRGNQPQHFGDGIRLSILEDLNKVFPPEVAVFEPSEAEISHTQK
ATLVCLATGFFPDHVELSWWVNGKEVHSGVCTDPQPLKEQPALNDSRYCLSSR
LRVSATFWQNPRNHFRCQVQFYGLSENDEWTQDRAKPVTQIVSAEAWGRADC
GFTSVSYQQGVLSATILYEILLGKATLYAVLVSALVLMAMVKRKDFGSGATNF
SLLKQAGDVEENPGPMSLSSLLKVVTASLWLGPGIAQKITQTQPGMFVQEKEAVT
LDCTYDTSDPSYGLFWYKQPSSGEMIFLIYQGSYDQQNATEGRYSLNFQKARK
SANLVISASQLGDSAMYFCAMREGMGFQKLVFGTGTRLLVSPNIQNPDPAVYQ
LRDSKSSDKSVCLFTDFDSQTNVSQSKDSDVYITDKCVLDMRSMDFKSNSAVA
WSNKSDFACANAFNNSIIPEDTFFPSPESSCDVKLVEKSFETDTNLNFQNLSVIGF
RILLLKVAGFNLLMTLRLWSS
(SOX2 TCR#05 TCR Vβ, amino acid)
SEQ ID NO.: 49
DVKVTQSSRYLVKRTGEKVFLECVQDMDHENMFWYRQDPGLGLRLIYFSYDV
KMKEKGDIPEGYSVSREKKERFSLILESASTNQTSMYLCASRPEMGGRGNQPQ
HFGDGTRLSILE
(SOX2 TCR#05 CDR1β, amino acid)
SEQ ID NO.: 50
MDHEN
(SOX2 TCR#05 CDR2β, amino acid)
SEQ ID NO.: 51
SYDVKM
(SOX2 TCR#05 CDR3β, amino acid)
SEQ ID NO.: 52
ASRPEMGGRGNQPQH
(SOX2 TCR#05 CDR3β-junction, amino acid)
SEQ ID NO.: 53
CASRPEMGGRGNQPQHF
(SOX2 TCR#05 TCR Vα, amino acid)
SEQ ID NO.: 54
AQKITQTQPGMFVQEKEAVTLDCTYDTSDPSYGLFWYKQPSSGEMIFLIYQGSY
DQQNATEGRYSLNFQKARKSANLVISASQLGDSAMYFCAMREGMGFQKLVFG
TGTRLLVSPN
(SOX2 TCR#05 CDR1α, amino acid)
SEQ ID NO.: 55
TSDPSYG
(SOX2 TCR#05 CDR2α, amino acid)
SEQ ID NO.: 56
QGSYDQQN
(SOX2 TCR#05 CDR3α, amino acid)
SEQ ID NO.: 57
AMREGMGFQKLV
(SOX2 TCR#05 CDR3α-junction, amino acid)
SEQ ID NO.: 58
CAMREGMGFQKLVF
(SOX2 TCR#06 TCRβ-P2A-TCRα, codon-optimized nucleotide)
SEQ ID NO.: 59
ATGCTTTTACTCCTTCTCTTGCTCGGACCTGCTGGATCTGGATTAGGAGCTGTTG
TGTCTCAGCACCCTTCTTGGGTGATCTGTAAAAGCGGCACAAGCGTGAAAA
TTGAGTGCAGGAGCCTGGATTTTCAGGCCACCACCATGTTCTGGTATAGGCA
GTTCCCCAAGCAGTCTCTGATGCTGATGGCCACCTCTAATGAGGGCTCTAAG
GCCACATATGAGCAGGGCGTAGAGAAGGACAAGTTCCTGATCAACCACGCC
TCTCTGACCCTGTCTACCCTGACAGTTACATCTGCCCACCCTGAGGATAGCA
GCTTTTACATCTGTAGCGCCAGAGGCAAGACAAGCCCTAGACCTGAAACCC
AGTACTTCGGACCTGGCACAAGACTGTTAGTTCTGGAGGACCTGAAGAACG
TGTTCCCCCCAGAGGTGGCCGTGTTCGAGCCTAGCGAGGCCGAGATCAGCC
ACACCCAGAAAGCCACCCTCGTGTGCCTGGCCACCGGCTTTTACCCCGACC
ACGTGGAACTGTCTTGGTGGGTCAACGGCAAAGAGGTGCACAGCGGCGTCT
GCACCGACCCCCAGCCCCTGAAAGAGCAGCCCGCCCTGAACGACAGCCGGT
ACTGTCTGAGCAGCAGACTGAGAGTGTCCGCCACCTTCTGGCAGAACCCCC
GGAACCACTTCAGATGCCAGGTGCAGTTCTACGGCCTGAGCGAGAACGACG
AGTGGACCCAGGACCGGGCCAAGCCCGTGACCCAGATCGTGTCTGCTGAGG
CCTGGGGCAGAGCCGATTGCGGCTTCACCAGCGAGAGCTACCAGCAGGGCG
TGCTGAGCGCCACCATCCTGTACGAGATCCTGCTGGGCAAGGCCACCCTGT
ACGCCGTGCTGGTGTCCGCCCTGGTGCTGATGGCCATGGTCAAGCGGAAGG
ACAGCCGGGGCGGTTCCGGAGCCACGAACTTCTCTCTGTTAAAGCAAGCAG
GAGACGTGGAAGAAAACCCCGGTCCCATGGAGTCTTCATTGGGTGGTGTGCT
GCTGATTCTGTGGCTGCAGGTAGATTGGGTCAAGAGCCAGAAGATCGAGCAGA
ATTCTGAAGCCCTGAACATCCAGGAGGGCAAGACAGCTACACTGACCTGCA
ACTACACCAACTACTCTCCTGCTTACCTGCAGTGGTACAGACAGGATCCTGG
CAGAGGACCTGTGTTTCTGCTGCTGATTAGAGAAAACGAAAAGGAGAAAAG
AAAAGAGAGACTGAAGGTTACCTTCGACACAACCCTGAAGCAGAGCCTGTT
TCACATTACAGCCAGCCAACCCGCCGATTCTGCCACATACCTGTGTGCTCTG
GGACACAAGCTGACATTTGGAGGAGGAACAAGAGTTCTGGTGAAGCCCAA
CATCCAGAATCCCGATCCTGCCGTGTACCAGCTGCGGGACAGCAAGAGCAG
CGACAAGAGCGTGTGCCTGTTCACCGACTTCGACAGCCAGACCAACGTGTC
CCAGAGCAAGGACAGCGACGTGTACATCACCGATAAGTGCGTGCTGGACAT
GCGGAGCATGGACTTCAAGAGCAACAGCGCCGTGGCCTGGTCCAACAAGA
GCGACTTCGCCTGCGCCAACGCCTTCAACAACAGCATTATCCCCGAGGACA
CATTCTTCCCAAGCCCCGAGAGCAGCTGCGACGTGAAGCTGGTGGAAAAGA
GCTTCGAGACAGACACCAACCTGAACTTCCAGAACCTCAGCGTGATCGGCT
TCCGGATCCTGCTGCTGAAGGTGGCCGGCTTCAACCTGCTGATGACCCTGCG
GCTGTGGTCCAGCTGA
(SOX2 TCR#06 TCRβ-P2A-TCRα, amino acid)
SEQ ID NO.: 60
MLLLLLLLGPAGSGLGAVVSQHPSWVICKSGTSVKIECRSLDFQATTMFWYRQF
PKQSLMLMATSNEGSKATYEQGVEKDKFLINHASLTLSTLTVTSAHPEDSSFYI
CSARGKTSPRPETQYFGPGTRLLVLEDLKNVFPPEVAVFEPSEAEISHTQKATLV
CLATGFYPDHVELSWWVNGKEVHSGVCTDPQPLKEQPALNDSRYCLSSRLRVS
ATFWQNPRNHFRCQVQFYGLSENDEWTQDRAKPVTQIVSAEAWGRADCGFTS
ESYQQGVLSATILYEILLGKATLYAVLVSALVLMAMVKRKDSRGGSGATNFSL
LKQAGDVEENPGPMESSLGGVLLILWLQVDWVKSQKIEQNSEALNIQEGKTATLT
CNYTNYSPAYLQWYRQDPGRGPVFLLLIRENEKEKRKERLKVTFDTTLKQSLF
HITASQPADSATYLCALGHKLTFGGGTRVLVKPNIQNPDPAVYQLRDSKSSDKS
VCLFTDFDSQTNVSQSKDSDVYITDKCVLDMRSMDFKSNSAVAWSNKSDFAC
ANAFNNSIIPEDTFFPSPESSCDVKLVEKSFETDTNLNFQNLSVIGFRILLLKVAG
FNLLMTLRLWSS
(SOX2 TCR#06 TCRVβ, amino acid)
SEQ ID NO.: 61
GAVVSQHPSWVICKSGTSVKIECRSLDFQATTMFWYRQFPKQSLMLMATSNEG
SKATYEQGVEKDKFLINHASLTLSTLTVTSAHPEDSSFYICSARGKTSPRPETQY
FGPGTRLLVLE
(SOX2 TCR#06 CDR1β, amino acid)
SEQ ID NO.: 62
DFQATT
(SOX2 TCR#06 CDR2β, amino acid)
SEQ ID NO.: 63
SNEGSKA
(SOX2 TCR#06 CDR3β, amino acid)
SEQ ID NO.: 64
SARGKTSPRPETQY
(SOX2 TCR#06 CDR3β-junction, amino acid)
SEQ ID NO.: 65
CSARGKTSPRPETQYF
(SOX2 TCR#06 TCRVα, amino acid)
SEQ ID NO.: 66
SQKIEQNSEALNIQEGKTATLTCNYTNYSPAYLQWYRQDPGRGPVFLLLIRENE
KEKRKERLKVTFDTTLKQSLFHITASQPADSATYLCALGHKLTFGGGTRVLVKP
N
(SOX2 TCR#06 CDR1α, amino acid)
SEQ ID NO.: 67
NYSPAY
(SOX2 TCR#06 CDR2α, amino acid)
SEQ ID NO.: 68
IRENEKE
(SOX2 TCR#06 CDR3α, amino acid)
SEQ ID NO.: 69
ALGHKLT
(SOX2 TCR#06 CDR3α-junction, amino acid)
SEQ ID NO.: 70
CALGHKLTF
(SOX2 TCR#07 TCRβ-P2A-TCRα, codon-optimized nucleotide)
SEQ ID NO.: 71
ATGGGCACAAGACTCCTTTTCTGGGTCGCCTTTTGTCTGCTTGGAGCCGATCATA
CCGGCGCCGGCGTGAGCCAGTCTCCCAGCAATAAGGTCACCGAGAAGGGCA
AAGATGTGGAGCTGAGATGCGACCCTATTTCTGGCCATACCGCCCTGTACTG
GTACAGACAATCTCTGGGACAGGGGCTAGAGTTCCTGATCTACTTTCAGGG
CAATAGCGCCCCCGACAAATCTGGACTTCCTAGCGACAGGTTTTCTGCCGA
GAGAACAGGAGGATCTGTTTCTACCCTGACCATCCAGAGAACACAGCAGGA
GGATTCTGCCGTGTACCTGTGTGCCAGCAGAAGAACAGGATGGAGCTACAA
TGAGCAGTTCTTTGGCCCTGGAACCAGACTGACAGTGCTGGAGGACCTGAA
GAACGTGTTCCCCCCAGAGGTGGCCGTGTTCGAGCCTAGCGAGGCCGAGAT
CAGCCACACCCAGAAAGCCACCCTCGTGTGCCTGGCCACCGGCTTTTACCCC
GACCACGTGGAACTGTCTTGGTGGGTCAACGGCAAAGAGGTGCACAGCGGC
GTCTGCACCGACCCCCAGCCCCTGAAAGAGCAGCCCGCCCTGAACGACAGC
CGGTACTGTCTGAGCAGCAGACTGAGAGTGTCCGCCACCTTCTGGCAGAAC
CCCCGGAACCACTTCAGATGCCAGGTGCAGTTCTACGGCCTGAGCGAGAAC
GACGAGTGGACCCAGGACCGGGCCAAGCCCGTGACCCAGATCGTGTCTGCT
GAGGCCTGGGGCAGAGCCGATTGCGGCTTCACCAGCGAGAGCTACCAGCAG
GGCGTGCTGAGCGCCACCATCCTGTACGAGATCCTGCTGGGCAAGGCCACC
CTGTACGCCGTGCTGGTGTCCGCCCTGGTGCTGATGGCCATGGTCAAGCGG
AAGGACAGCCGGGGCGGTTCCGGAGCCACGAACTTCTCTCTGTTAAAGCAA
GCAGGAGACGTGGAAGAAAACCCCGGTCCCATGATCAGCCTGAGAGTCCTGC
TCGTGATTCTGTGGCTGCAACTGTCTTGGGTTTGGAGCCAGAGAAAAGAAGTG
GAGCAGGATCCTGGCCCCTTTAATGTGCCTGAAGGAGCTACAGTGGCCTTC
AACTGCACATACAGCAACAGCGCCTCACAGAGCTTCTTCTGGTACAGACAG
GACTGCAGGAAGGAGCCTAAGCTGCTGATGTCTGTGTATAGCAGCGGCAAT
GAGGATGGCAGATTTACAGCCCAGCTGAACCGGGCTTCTCAGTACATCTCT
CTGCTGATCAGGGATAGCAAGCTGAGCGATTCTGCTACATACCTCTGCGTG
GTGTACGTGGAGTCTGGAGGCAGCTATATCCCTACATTCGGCAGAGGAACA
AGCCTGATTGTGCACCCCTATATCCAGAATCCCGACCCTGCTGTGTACCAGC
TGCGGGACAGCAAGAGCAGCGACAAGAGCGTGTGCCTGTTCACCGACTTCG
ACAGCCAGACCAACGTGTCCCAGAGCAAGGACAGCGACGTGTACATCACCG
ATAAGTGCGTGCTGGACATGCGGAGCATGGACTTCAAGAGCAACAGCGCCG
TGGCCTGGTCCAACAAGAGCGACTTCGCCTGCGCCAACGCCTTCAACAACA
GCATTATCCCCGAGGACACATTCTTCCCAAGCCCCGAGAGCAGCTGCGACG
TGAAGCTGGTGGAAAAGAGCTTCGAGACAGACACCAACCTGAACTTCCAGA
ACCTCAGCGTGATCGGCTTCCGGATCCTGCTGCTGAAGGTGGCCGGCTTCAA
CCTGCTGATGACCCTGCGGCTGTGGTCCAGCTGA
(SOX2 TCR#07 TCRβ-P2A-TCRα, amino acid)
SEQ ID NO.: 72
MGTRLLFWVAFCLLGADHTGAGVSQSPSNKVTEKGKDVELRCDPISGHTALYW
YRQSLGQGLEFLIYFQGNSAPDKSGLPSDRFSAERTGGSVSTLTIQRTQQEDSAV
YLCASRRTGWSYNEQFFGPGTRLTVLEDLKNVFPPEVAVFEPSEAEISHTQKAT
LVCLATGFYPDHVELSWWVNGKEVHSGVCTDPQPLKEQPALNDSRYCLSSRL
RVSATFWQNPRNHFRCQVQFYGLSENDEWTQDRAKPVTQIVSAEAWGRADCG
FTSESYQQGVLSATILYEILLGKATLYAVLVSALVLMAMVKRKDSRGGSGATN
FSLLKQAGDVEENPGPMISLRVLLVILWLQLSWVWSQRKEVEQDPGPFNVPEGAT
VAFNCTYSNSASQSFFWYRQDCRKEPKLLMSVYSSGNEDGRFTAQLNRASQYI
SLLIRDSKLSDSATYLCVVYVESGGSYIPTFGRGTSLIVHPYIQNPDPAVYQLRD
SKSSDKSVCLFTDFDSQTNVSQSKDSDVYITDKCVLDMRSMDFKSNSAVAWSN
KSDFACANAFNNSIIPEDTFFPSPESSCDVKLVEKSFETDTNLNFQNLSVIGFRILL
LKVAGFNLLMTLRLWSS
(SOX2 TCR#07 TCRVβ, amino acid)
SEQ ID NO.: 73
GAGVSQSPSNKVTEKGKDVELRCDPISGHTALYWYRQSLGQGLEFLIYFQGNS
APDKSGLPSDRFSAERTGGSVSTLTIQRTQQEDSAVYLCASRRTGWSYNEQFFG
PGTRLTVLE
(SOX2 TCR#07 CDR1β, amino acid)
SEQ ID NO.: 74
SGHTA
(SOX2 TCR#07 CDR2β, amino acid)
SEQ ID NO.: 75
FQGNSA
(SOX2 TCR#07 CDR3β, amino acid)
SEQ ID NO.: 76
ASRRTGWSYNEQF
(SOX2 TCR#07 CDR3β-junction, amino acid)
SEQ ID NO.: 77
CASRRTGWSYNEQFF
(SOX2 TCR#07 TCRVα, amino acid)
SEQ ID NO.: 78
RKEVEQDPGPFNVPEGATVAFNCTYSNSASQSFFWYRQDCRKEPKLLMSVYSS
GNEDGRFTAQLNRASQYISLLIRDSKLSDSATYLCVVYVESGGSYIPTFGRGTSL
IVHPY
(SOX2 TCR#07 CDR1α, amino acid)
SEQ ID NO.: 79
NSASQS
(SOX2 TCR#07 CDR2α, amino acid)
SEQ ID NO.: 80
VYSSGN
(SOX2 TCR#07 CDR3α, amino acid)
SEQ ID NO.: 81
VVYVESGGSYIPT
(SOX2 TCR#07 CDR3α-junction, amino acid)
SEQ ID NO.: 82
CVVYVESGGSYIPTF
(SOX2 TCR#09 TCRβ-P2A-TCRα, codon-optimized nucleotide)
SEQ ID NO.: 83
ATGGATACCTGGCTCGTCTGTTGGGCCATCTTTTCTCTGCTGAAAGCTGGCCTGA
CAGAACCTGAGGTTACACAGACACCTAGCCACCAGGTGACTCAGATGGGAC
AGGAAGTGATCCTGAGATGTGTGCCCATCAGCAATCACCTGTACTTTTATTG
GTACAGGCAGATTCTGGGCCAGAAGGTCGAATTCCTGGTGAGCTTTTACAA
TAACGAGATTAGCGAGAAATCCGAAATCTTCGACGATCAGTTCTCAGTGGA
GAGACCTGATGGCAGCAATTTTACCCTGAAGATTCGCAGCACAAAGCTGGA
GGATAGCGCCATGTACTTTTGTGCGAGTTCTAGACGAGGCGGGCCTATTGA
AACCCAGTATTTTGGCCCTGGAACAAGACTGCTGGTCCTTGAGGACCTGAA
GAACGTGTTCCCCCCAGAGGTGGCCGTGTTCGAGCCTAGCGAGGCCGAGAT
CAGCCACACCCAGAAAGCCACCCTCGTGTGCCTGGCCACCGGCTTTTACCCC
GACCACGTGGAACTGTCTTGGTGGGTCAACGGCAAAGAGGTGCACAGCGGC
GTCTGCACCGACCCCCAGCCCCTGAAAGAGCAGCCCGCCCTGAACGACAGC
CGGTACTGTCTGAGCAGCAGACTGAGAGTGTCCGCCACCTTCTGGCAGAAC
CCCCGGAACCACTTCAGATGCCAGGTGCAGTTCTACGGCCTGAGCGAGAAC
GACGAGTGGACCCAGGACCGGGCCAAGCCCGTGACCCAGATCGTGTCTGCT
GAGGCCTGGGGCAGAGCCGATTGCGGCTTCACCAGCGAGAGCTACCAGCAG
GGCGTGCTGAGCGCCACCATCCTGTACGAGATCCTGCTGGGCAAGGCCACC
CTGTACGCCGTGCTGGTGTCCGCCCTGGTGCTGATGGCCATGGTCAAGCGG
AAGGACAGCCGGGGCGGTTCCGGAGCCACGAACTTCTCTCTGTTAAAGCAA
GCAGGAGACGTGGAAGAAAACCCCGGTCCCATGATGAAGAGCCTGCGAGTCC
TCCTGGTGATTCTGTGGCTGCAACTTTCTTGGGTGTGGTCTCAGCAGAAAGAAG
TGGAGCAGGATCCTGGACCTCTGTCTGTGCCTGAAGGAGCTATTGTGAGCCT
CAATTGTACTTACAGCAATAGCGCCTTCCAGTACTTCATGTGGTACCGGCAG
TACAGCAGGAAGGGCCCTGAACTGCTCATGTATACCTATTCTAGCGGCAAT
AAGGAGGATGGCAGGTTTACAGCCCAGGTGGACAAGAGCTCCAAGTATATA
AGTTTATTCATCAGAGATAGCCAGCCTAGCGATTCTGCCACCTACCTGTGTG
CCATGTCTGCCGACAATGCAGGTAATATGCTGACATTTGGAGGCGGAACAA
GGCTGATGGTGAAACCCCACATCCAGAACCCCGACCCCGCTGTGTACCAGC
TGCGGGACAGCAAGAGCAGCGACAAGAGCGTGTGCCTGTTCACCGACTTCG
ACAGCCAGACCAACGTGTCCCAGAGCAAGGACAGCGACGTGTACATCACCG
ATAAGTGCGTGCTGGACATGCGGAGCATGGACTTCAAGAGCAACAGCGCCG
TGGCCTGGTCCAACAAGAGCGACTTCGCCTGCGCCAACGCCTTCAACAACA
GCATTATCCCCGAGGACACATTCTTCCCAAGCCCCGAGAGCAGCTGCGACG
TGAAGCTGGTGGAAAAGAGCTTCGAGACAGACACCAACCTGAACTTCCAGA
ACCTCAGCGTGATCGGCTTCCGGATCCTGCTGCTGAAGGTGGCCGGCTTCAA
CCTGCTGATGACCCTGCGGCTGTGGTCCAGCTGA
(SOX2 TCR#09 TCRβ-P2A-TCRα, amino acid)
SEQ ID NO.: 84
MDTWLVCWAIFSLLKAGLTEPEVTQTPSHQVTQMGQEVILRCVPISNHLYFYWY
RQILGQKVEFLVSFYNNEISEKSEIFDDQFSVERPDGSNFTLKIRSTKLEDSAMYF
CASSRRGGPIETQYFGPGTRLLVLEDLKNVFPPEVAVFEPSEAEISHTQKATLVC
LATGFYPDHVELSWWVNGKEVHSGVCTDPQPLKEQPALNDSRYCLSSRLRVS
ATFWQNPRNHFRCQVQFYGLSENDEWTQDRAKPVTQIVSAEAWGRADCGFTS
ESYQQGVLSATILYEILLGKATLYAVLVSALVLMAMVKRKDSRGGSGATNFSL
LKQAGDVEENPGPMMKSLRVLLVILWLQLSWVWSQQKEVEQDPGPLSVPEGAIV
SLNCTYSNSAFQYFMWYRQYSRKGPELLMYTYSSGNKEDGRFTAQVDKSSKYI
SLFIRDSQPSDSATYLCAMSADNAGNMLTFGGGTRLMVKPHIQNPDPAVYQLR
DSKSSDKSVCLFTDFDSQTNVSQSKDSDVYITDKCVLDMRSMDFKSNSAVAWS
NKSDFACANAFNNSIIPEDTFFPSPESSCDVKLVEKSFETDTNLNFQNLSVIGFRI
LLLKVAGFNLLMTLRLWSS
(SOX2 TCR#09 TCRVβ, amino acid)
SEQ ID NO.: 85
EPEVTQTPSHQVTQMGQEVILRCVPISNHLYFYWYRQILGQKVEFLVSFYNNEI
SEKSEIFDDQFSVERPDGSNFTLKIRSTKLEDSAMYFCASSRRGGPIETQYFGPGT
RLLVLE
(SOX2 TCR#09 CDR1β, amino acid)
SEQ ID NO.: 86
SNHLY
(SOX2 TCR#09 CDR2β, amino acid)
SEQ ID NO.: 87
FYNNEI
(SOX2 TCR#09 CR3β, amino acid)
SEQ ID NO.: 88
ASSRRGGPIETQY
(SOX2 TCR#09 CR3β-junction, amino acid)
SEQ ID NO.: 89
CASSRRGGPIETQYF
(SOX2 TCR#09 TCRVα, amino acid)
SEQ ID NO.: 90
QKEVEQDPGPLSVPEGAIVSLNCTYSNSAFQYFMWYRQYSRKGPELLMYTYSS
GNKEDGRFTAQVDKSSKYISLFIRDSQPSDSATYLCAMSADNAGNMLTFGGGT
RLMVKPH
(SOX2 TCR#09 CDR1α, amino acid)
SEQ ID NO.: 91
NSAFQY
(SOX2 TCR#09 CDR2α, amino acid)
SEQ ID NO.: 92
TYSSGN
(SOX2 TCR#09 CDR3α, amino acid)
SEQ ID NO.: 93
AMSADNAGNMLT
(SOX2 TCR#09 CDR3α-junction, amino acid)
SEQ ID NO.: 94
CAMSADNAGNMLTF
(SOX2 TCR#10 TCRβ-P2A-TCRα, codon-optimized nucleotide)
SEQ ID NO.: 95
ATGACCATTCGACTGCTGTGCTACATGGGCTTCTACTTCCTGGGAGCTGGACTGA
TGGAAGCCGACATCTACCAAACACCCAGATACCTGGTGATCGGAACAGGCA
AGAAGATCACCCTGGAGTGTAGCCAGACAATGGGACACGACAAGATGTACT
GGTACCAGCAGGATCCTGGAATGGAACTGCACCTGATCCACTACAGCTATG
GCGTGAACAGCACAGAGAAAGGCGATCTGAGCAGCGAGTCTACAGTGAGC
AGAATCAGAACCGAGCACTTCCCTCTGACACTGGAATCTGCTAGACCTAGC
CACACAAGCCAGTACCTGTGTGCTAGCTCTAGAGGACCTGAGGCCTTTTTTG
GACAGGGAACAAGACTGACAGTGGTGGAGGATCTGAACAAGGTGTTCCCCC
CAGAGGTGGCCGTGTTCGAGCCTTCTGAGGCCGAGATCTCCCACACCCAGA
AAGCCACCCTCGTGTGCCTGGCCACCGGCTTTTTCCCCGACCACGTGGAACT
GTCTTGGTGGGTCAACGGCAAAGAGGTGCACTCCGGCGTGTGCACCGATCC
CCAGCCTCTGAAAGAACAGCCCGCCCTGAACGACAGCCGGTACTGCCTGAG
CAGCAGACTGAGAGTGTCCGCCACCTTCTGGCAGAACCCCCGGAACCACTT
CAGATGCCAGGTGCAGTTCTACGGCCTGAGCGAGAACGACGAGTGGACCCA
GGACAGAGCCAAGCCCGTGACACAGATCGTGTCTGCCGAAGCCTGGGGCAG
AGCCGATTGCGGCTTTACCTCCGTGTCCTATCAGCAGGGCGTGCTGAGCGCC
ACAATCCTGTACGAGATCCTGCTGGGCAAGGCCACCCTGTACGCCGTGCTG
GTGTCTGCCCTGGTGCTGATGGCCATGGTCAAGCGGAAGGACTTCGGTTCC
GGAGCCACGAACTTCTCTCTGTTAAAGCAAGCAGGAGACGTGGAAGAAAAC
CCCGGTCCCATGAAGACCTTTGCCGGATTCTCATTCCTATTTCTCTGGCTGCAGC
TGGATTGTATGAGCAGAGGCGAAGATGTGGAACAGAGCCTGTTTCTGAGCGT
GAGAGAGGGCGATAGCTCTGTGATCAATTGCACATACACGGATAGCAGCAG
CACCTACCTGTACTGGTATAAACAGGAGCCGGGCGCGGGCCTGCAACTGCT
GACATACATCTTCAGCAATATGGATATGAAGCAGGACCAGAGGTTGACCGT
CTTACTCAATAAGAAAGACAAGCACCTGTCTCTGAGAATCGCTGACACTCA
GACTGGCGATAGCGCCATCTACTTCTGTGCTGGACCTCTTGGCGGATCTCAG
GGAAATCTTATCTTCGGTAAGGGCACAAAGCTGAGCGTGAAGCCCAATATA
CAAAACCCAGACCCCGCCGTGTACCAGCTGCGGGACAGCAAGAGCAGCGA
CAAGAGCGTGTGCCTGTTCACCGACTTCGACAGCCAGACCAACGTGTCCCA
GAGCAAGGACAGCGACGTGTACATCACCGATAAGTGCGTGCTGGACATGCG
GAGCATGGACTTCAAGAGCAACAGCGCCGTGGCCTGGTCCAACAAGAGCG
ACTTCGCCTGCGCCAACGCCTTCAACAACAGCATTATCCCCGAGGACACATT
CTTCCCAAGCCCCGAGAGCAGCTGCGACGTGAAGCTGGTGGAAAAGAGCTT
CGAGACAGACACCAACCTGAACTTCCAGAACCTCAGCGTGATCGGCTTCCG
GATCCTGCTGCTGAAGGTGGCCGGCTTCAACCTGCTGATGACCCTGCGGCTG
TGGTCCAGCTGA
(SOX2 TCR#10 TCRβ-P2A-TCRα, amino acid)
SEQ ID NO.: 96
MTIRLLCYMGFYFLGAGLMEADIYQTPRYLVIGTGKKITLECSQTMGHDKMYWY
QQDPGMELHLIHYSYGVNSTEKGDLSSESTVSRIRTEHFPLTLESARPSHTSQYL
CASSRGPEAFFGQGTRLTVVEDLNKVFPPEVAVFEPSEAEISHTQKATLVCLAT
GFFPDHVELSWWVNGKEVHSGVCTDPQPLKEQPALNDSRYCLSSRLRVSATF
WQNPRNHFRCQVQFYGLSENDEWTQDRAKPVTQIVSAEAWGRADCGFTSVSY
QQGVLSATILYEILLGKATLYAVLVSALVLMAMVKRKDFGSGATNFSLLKQAG
DVEENPGPMKTFAGFSFLFLWLQLDCMSRGEDVEQSLFLSVREGDSSVINCTYTD
SSSTYLYWYKQEPGAGLQLLTYIFSNMDMKQDQRLTVLLNKKDKHLSLRIADT
QTGDSAIYFCAGPLGGSQGNLIFGKGTKLSVKPNIQNPDPAVYQLRDSKSSDKS
VCLFTDFDSQTNVSQSKDSDVYITDKCVLDMRSMDFKSNSAVAWSNKSDFAC
ANAFNNSIIPEDTFFPSPESSCDVKLVEKSFETDTNLNFQNLSVIGFRILLLKVAG
FNLLMTLRLWSS
(SOX2 TCR#10 TCRVβ, amino acid)
SEQ ID NO.: 97
EADIYQTPRYLVIGTGKKITLECSQTMGHDKMYWYQQDPGMELHLIHYSYGV
NSTEKGDLSSESTVSRIRTEHFPLTLESARPSHTSQYLCASSRGPEAFFGQGTRLT
VVE
(SOX2 TCR#10 CDR1β, amino acid)
SEQ ID NO.: 98
MGHDK
(SOX2 TCR#10 CDR2β, amino acid)
SEQ ID NO.: 99
SYGVNS
(SOX2 TCR#10 CR3β, amino acid)
SEQ ID NO.: 100
ASSRGPEAF
(SOX2 TCR#10 CR3β-junction, amino acid)
SEQ ID NO.: 101
CASSRGPEAFF
(SOX2 TCR#10 TCRVα, amino acid)
SEQ ID NO.: 102
GEDVEQSLFLSVREGDSSVINCTYTDSSSTYLYWYKQEPGAGLQLLTYIFSNMD
MKQDQRLTVLLNKKDKHLSLRIADTQTGDSAIYFCAGPLGGSQGNLIFGKGTK
LSVKPN
(SOX2 TCR#10 CDR1α, amino acid)
SEQ ID NO.: 103
DSSSTY
(SOX2 TCR#10 CDR2α, amino acid)
SEQ ID NO.: 104
IFSNMDM
(SOX2 TCR#10 CDR3α, amino acid)
SEQ ID NO.: 105
AGPLGGSQGNLI
(SOX2 TCR#10 CDR3α-junction, amino acid)
SEQ ID NO.: 106
CAGPLGGSQGNLIF
(SOX2 TCR#11 TCRβ-P2A-TCRα, codon-optimized nucleotide)
SEQ ID NO.: 107
ATGGGACCTCAGCTTCTTGGATACGTTGTGCTGTGTCTGCTTGGAGCCGGACCTT
TAGAGGCTCAGGTTACCCAGAACCCCAGATACCTGATTACAGTGACAGGCA
AGAAGCTGACCGTGACCTGTAGCCAGAACATGAACCACGAGTACATGAGCT
GGTACAGACAGGATCCTGGACTGGGACTCCGGCAAATTTACTACAGCATGA
ACGTGGAGGTGACCGATAAAGGAGATGTGCCCGAAGGCTACAAGGTGAGC
AGGAAGGAGAAGAGGAATTTCCCCCTGATCCTGGAATCTCCTAGCCCTAAT
CAGACCAGCCTGTACTTCTGTGCCAGCAGCCCAAGGTCTCCCGAAGGAGGA
AGAGCCTACAATGAGCAGTTCTTTGGCCCTGGAACAAGACTGACCGTGCTG
GAAGACCTGAAGAACGTGTTCCCCCCAGAGGTGGCCGTGTTCGAGCCTAGC
GAGGCCGAGATCAGCCACACCCAGAAAGCCACCCTCGTGTGCCTGGCCACC
GGCTTTTACCCCGACCACGTGGAACTGTCTTGGTGGGTCAACGGCAAAGAG
GTGCACAGCGGCGTCTGCACCGACCCCCAGCCCCTGAAAGAGCAGCCCGCC
CTGAACGACAGCCGGTACTGTCTGAGCAGCAGACTGAGAGTGTCCGCCACC
TTCTGGCAGAACCCCCGGAACCACTTCAGATGCCAGGTGCAGTTCTACGGC
CTGAGCGAGAACGACGAGTGGACCCAGGACCGGGCCAAGCCCGTGACCCA
GATCGTGTCTGCTGAGGCCTGGGGCAGAGCCGATTGCGGCTTCACCAGCGA
GAGCTACCAGCAGGGCGTGCTGAGCGCCACCATCCTGTACGAGATCCTGCT
GGGCAAGGCCACCCTGTACGCCGTGCTGGTGTCCGCCCTGGTGCTGATGGC
CATGGTCAAGCGGAAGGACAGCCGGGGCGGTTCCGGAGCCACGAACTTCTC
TCTGTTAAAGCAAGCAGGAGACGTGGAAGAAAACCCCGGTCCCATGGAGAA
GATGCTGGAGTGTGCGTTCATCGTTCTGTGGCTGCAACTTGGATGGCTGTCTGG
AGAGGATCAGGTTACACAGTCTCCTGAAGCCCTGAGACTGCAAGAAGGAGA
GAGCTCATCGTTGAACTGCAGCTACACAGTGTCTGGACTGAGAGGCCTGTT
CTGGTATAGGCAGGATCCGGGCAAAGGACCTGAGTTCCTGTTTACCCTCTAT
TCAGCTGGGGAGGAGAAGGAGAAGGAGAGACTGAAGGCTACACTGACCAA
GAAGGAGAGCTTTCTTCACATTACTGCCCCCAAACCTGAGGATTCTGCCACA
TACCTGTGTGCTGCTCTGGACAGAGACAATTATGGCCAAAATTTCGTGTTTG
GCCCTGGAACCAGACTGTCTGTGCTGCCTTACATTCAGAACCCCGATCCTGC
CGTGTACCAGCTGCGGGACAGCAAGAGCAGCGACAAGAGCGTGTGCCTGTT
CACCGACTTCGACAGCCAGACCAACGTGTCCCAGAGCAAGGACAGCGACGT
GTACATCACCGATAAGTGCGTGCTGGACATGCGGAGCATGGACTTCAAGAG
CAACAGCGCCGTGGCCTGGTCCAACAAGAGCGACTTCGCCTGCGCCAACGC
CTTCAACAACAGCATTATCCCCGAGGACACATTCTTCCCAAGCCCCGAGAG
CAGCTGCGACGTGAAGCTGGTGGAAAAGAGCTTCGAGACAGACACCAACCT
GAACTTCCAGAACCTCAGCGTGATCGGCTTCCGGATCCTGCTGCTGAAGGT
GGCCGGCTTCAACCTGCTGATGACCCTGCGGCTGTGGTCCAGCTGA
(SOX2 TCR#11 TCRβ-P2A-TCRα, amino acid)
SEQ ID NO.: 108
MGPQLLGYVVLCLLGAGPLEAQVTQNPRYLITVTGKKLTVTCSQNMNHEYMSW
YRQDPGLGLRQIYYSMNVEVTDKGDVPEGYKVSRKEKRNFPLILESPSPNQTSL
YFCASSPRSPEGGRAYNEQFFGPGTRLTVLEDLKNVFPPEVAVFEPSEAEISHTQ
KATLVCLATGFYPDHVELSWWVNGKEVHSGVCTDPQPLKEQPALNDSRYCLS
SRLRVSATFWQNPRNHFRCQVQFYGLSENDEWTQDRAKPVTQIVSAEAWGRA
DCGFTSESYQQGVLSATILYEILLGKATLYAVLVSALVLMAMVKRKDSRGGSG
ATNFSLLKQAGDVEENPGPMEKMLECAFIVLWLQLGWLSGEDQVTQSPEALRLQ
EGESSSLNCSYTVSGLRGLFWYRQDPGKGPEFLFTLYSAGEEKEKERLKATLTK
KESFLHITAPKPEDSATYLCAALDRDNYGQNFVFGPGTRLSVLPYIQNPDPAVY
QLRDSKSSDKSVCLFTDFDSQTNVSQSKDSDVYITDKCVLDMRSMDFKSNSAV
AWSNKSDFACANAFNNSIIPEDTFFPSPESSCDVKLVEKSFETDTNLNFQNLSVI
GFRILLLKVAGFNLLMTLRLWSS
(SOX2 TCR#11 TCRVβ, amino acid)
SEQ ID NO.: 109
EAQVTQNPRYLITVTGKKLTVTCSQNMNHEYMSWYRQDPGLGLRQIYYSMNV
EVTDKGDVPEGYKVSRKEKRNFPLILESPSPNQTSLYFCASSPRSPEGGRAYNEQ
FFGPGTRLTVLE
(SOX2 TCR#11 CDR1β, amino acid)
SEQ ID NO.: 110
MNHEY
(SOX2 TCR#11 CDR2β, amino acid)
SEQ ID NO.: 111
SMNVEV
(SOX2 TCR#11 CDR3β, amino acid)
SEQ ID NO.: 112
ASSPRSPEGGRAYNEQF
(SOX2 TCR#11 CDR3β-junction, amino acid)
SEQ ID NO.: 113
CASSPRSPEGGRAYNEQFF
(SOX2 TCR#11 TCRVα, amino acid)
SEQ ID NO.: 114
EDQVTQSPEALRLQEGESSSLNCSYTVSGLRGLFWYRQDPGKGPEFLFTLYSAG
EEKEKERLKATLTKKESFLHITAPKPEDSATYLCAALDRDNYGQNFVFGPGTRL
SVLPY
(SOX2 TCR#11 CDR1α, amino acid)
SEQ ID NO.: 115
VSGLRG
(SOX2 TCR#11 CDR2α, amino acid)
SEQ ID NO.: 116
LYSAGEE
(SOX2 TCR#11 CDR3α, amino acid)
SEQ ID NO.: 117
AALDRDNYGQNFV
(SOX2 TCR#11 CDR3α-junction, amino acid)
SEQ ID NO.: 118
CAALDRDNYGQNFVF
(SOX2 TCR#13 TCRβ-P2A-TCRα, codon-optimized nucleotide)
SEQ ID NO.: 119
ATGACCATTCGACTGCTGTGCTACATGGGCTTCTACTTCCTGGGAGCTGGACTGA
TGGAAGCCGACATCTACCAAACACCCAGATACCTGGTGATCGGAACAGGCA
AGAAGATCACCCTGGAGTGTAGCCAGACAATGGGACACGACAAGATGTACT
GGTACCAGCAGGATCCTGGAATGGAACTGCACCTGATCCACTACAGCTATG
GCGTGAACAGCACAGAGAAAGGCGATCTGAGCAGCGAGTCTACAGTGAGC
AGAATCAGAACCGAGCACTTCCCTCTGACACTGGAATCTGCTAGACCTAGC
CACACAAGCCAGTACCTGTGTGCTTCTAGCGAGAACAGCAATCAGCCCCAG
CATTTCGGCGATGGCACCCGCTTGTCCATCTTGGAGGATCTGAACAAGGTGT
TCCCCCCAGAGGTGGCCGTGTTCGAGCCTTCTGAGGCCGAGATCTCCCACAC
CCAGAAAGCCACCCTCGTGTGCCTGGCCACCGGCTTTTTCCCCGACCACGTG
GAACTGTCTTGGTGGGTCAACGGCAAAGAGGTGCACTCCGGCGTGTGCACC
GATCCCCAGCCTCTGAAAGAACAGCCCGCCCTGAACGACAGCCGGTACTGC
CTGAGCAGCAGACTGAGAGTGTCCGCCACCTTCTGGCAGAACCCCCGGAAC
CACTTCAGATGCCAGGTGCAGTTCTACGGCCTGAGCGAGAACGACGAGTGG
ACCCAGGACAGAGCCAAGCCCGTGACACAGATCGTGTCTGCCGAAGCCTGG
GGCAGAGCCGATTGCGGCTTTACCTCCGTGTCCTATCAGCAGGGCGTGCTG
AGCGCCACAATCCTGTACGAGATCCTGCTGGGCAAGGCCACCCTGTACGCC
GTGCTGGTGTCTGCCCTGGTGCTGATGGCCATGGTCAAGCGGAAGGACTTC
GGTTCCGGAGCCACGAACTTCTCTCTGTTAAAGCAAGCAGGAGACGTGGAA
GAAAACCCCGGTCCCATGACCAGCATTAGAGCCGTGTTCATCTTCCTGTGGCTG
CAGCTGGATCTGGTGAATGGCGAGAATGTGGAACAGCATCCTAGCACACTGT
CTGTTCAGGAGGGCGATTCTGCCGTGATCAAGTGCACCTATAGCGATTCTGC
CAGCAACTACTTCCCTTGGTACAAGCAGGAGCTGGGCAAAGGACCTCAGCT
GATCATCGACATCAGGAGCAATGTGGGAGAGAAGAAGGATCAGAGAATCG
CCGTGACATTAAATAAGACCGCCAAGCACTTTAGCCTGCACATCACAGAGA
CACAACCTGAGGATTCTGCCGTGTACTTTTGTGCCGCCCCTAACGACATGAG
ATTCGGCGCCGGGACACGTCTGACAGTGAAGCCCAACATCCAAAATCCTGA
TCCCGCCGTGTACCAGCTGCGGGACAGCAAGAGCAGCGACAAGAGCGTGTG
CCTGTTCACCGACTTCGACAGCCAGACCAACGTGTCCCAGAGCAAGGACAG
CGACGTGTACATCACCGATAAGTGCGTGCTGGACATGCGGAGCATGGACTT
CAAGAGCAACAGCGCCGTGGCCTGGTCCAACAAGAGCGACTTCGCCTGCGC
CAACGCCTTCAACAACAGCATTATCCCCGAGGACACATTCTTCCCAAGCCCC
GAGAGCAGCTGCGACGTGAAGCTGGTGGAAAAGAGCTTCGAGACAGACAC
CAACCTGAACTTCCAGAACCTCAGCGTGATCGGCTTCCGGATCCTGCTGCTG
AAGGTGGCCGGCTTCAACCTGCTGATGACCCTGCGGCTGTGGTCCAGCTGA
(SOX2 TCR#13 TCRβ-P2A-TCRα, amino acid)
SEQ ID NO.: 120
MTIRLLCYMGFYFLGAGLMEADIYQTPRYLVIGTGKKITLECSQTMGHDKMYWY
QQDPGMELHLIHYSYGVNSTEKGDLSSESTVSRIRTEHFPLTLESARPSHTSQYL
CASSENSNQPQHFGDGTRLSILEDLNKVFPPEVAVFEPSEAEISHTQKATLVCLA
TGFFPDHVELSWWVNGKEVHSGVCTDPQPLKEQPALNDSRYCLSSRLRVSATF
WQNPRNHFRCQVQFYGLSENDEWTQDRAKPVTQIVSAEAWGRADCGFTSVSY
QQGVLSATILYEILLGKATLYAVLVSALVLMAMVKRKDFGSGATNFSLLKQAG
DVEENPGPMTSIRAVFIFLWLQLDLVNGENVEQHPSTLSVQEGDSAVIKCTYSDS
ASNYFPWYKQELGKGPQLIIDIRSNVGEKKDQRIAVTLNKTAKHFSLHITETQPE
DSAVYFCAAPNDMRFGAGTRLTVKPNIQNPDPAVYQLRDSKSSDKSVCLFTDF
DSQTNVSQSKDSDVYITDKCVLDMRSMDFKSNSAVAWSNKSDFACANAFNNS
IIPEDTFFPSPESSCDVKLVEKSFETDTNLNFQNLSVIGFRILLLKVAGFNLLMTL
RLWSS
(SOX2 TCR#13 TCRVβ, amino acid)
SEQ ID NO.: 121
EADIYQTPRYLVIGTGKKITLECSQTMGHDKMYWYQQDPGMELHLIHYSYGV
NSTEKGDLSSESTVSRIRTEHFPLTLESARPSHTSQYLCASSENSNQPQHFGDGT
RLSILE
(SOX2 TCR#13 CDR1β, amino acid)
SEQ ID NO.: 122
MGHDK
(SOX2 TCR#13 CDR2β, amino acid)
SEQ ID NO.: 123
SYGVNS
(SOX2 TCR#13 CDR3β, amino acid)
SEQ ID NO.: 124
ASSENSNQPQH
(SOX2 TCR#13 CDR3β-junction, amino acid)
SEQ ID NO.: 125
CASSENSNQPQHF
(SOX2 TCR#13 TCRVα, amino acid)
SEQ ID NO.: 126
GENVEQHPSTLSVQEGDSAVIKCTYSDSASNYFPWYKQELGKGPQLIIDIRSNV
GEKKDQRIAVTLNKTAKHFSLHITETQPEDSAVYFCAAPNDMRFGAGTRLTVK
PN
(SOX2 TCR#13 CDR1α, amino acid)
SEQ ID NO.: 127
DSASNY
(SOX2 TCR#13 CDR2α, amino acid)
SEQ ID NO.: 128
IRSNVGE
(SOX2 TCR#13 CDR3α, amino acid)
SEQ ID NO.: 129
AAPNDMR
(SOX2 TCR#13 CDR3α-junction, amino acid)
SEQ ID NO.: 130
CAAPNDMRF
(SOX2 TCR#15 TCRβ-P2A-TCRα, codon-optimized nucleotide)
SEQ ID NO.: 131
ATGGGCACATCTCTTCTCTGTTGGGTTGTGCTGGGCTTTCTGGGCACAGATCATA
CAGGAGCTGGAGTTAGCCAGTCTCCTAGGTATAAGGTGACCAAGAGGGGAC
AGGATGTGGCTCTGAGATGTGACCCTATTTCTGGCCACGTGAGCCTTTACTG
GTACAGACAAGCTCTGGGACAAGGACCCGAGTTTCTGACCTACTTCAACTA
CGAGGCCCAGCAGGATAAATCTGGACTGCCCAACGACAGATTCAGCGCCGA
AAGACCTGAAGGCTCTATCAGCACACTGACCATTCAGAGAACAGAGCAGAG
GGATTCTGCCATGTACAGATGCGCCAGCTCTCTTTGGGGAAGAGCCGTTGAT
GAGCAGTTTTTTGGCCCTGGAACCAGACTGACAGTTCTGGAGGACCTGAAG
AACGTGTTCCCCCCAGAGGTGGCCGTGTTCGAGCCTAGCGAGGCCGAGATC
AGCCACACCCAGAAAGCCACCCTCGTGTGCCTGGCCACCGGCTTTTACCCC
GACCACGTGGAACTGTCTTGGTGGGTCAACGGCAAAGAGGTGCACAGCGGC
GTCTGCACCGACCCCCAGCCCCTGAAAGAGCAGCCCGCCCTGAACGACAGC
CGGTACTGTCTGAGCAGCAGACTGAGAGTGTCCGCCACCTTCTGGCAGAAC
CCCCGGAACCACTTCAGATGCCAGGTGCAGTTCTACGGCCTGAGCGAGAAC
GACGAGTGGACCCAGGACCGGGCCAAGCCCGTGACCCAGATCGTGTCTGCT
GAGGCCTGGGGCAGAGCCGATTGCGGCTTCACCAGCGAGAGCTACCAGCAG
GGCGTGCTGAGCGCCACCATCCTGTACGAGATCCTGCTGGGCAAGGCCACC
CTGTACGCCGTGCTGGTGTCCGCCCTGGTGCTGATGGCCATGGTCAAGCGG
AAGGACAGCCGGGGCGGTTCCGGAGCCACGAACTTCTCTCTGTTAAAGCAA
GCAGGAGACGTGGAAGAAAACCCCGGTCCCATGAACTACTCTCCTGGCCTTG
TCAGCCTGATTCTGCTGCTGCTGGGAAGAACAAGAGGCGATAGCGTTACACAG
ATGGAAGGACCTGTGACACTGTCTGAAGAGGCCTTCCTGACCATCAATTGC
ACCTACACAGCCACTGGTTACCCGTCCCTTTTCTGGTATGTGCAGTACCCTG
GAGAGGGACTGCAACTGCTGTTAAAGGCCACAAAAGCCGACGATAAGGGC
AGCAATAAGGGCTTTGAGGCCACCTACAGGAAGGAGACAACCAGCTTTCAT
CTGGAGAAGGGAAGCGTGCAAGTGTCTGATTCAGCCGTGTACTTTTGTGCC
CTGCTGGGCTCTAATGACTACAAGCTGAGCTTTGGAGCCGGCACAACAGTG
ACCGTTAGAGCCAATATCCAAAACCCCGACCCAGCTGTGTACCAGCTGCGG
GACAGCAAGAGCAGCGACAAGAGCGTGTGCCTGTTCACCGACTTCGACAGC
CAGACCAACGTGTCCCAGAGCAAGGACAGCGACGTGTACATCACCGATAAG
TGCGTGCTGGACATGCGGAGCATGGACTTCAAGAGCAACAGCGCCGTGGCC
TGGTCCAACAAGAGCGACTTCGCCTGCGCCAACGCCTTCAACAACAGCATT
ATCCCCGAGGACACATTCTTCCCAAGCCCCGAGAGCAGCTGCGACGTGAAG
CTGGTGGAAAAGAGCTTCGAGACAGACACCAACCTGAACTTCCAGAACCTC
AGCGTGATCGGCTTCCGGATCCTGCTGCTGAAGGTGGCCGGCTTCAACCTGC
TGATGACCCTGCGGCTGTGGTCCAGCTGA
(SOX2 TCR#15 TCRβ-P2A-TCRα, amino acid)
SEQ ID NO.: 132
MGTSLLCWVVLGFLGTDHTGAGVSQSPRYKVTKRGQDVALRCDPISGHVSLYW
YRQALGQGPEFLTYFNYEAQQDKSGLPNDRFSAERPEGSISTLTIQRTEQRDSA
MYRCASSLWGRAVDEQFFGPGTRLTVLEDLKNVFPPEVAVFEPSEAEISHTQKA
TLVCLATGFYPDHVELSWWVNGKEVHSGVCTDPQPLKEQPALNDSRYCLSSRL
RVSATFWQNPRNHFRCQVQFYGLSENDEWTQDRAKPVTQIVSAEAWGRADCG
FTSESYQQGVLSATILYEILLGKATLYAVLVSALVLMAMVKRKDSRGGSGATN
FSLLKQAGDVEENPGPMNYSPGLVSLILLLLGRTRGDSVTQMEGPVTLSEEAFLTI
NCTYTATGYPSLFWYVQYPGEGLQLLLKATKADDKGSNKGFEATYRKETTSF
HLEKGSVQVSDSAVYFCALLGSNDYKLSFGAGTTVTVRANIQNPDPAVYQLRD
SKSSDKSVCLFTDFDSQTNVSQSKDSDVYITDKCVLDMRSMDFKSNSAVAWSN
KSDFACANAFNNSIIPEDTFFPSPESSCDVKLVEKSFETDTNLNFQNLSVIGFRILL
LKVAGFNLLMTLRLWSS
(SOX2 TCR#15 TCRVβ, amino acid)
SEQ ID NO.: 133
GAGVSQSPRYKVTKRGQDVALRCDPISGHVSLYWYRQALGQGPEFLTYFNYE
AQQDKSGLPNDRFSAERPEGSISTLTIQRTEQRDSAMYRCASSLWGRAVDEQFF
GPGTRLTVLE
(SOX2 TCR#15 CDR1β, amino acid)
SEQ ID NO.: 134
SGHVS
(SOX2 TCR#15 CDR2β, amino acid)
SEQ ID NO.: 135
FNYEAQ
(SOX2 TCR#15 CDR3β, amino acid)
SEQ ID NO.: 136
ASSLWGRAVDEQF
(SOX2 TCR#15 CR3β-junction, amino acid)
SEQ ID NO.: 137
CASSLWGRAVDEQFF
(SOX2 TCR#15 TCRVα, amino acid)
SEQ ID NO.: 138
GDSVTQMEGPVTLSEEAFLTINCTYTATGYPSLFWYVQYPGEGLQLLLKATKA
DDKGSNKGFEATYRKETTSFHLEKGSVQVSDSAVYFCALLGSNDYKLSFGAGT
TVTVRAN
(SOX2 TCR#15 CDR1α, amino acid)
SEQ ID NO.: 139
ATGYPS
(SOX2 TCR#15 CDR2α, amino acid)
SEQ ID NO.: 140
ATKADDK
(SOX2 TCR#15 CDR3α, amino acid)
SEQ ID NO.: 141
ALLGSNDYKLS
(SOX2 TCR#15 CDR3α-junction, amino acid)
SEQ ID NO.: 142
CALLGSNDYKLSF
(SOX2 TCR#16 TCRβ-P2A-TCRα, codon-optimized nucleotide)
SEQ ID NO.: 143
ATGTCTATCGGTCTGCTGTGCTGTGTAGCATTCAGTCTACTTTGGGCTTCTCCCG
TGAATGCTGGAGTTACACAAACCCCCAAGTTCCAGGTGTTAAAGACCGGCC
AGAGCATGACACTGCAGTGTGCTCAGGACATGAATCACAACAGTATGTATT
GGTACAGACAGGATCCCGGCATGGGACTGAGACTGATCTACTACAGCGCTT
CTGAGGGAACTACAGACAAAGGAGAGGTGCCCAACGGCTACAACGTGAGC
AGGCTGAACAAGAGGGAGTTTAGCCTGAGACTGGAATCCGCTGCCCCGTCT
CAGACAAGCGTGTACTTCTGCGCCTCTTCTGAGGGATACGGCTATGAGCAG
TATTTTGGCCCTGGCACAAGACTGACAGTGACAGAGGACCTGAAGAACGTG
TTCCCCCCAGAGGTGGCCGTGTTCGAGCCTAGCGAGGCCGAGATCAGCCAC
ACCCAGAAAGCCACCCTCGTGTGCCTGGCCACCGGCTTTTACCCCGACCAC
GTGGAACTGTCTTGGTGGGTCAACGGCAAAGAGGTGCACAGCGGCGTCTGC
ACCGACCCCCAGCCCCTGAAAGAGCAGCCCGCCCTGAACGACAGCCGGTAC
TGTCTGAGCAGCAGACTGAGAGTGTCCGCCACCTTCTGGCAGAACCCCCGG
AACCACTTCAGATGCCAGGTGCAGTTCTACGGCCTGAGCGAGAACGACGAG
TGGACCCAGGACCGGGCCAAGCCCGTGACCCAGATCGTGTCTGCTGAGGCC
TGGGGCAGAGCCGATTGCGGCTTCACCAGCGAGAGCTACCAGCAGGGCGTG
CTGAGCGCCACCATCCTGTACGAGATCCTGCTGGGCAAGGCCACCCTGTAC
GCCGTGCTGGTGTCCGCCCTGGTGCTGATGGCCATGGTCAAGCGGAAGGAC
AGCCGGGGCGGTTCCGGAGCCACGAACTTCTCTCTGTTAAAGCAAGCAGGA
GACGTGGAAGAAAACCCCGGTCCCATGCTGCTGGAACACCTACTGATCATCCT
GTGGATGCAGCTGACATGGGTGTCTGGACAGCAGCTGAATCAGTCTCCTCAG
AGCATGTTCATTCAGGAGGGAGAGGATGTGAGCATGAACTGCACATCGTCC
AGCATCTTCAACACCTGGCTGTGGTATAAGCAGGATCCTGGAGAAGGACCT
GTGCTGCTGATTGCCCTGTATAAAGCCGGAGAGCTGACCTCTAATGGAAGA
CTGACAGCCCAGTTTGGCATCACCAGAAAGGACAGCTTCCTGAACATCTCC
GCATCCATTCCTTCTGATGTGGGCATCTACTTCTGTGCCGGCCACATTACAC
AAGGCGGATCTGAGAAGCTGGTGTTTGGCAAAGGCACCAAGCTGACAGTTA
ACCCCTACATCCAGAATCCCGATCCTGCCGTGTACCAGCTGCGGGACAGCA
AGAGCAGCGACAAGAGCGTGTGCCTGTTCACCGACTTCGACAGCCAGACCA
ACGTGTCCCAGAGCAAGGACAGCGACGTGTACATCACCGATAAGTGCGTGC
TGGACATGCGGAGCATGGACTTCAAGAGCAACAGCGCCGTGGCCTGGTCCA
ACAAGAGCGACTTCGCCTGCGCCAACGCCTTCAACAACAGCATTATCCCCG
AGGACACATTCTTCCCAAGCCCCGAGAGCAGCTGCGACGTGAAGCTGGTGG
AAAAGAGCTTCGAGACAGACACCAACCTGAACTTCCAGAACCTCAGCGTGA
TCGGCTTCCGGATCCTGCTGCTGAAGGTGGCCGGCTTCAACCTGCTGATGAC
CCTGCGGCTGTGGTCCAGCTGA
(SOX2 TCR#16 TCRβ-P2A-TCRα, amino acid)
SEQ ID NO.: 144
MSIGLLCCVAFSLLWASPVNAGVTQTPKFQVLKTGQSMTLQCAQDMNHNSMYW
YRQDPGMGLRLIYYSASEGTTDKGEVPNGYNVSRLNKREFSLRLESAAPSQTSV
YFCASSEGYGYEQYFGPGTRLTVTEDLKNVFPPEVAVFEPSEAEISHTQKATLV
CLATGFYPDHVELSWWVNGKEVHSGVCTDPQPLKEQPALNDSRYCLSSRLRVS
ATFWQNPRNHFRCQVQFYGLSENDEWTQDRAKPVTQIVSAEAWGRADCGFTS
ESYQQGVLSATILYEILLGKATLYAVLVSALVLMAMVKRKDSRGGSGATNFSL
LKQAGDVEENPGPMLLEHLLIILWMQLTWVSGQQLNQSPQSMFIQEGEDVSMNC
TSSSIFNTWLWYKQDPGEGPVLLIALYKAGELTSNGRLTAQFGITRKDSFLNISA
SIPSDVGIYFCAGHITQGGSEKLVFGKGTKLTVNPYIQNPDPAVYQLRDSKSSDK
SVCLFTDFDSQTNVSQSKDSDVYITDKCVLDMRSMDFKSNSAVAWSNKSDFAC
ANAFNNSIIPEDTFFPSPESSCDVKLVEKSFETDTNLNFQNLSVIGFRILLLKVAG
FNLLMTLRLWSS
(SOX2 TCR#16 TCRVβ, amino acid)
SEQ ID NO.: 145
NAGVTQTPKFQVLKTGQSMTLQCAQDMNHNSMYWYRQDPGMGLRLIYYSAS
EGTTDKGEVPNGYNVSRLNKREFSLRLESAAPSQTSVYFCASSEGYGYEQYFGP
GTRLTVTE
(SOX2 TCR#16 CDR1β, amino acid)
SEQ ID NO.: 146
MNHNS
(SOX2 TCR#16 CDR2β, amino acid)
SEQ ID NO.: 147
SASEGT
(SOX2 TCR#16 CDR3β, amino acid)
SEQ ID NO.: 148
ASSEGYGYEQY
(SOX2 TCR#16 CDR3β-junction, amino acid)
SEQ ID NO.: 149
CASSEGYGYEQYF
(SOX2 TCR#16 TCRVα, amino acid)
SEQ ID NO.: 150
GQQLNQSPQSMFIQEGEDVSMNCTSSSIFNTWLWYKQDPGEGPVLLIALYKAG
ELTSNGRLTAQFGITRKDSFLNISASIPSDVGIYFCAGHITQGGSEKLVFGKGTKL
TVNPY
(SOX2 TCR#16 CDR1α, amino acid)
SEQ ID NO.: 151
SIFNT
(SOX2 TCR#16 CDR2α, amino acid)
SEQ ID NO.: 152
LYKAGEL
(SOX2 TCR#16 CDR3α, amino acid)
SEQ ID NO.: 153
AGHITQGGSEKLV
(SOX2 TCR#16 CDR3α-junction, amino acid)
SEQ ID NO.: 154
CAGHITQGGSEKLVF
(TCR Cα1)
SEQ ID NO.: 155
IQNPDPAVYQ LRDSKSSDKS VCLFTDFDSQ TNVSQSKDSD VYITDKTVLD
MRSMDFKSNS AVAWSNKSDF ACANAFNNSI IPEDTFFPSP ESSCDVKLVE
KSFETDTNLN FQNLSVIGFR ILLLKVAGFN LLMTLRLWSS
(TCR Cβ2)
SEQ ID NO: 156
DLKNVFPPKV AVFEPSEAEI SHTQKATLVC LATGFYPDHV ELSWWVNGKE
VHSGVSTDPQ PLKEQPALND SRYCLSSRLR VSATFWQNPR NHFRCQVQFY
GLSENDEWTQ DRAKPVTQIV SAEAWGRADC GFTSESYQQG VLSATILYEI
LLGKATLYAV LVSALVLMAM VKRKDSRG
(TCR Cβ1)
SEQ ID NO: 157
DLNKVFPPEV AVFEPSEAEI SHTQKATLVC LATGFFPDHV ELSWWVNGKE
VHSGVSTDPQ PLKEQPALND SRYCLSSRLR VSATFWQNPR NHFRCQVQFY
GLSENDEWTQ DRAKPVTQIV SAEAWGRADC GFTSVSYQQG VLSATILYEI
LLGKATLYAV LVSALVLMAM VKRKDF
(CD8 co-receptor α chain, without signal sequence)
SEQ ID NO: 158
SQFRVSPLDRTWNLGETVELKCQVLLSNPTSGCSWLFQPRGAAASPTFLLYLSQ
NKPKAAEGLDTQRFSGKRLGDTFVLTLSDFRRENEGYYFCSALSNSIMYFSHFV
PVFLPAKPTTTPAPRPPTPAPTIASQPLSLRPEACRPAAGGAVHTRGLDFACDIYI
WAPLAGTCGVLLLSLVITLYCNHRNRRRVCKCPRPVVKSGDKPSLSARYV
(CD8 co-receptor β chain isoform 1, without signal sequence)
SEQ ID NO: 159
LQQTPAYIKVQTNKMVMLSCEAKISLSNMRIYWLRQRQAPSSDSHHEFLALWD
SAKGTIHGEEVEQEKIAVFRDASRFILNLTSVKPEDSGIYFCMIVGSPELTFGKGT
QLSVVDFLPTTAQPTKKSTLKKRVCRLPRPETQKGPLCSPITLGLLVAGVLVLL
VSLGVAIHLCCRRRRARLRFMKQFYK
(CD8 co-receptor β chain isoform 2, without signal sequence)
SEQ ID NO: 160
LQQTPAYIKVQTNKMVMLSCEAKISLSNMRIYWLRQRQAPSSDSHHEFLALWD
SAKGTIHGEEVEQEKIAVFRDASRFILNLTSVKPEDSGIYFCMIVGSPELTFGKGT
QLSVVDFLPTTAQPTKKSTLKKRVCRLPRPETQKGPLCSPITLGLLVAGVLVLL
VSLGVAIHLCCRRRRARLRFMKQLRLHPLEKCSRMDY
(CD8 co-receptor β chain isoform 3, without signal sequence)
SEQ ID NO: 161
LQQTPAYIKVQTNKMVMLSCEAKISLSNMRIYWLRQRQAPSSDSHHEFLALWD
SAKGTIHGEEVEQEKIAVFRDASRFILNLTSVKPEDSGIYFCMIVGSPELTFGKGT
QLSVVDFLPTTAQPTKKSTLKKRVCRLPRPETQKGPLCSPITLGLLVAGVLVLL
VSLGVAIHLCCRRRRARLRFMKQKFNIVCLKISGFTTCCCFQILQISREYGFGVL
LQKDIGQ
(CD8 co-receptor β chain isoform 4, without signal sequence)
SEQ ID NO: 162
LQQTPAYIKVQTNKMVMLSCEAKISLSNMRIYWLRQRQAPSSDSHHEFLALWD
SAKGTIHGEEVEQEKIAVFRDASRFILNLTSVKPEDSGIYFCMIVGSPELTFGKGT
QLSVVDFLPTTAQPTKKSTLKKRVCRLPRPETQKGPLCSPITLGLLVAGVLVLL
VSLGVAIHLCCRRRRARLRFMKQKFNIVCLKISGFTTCCCFQILQISREYGFGVL
LQKDIGQ
(CD8 co-receptor β chain isoform 5, without signal sequence)
SEQ ID NO: 163
LQQTPAYIKVQTNKMVMLSCEAKISLSNMRIYWLRQRQAPSSDSHHEFLALWD
SAKGTIHGEEVEQEKIAVFRDASRFILNLTSVKPEDSGIYFCMIVGSPELTFGKGT
QLSVVDFLPTTAQPTKKSTLKKRVCRLPRPETQKGPLCSPITLGLLVAGVLVLL
VSLGVAIHLCCRRRRARLRFMKQPQGEGISGTFVPQCLHGYYSNTTTSQKLLNP
WILKT

### EXAMPLES

### Example 1

### Identification of SOX2 Epitopes and Generation of SOX2 Antigen-Reactive T Cell Lines

Multiple myeloma (MM) results from the uncontrolled growth of clonal antibody-producing plasma cells in the bone marrow. It is the second most common hematologic malignancy in the United States, and is nearly always fatal. Adoptive cell therapy (ACT) is an emerging treatment for MM, but work is ongoing to provide a reproducibly effective

standard of care. Chimeric antigen receptor (CAR) T cells targeting cell-surface CD19, BCMA, or CD138 antigens expressed on MM cell surface have had some success. Unfortunately, many patients relapse due to outgrowth of antigen-negative MM cells. Targeting a protein involved in the induction or maintenance of the malignant phenotype may be an important approach for successful ACT. The transcription factor SOX2 may regulate cell proliferation and self-renewal in MM and other cancers, and represents a promising target for ACT.

Methods: BIMAS, SYFPEITHI, NetMHC, IEDB and hmMHC (Alspach et al, Nature 2019) epitope prediction algorithms were used to predict whether any SOX2-derived peptides bind favorably to HLA-A*02:01 allele, which is present in ˜40% of the population in the United States (IEDB population data). These in silico efforts identified 17 peptides predicted to bind favorably to HLA-A*02:01.

To generate CD8⁺ T cell lines specific for SOX2, CD8⁺ T cells from ten healthy HLA-A*02:01⁺ donors were co-cultured with autologous dendritic cells pulsed with SOX2 peptides that are predicted to bind avidly to the HLA-A*02:01 allele. This process yielded 10 different CD8⁺ T cell lines/donor (100 lines total). After 10-14 days in culture (“Stimulation 1”), each CD8⁺ T cell line was restimulated with autologous peripheral blood mononuclear cells that were irradiated and pulsed with the same set of SOX2-derived peptides (“Stimulation 2”). This was repeated 1-2 more times, for a total of 3-4 rounds of stimulation. Following the last stimulation, T cell lines were tested for responses to the SOX2 peptide pool, individual SOX2 peptides, or bound HLA-A*02:01/SOX2 tetramers. Responding cells were sorted and rapidly expanded for 10-14 days. Following the rapid expansion protocol, target killing assays were performed. These assays are further described below.

### Example 2

### T Cells Respond to Specific SOX2 Epitopes

Methods: CD8⁺ T cell lines were individually stimulated with SOX2 peptide pool to determine if any of the T cell lines contained SOX2-reactive CD8⁺ T cells. Briefly, 50,000-100,000 CD8⁺ T cells from each line were incubated with 1 μg/ml SOX2 peptides, media, or Cell Stimulation Cocktail for 4 hours in the presence of Golgi plug and Golgi block. After the end of the incubation period, cells were stained with anti-CD8 antibody, fixed and permeabilized, and stained with anti-IFNγ antibody. Samples were run on a BD Canto 2 instrument, equipped with 3 laser lines. Data were acquired with the Diva Software (BD Biosciences), and analyzed using FlowJo (FlowJo Inc.).

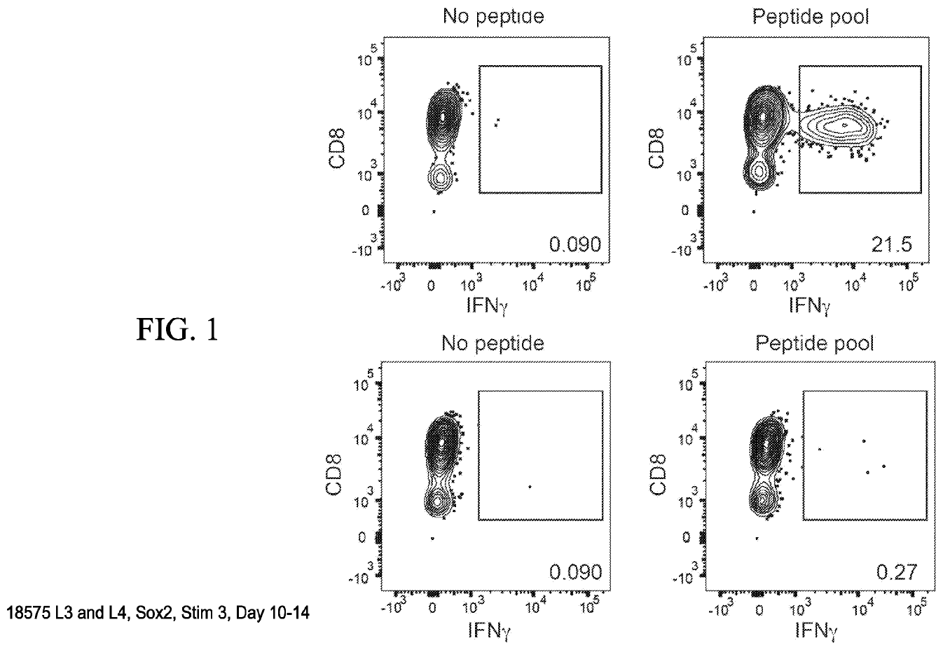

Results: If the percent of IFNγ⁺ CD8⁺ T cells after peptide stimulation was significantly higher than background, that line was considered to be SOX2-reactive. FIG. 1 shows examples of a SOX2-reactive T cell line (21.5% IFNγ⁺ cells; top plots; donor 18575, line 3) and a line that was not considered SOX2-reactive (0.27% IFNγ⁺ cells; bottom plots; donor 18575, line 4). Cell lines that were considered reactive were functionally characterized further. Overall, 49/100 CD8⁺ T cell lines were SOX2-reactive according to this assay.

### Example 3

### Exemplary T Cells Specific for SOX2 Peptides are HLA-A*02:01-Restricted

Confirmed SOX2-specific CD8⁺ T cell lines were examined for HLA-A*02:01-restriction. This was to ensure that the observed results were not due to the T cells using endogenous HLA I scaffold proteins to present SOX2 peptides. To ensure that SOX2 peptide presentation occurred exclusively through HLA-A*02:01, HLA-A*02:01⁺ T2 lymphoblast cells were used as peptide-presenting cells. T2 cells lack a transporter critical for HLA-mediated presentation of endogenous peptides, but can present exogenously provided peptides (provided that the peptides bind HLA avidly; Loft et al, J Immunol 2001).

Methods: 50,000-100,000 CD8⁺ T cells from each line were incubated with 1 μg/ml SOX2 peptides, T2 cells pre-loaded with SOX2 peptides (for at least 1 hour and then washed), pre-loaded T2 cells plus peptides, or media, for 4 hours in the presence of Golgi plug and Golgi block. After the end of the incubation period, the cells were stained with anti-CD8 antibody, fixed and permeabilized, and stained with anti-IFNγ antibody. Samples were run on a BD Canto 2 instrument, equipped with 3 laser lines. Data were acquired with the Diva Software (BD Biosciences), and analyzed using FlowJo (FlowJo Inc.). Shown is the frequency of IFNγ⁺ CD8⁺ T cells after different incubation conditions. Donor and line number is indicated on the slide.

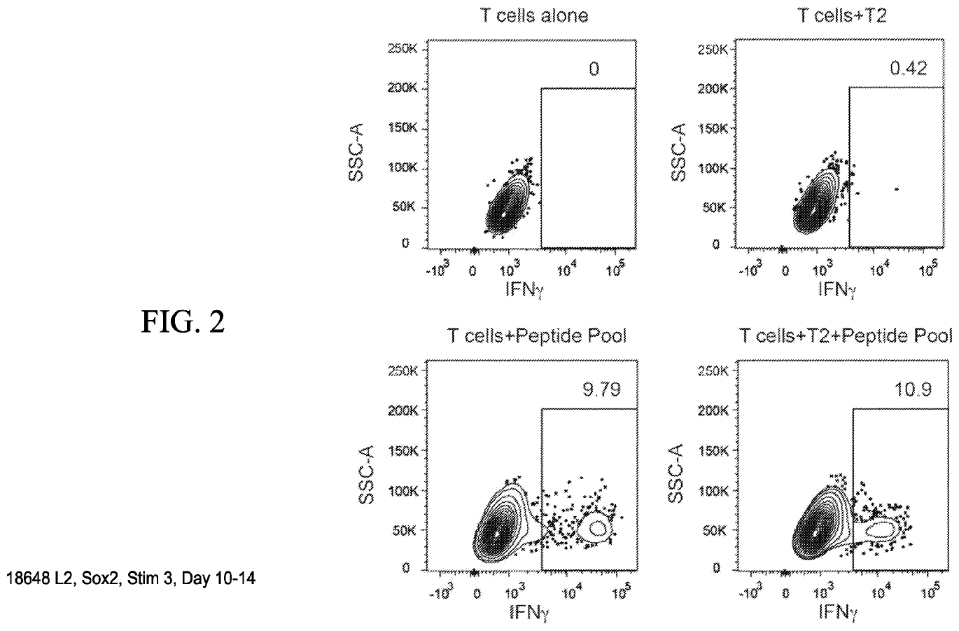

Results: FIG. 2 shows an exemplary CD8⁺ T cell line (donor 18648, line 2) that produced similar amounts of IFNγ when (1) T cells had the opportunity to present SOX2 peptides to each other, and when (2) SOX2 peptide presentation was restricted to T2 cells. This result was interpreted as indicative of T cell line restriction to HLA-A*02:01. Overall, 47/49 SOX2-specific T cell lines were HLA-A*02:01-restricted.

### Example 4

### Validation of SOX2-Specific T Cell Lines

47 CD8⁺ T cell lines that were SOX2-specific and HLA-A*02:01-restricted were studied for recognition of SOX2 peptide(s).

Methods: 50,000-100,000 CD8⁺ T cells were pulsed with individual peptides, and IFNγ production was examined 4 hours later, as described above. Briefly, 50,000-100,000 CD8⁺ T cells from each line were stimulated with 1 μg/ml individual SOX2 peptides, media, or Cell Stimulation Cocktail for 4 hours in the presence of Golgi plug and Golgi block. After the end of the incubation period, the cells were stained with anti-CD8 antibody, fixed and permeabilized, and stained with anti-IFNγ antibody. Samples were run on a BD Canto 2 instrument, equipped with 3 laser lines. Data were acquired with the Diva Software (BD Biosciences), and analyzed using FlowJo (FlowJo Inc.). Shown is the frequency of IFNγ⁺ CD8⁺ T cells. Donor and line numbers, and corresponding stimulation conditions are indicated.

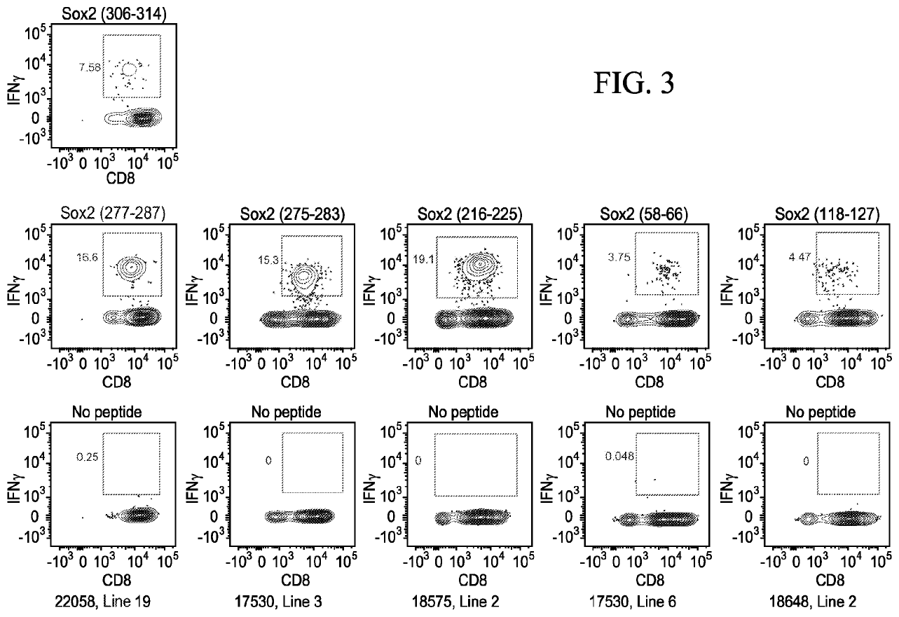

Results: CD8⁺ T cell lines responded to six peptides. FIG. 3 provides example plots of IFNγ⁺ CD8⁺ T cells in the presence of media or indicated SOX2 peptide. From this work, the following six peptides were identified as potentially immunogenic in the context of HLA-A*02:01:

SOX2 peptide (58-66)
(SEQ ID NO.: 6)
KMAQENPKM
SOX2 peptide (118-127)
(SEQ ID NO.: 7)
TLMKKDKYTL
SOX2 peptide (216-225)
(SEQ ID NO.: 3)
YMNGSPTYSM
SOX2 peptide (275-283)
(SEQ ID NO.: 2)
SMYLPGAEV
SOX2 peptide (277-287)
(SEQ ID NO.: 5)
YLPGAEVPEPA
SOX2 peptide (306-314)
(SEQ ID NO.: 4)
TAINGTLPL.

### Example 5

### SOX2 Epitope Processing

The following experiments were performed to determine how SOX2 epitopes are processed and presented.

Multiple myeloma is a plasma cell malignancy, and plasma cells generally express the immunoproteasome (“IP”). However, proteasome inhibitors are sometimes used to treat multiple myeloma patients (Rajkumar et al, Nat Rev Dis Primers 2017), which could alter proteasome subunit expression, influence proteasome catalytic capacity and alter HLA class I ligandome. SOX2 is also expressed in small cell lung cancer cells (Rudin et al, Nat Genet 2012) and glioblastoma cells (Alonso et al, PLOS One 2011). These solid tumor cells likely express the standard proteasome (“SP”), and can activate the immunoproteasome (IP) in the presence of IFNg (produced by T cells).

The SP and the IP cleave peptides differently. Accordingly, proteasome processing could potentially affect breadth, efficacy, and safety of immunotherapy targeting SOX2 peptide:HLA complexes. For example, TCR immunotherapy targeting an SP- and IP-dependent peptide might be beneficial in myeloma and in solid tumors. Such a TCR immunotherapy might still be effective in the case of IP subunit loss (Greenberg and Chapuis et al., unpublished). A TCR immunotherapy that only targets an epitope or epitopes derived from the standard proteasome (SP) processing pathway might be less useful, or not useful, in the context of myeloma, but might be useful in nonhematopoietic malignancies. Conversely, TCR immunotherapy that targets an epitope (or epitopes) derived from the immunoproteasome (IP) processing pathway might be useful in the context of myeloma, but less useful, or not useful, in the context of solid tumors.

To address how SOX2 epitopes were processed, 293E cells modified to express the standard (293E-SP) or the immunoproteasome (293E-IP); (described in Guillame et al, PNAS 2010) were used.

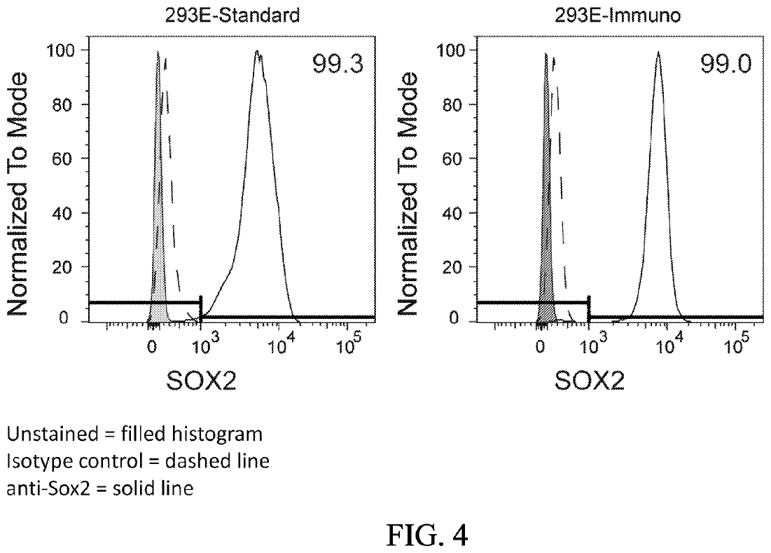

FIG. 4 shows SOX2 expression in 293E-SP and 293-IP cells, as determined by flow cytometry. Briefly, 150,000 cells were fixed and permeabilized, and stained with PE-tagged isotype control or PE-tagged anti-SOX2 antibody. Samples were run on a BD Canto 2 instrument, equipped with 3 laser lines. Data were acquired with the Diva Software (BD Biosciences), and analyzed using FlowJo (FlowJo Inc.). Graphs show the fluorescent staining intensity in the PE channel.

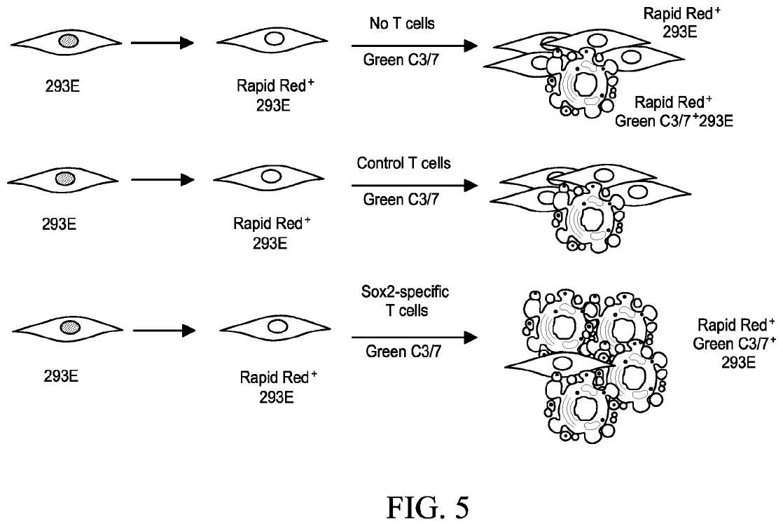

Methods: 293E-SP or 293E-IP cells were used as target cells in an IncuCyte killing assay. FIG. 5 schematically illustrates the experimental design. 293E cells (SP or IP) were labeled with a RapidRed Cytolight Dye (Sartorius) and plated at a density of 5,000 cells/well in a 96-well plate overnight, to promote their adherence to the plate. Added the next day were T cell lines specific for the 6 different SOX2 peptides mentioned in Example 4, or T cells that were not SOX2-specific, but were in the same rapid expansion cycle as the SOX2-specific T cells. A caspase reporter reagent that fluoresces green if caspase 3/7 are active within a cell (Sartorius) was also added. Plates were loaded into an IncuCyte S3 system, and 4 images per well were acquired at 2 hour intervals, over two days. The amount of apoptotic target cells (Cytolight Red⁺ Caspase 3/7 green⁺) was quantified using the IncuCyte S3 software.

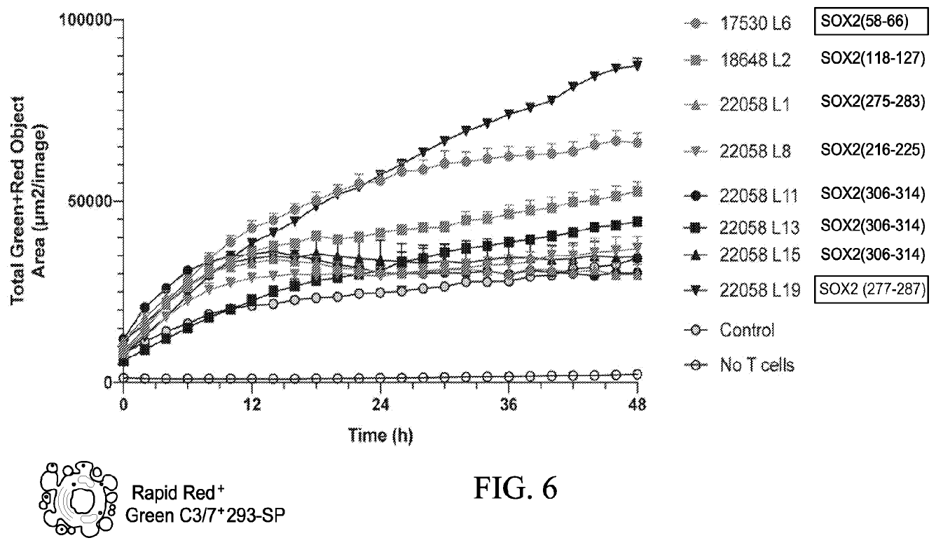

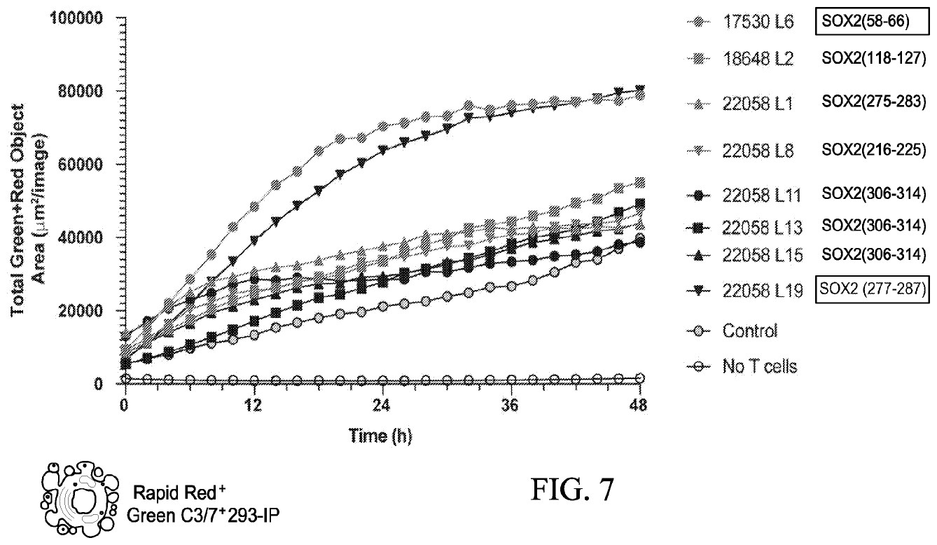

Results: FIG. 6 shows the amount of apoptotic 293E-SP target cells in the presence of SOX2-specific CD8⁺ T cell lines. T cells of irrelevant specificity were included as a control. Boxed text in the figure key indicates two T cell lines that induced the best killing—17530 line 6 (specific for Sox2 58-66) and 22058 line 19 (specific for Sox2 277-287). These results indicate that SOX2 epitopes 58-66 and 277-287 are processed by the standard proteasome (SP). FIG. 7 shows the amount of apoptotic 293E-IP target cells in the presence of SOX2-specific CD8⁺ T cell lines. Boxed text in the figure key indicates two T cell lines that induced the best killing—17530 line 6 (specific for Sox2 58-66) and 22058 line 19 (specific for Sox2 277-287). These results indicate that SOX2 epitopes 58-66 and 277-287 are processed by the immunoproteasome (IP), as well. The SOX2 277-287 peptide sequence is unique to SOX2, and and T cells specific for this epitope were selected for functional characterization.

### Example 6

### SOX2 (277-287)-Specific CD8⁺ T Cells Kill Tumor Cells

To determine if SOX2 (277-287)-specific T cell lines could recognize and kill SOX2-expressing tumor cells, IncuCyte killing assays were performed using T cell lines from HLA-A*02:01 donors expanded with SOX2 (277-287) epitope. Target cells included plasma cell leukemia (late stage myeloma) line L363 and ovarian cancer cell line OVCAR3.

Methods: IncuCyte experiments were performed as described above, and the quantity of apoptotic (Cytolight RapidRed⁺ Caspase 3/7 green⁺) target cells was quantified using the IncuCyte S3 software.

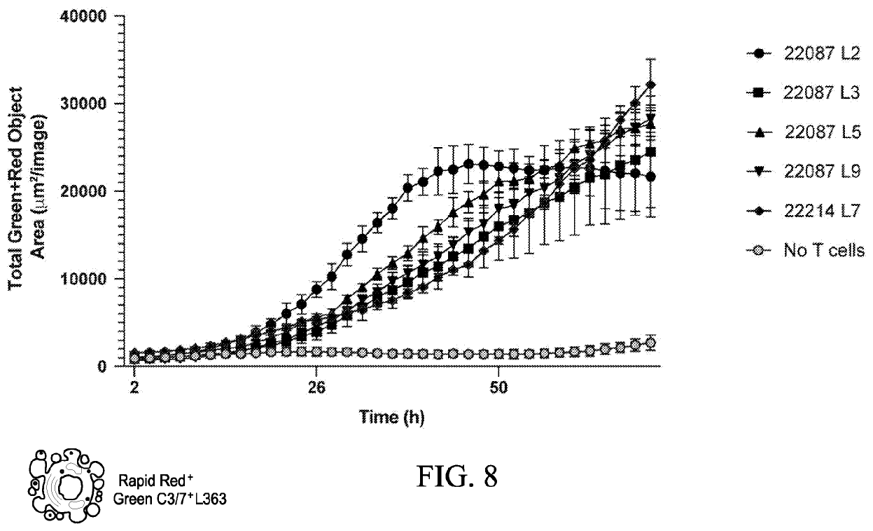

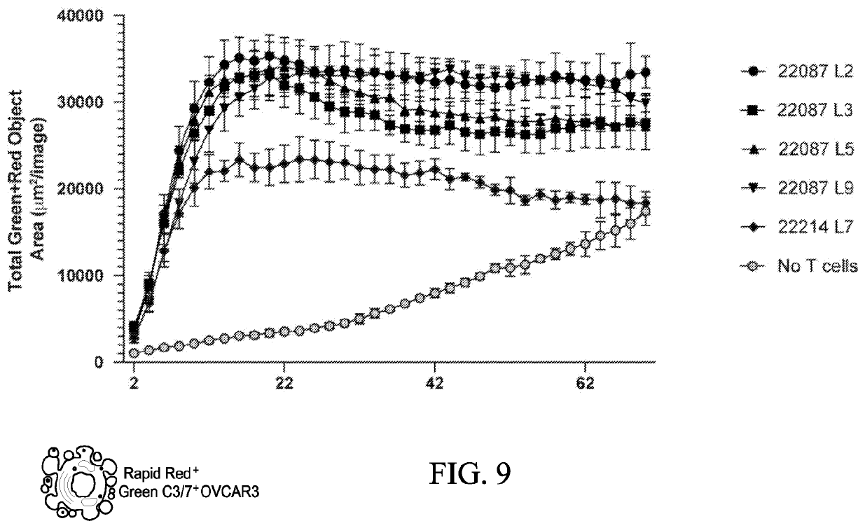

Results: As shown in FIGS. 8 and 9, SOX2 (277-287)-specific T cells induced L363 and OVCAR3 cell apoptosis, suggesting that these T cells specific for this epitope are functionally relevant.

### Example 7

### Sorting and Sequencing of SOX2 (277-287)-Specific T Cells

Prior to sequencing, T cell lines of interest from different donors are pooled, divided into equal parts, and stained with different concentrations of the relevant HLA-A*02:01/peptide tetramer. Tetramer⁺ cells (bulk and top binders in each staining condition) as well as tetramer-negative cells are then sorted and prepared for sequencing (Adaptive Biotechnologies and 10× Genomics). Different tetramer concentrations allow identification of strongly binding TCRs, as those would be enriched in the “top binder” categories, and especially in the staining condition with a lower tetramer concentration.

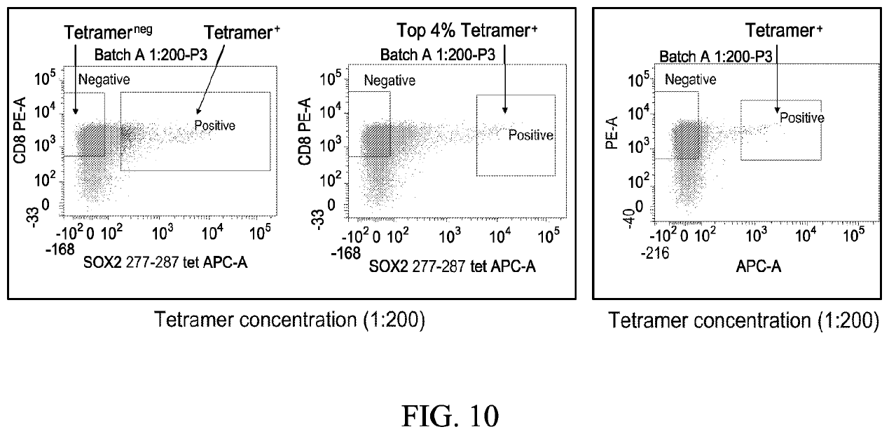

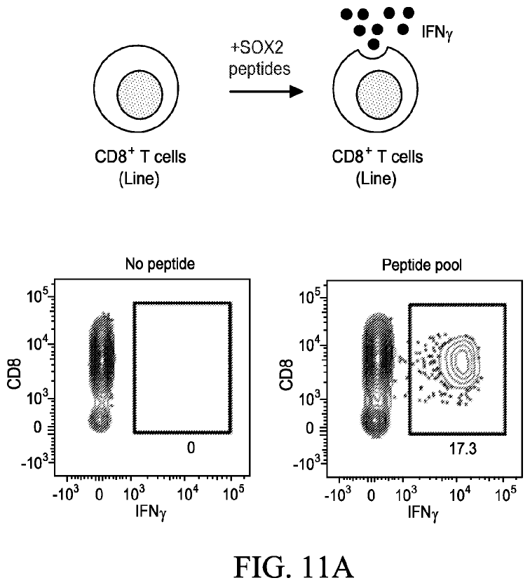

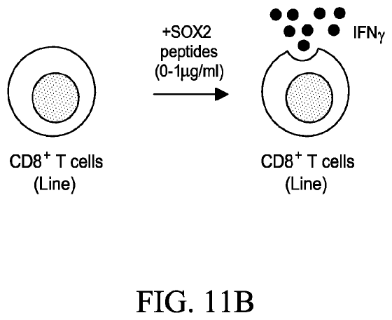

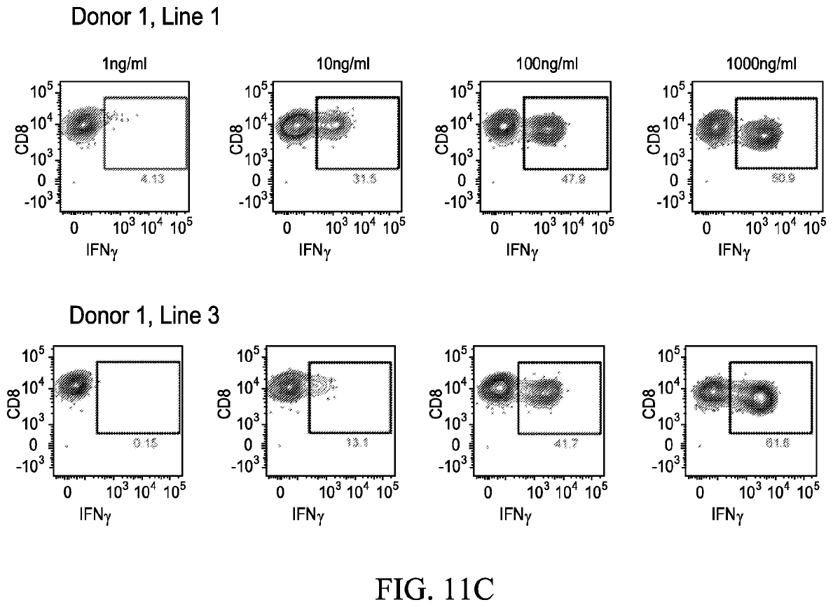

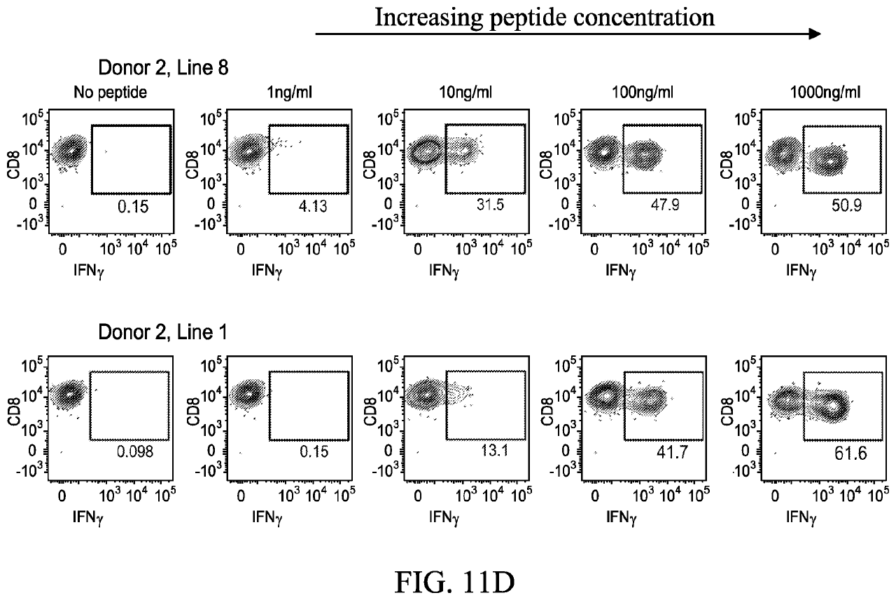

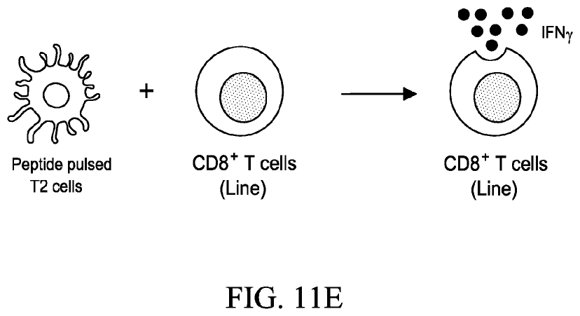

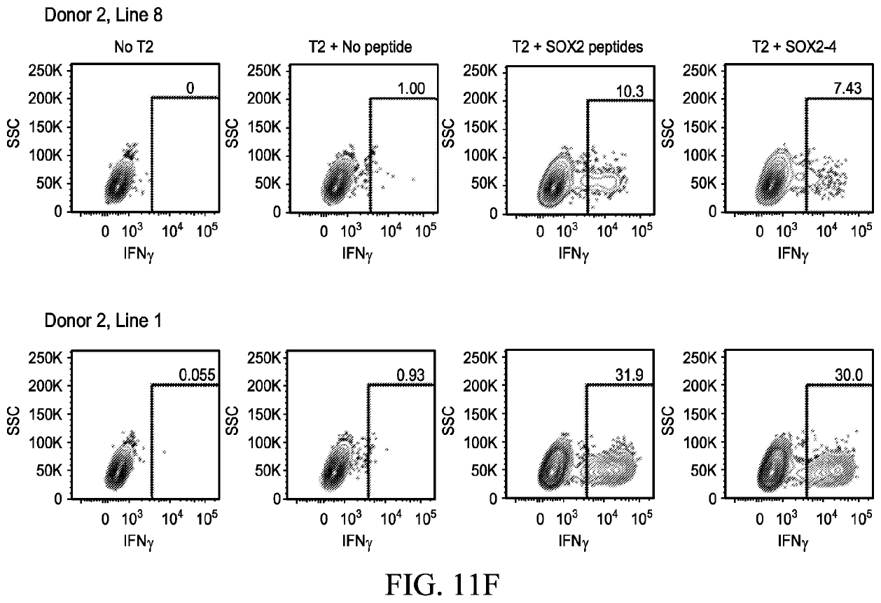

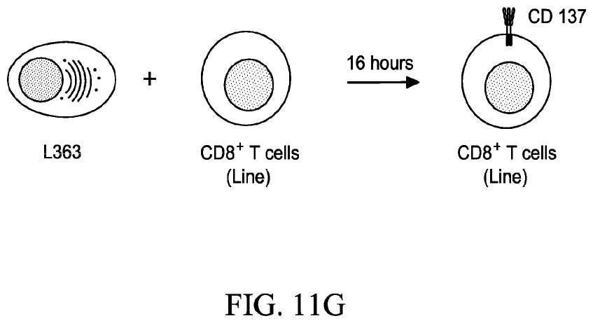

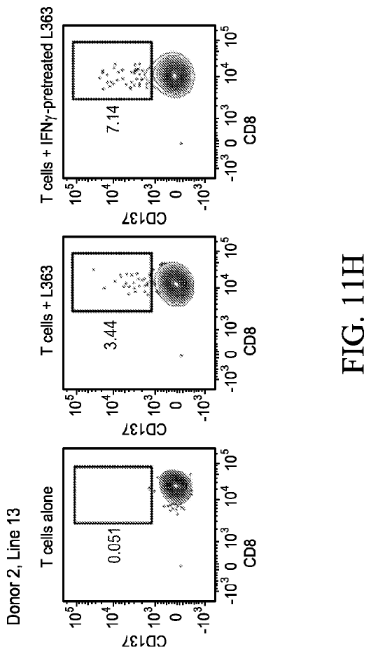

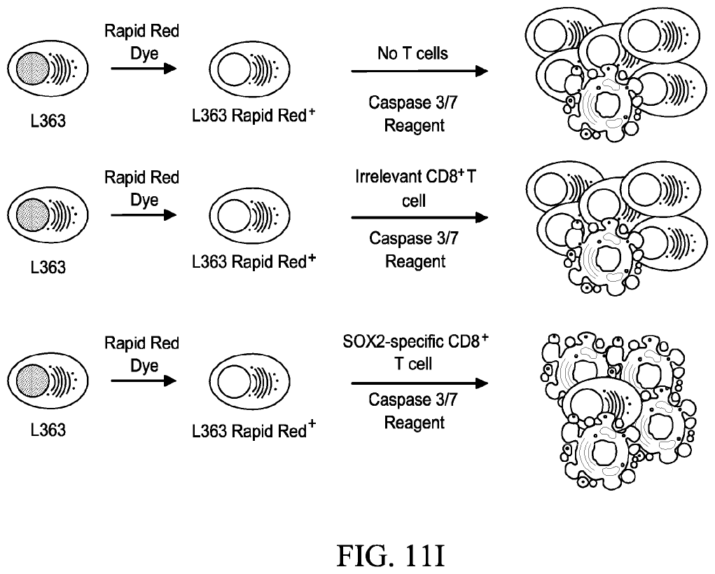

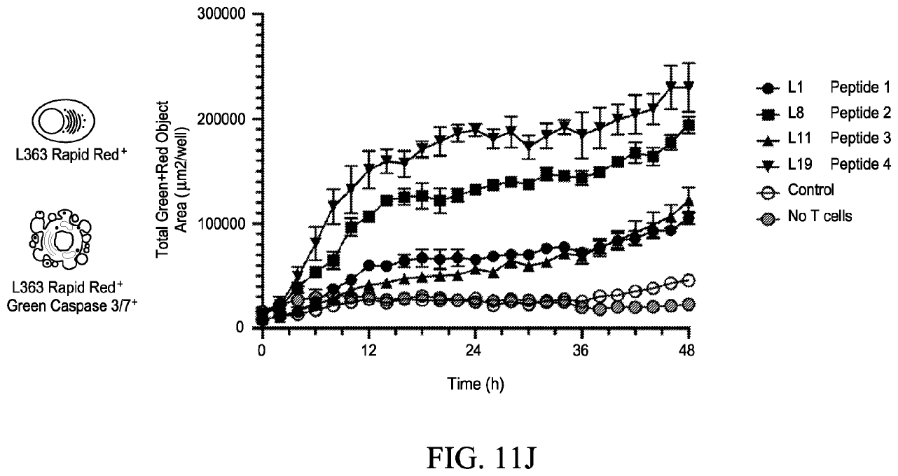

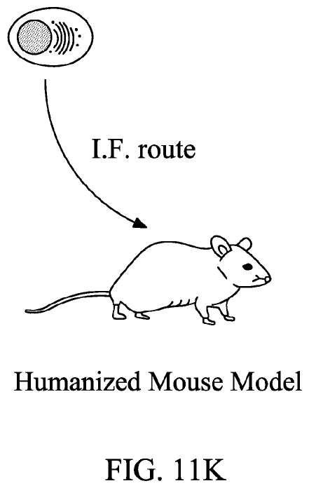

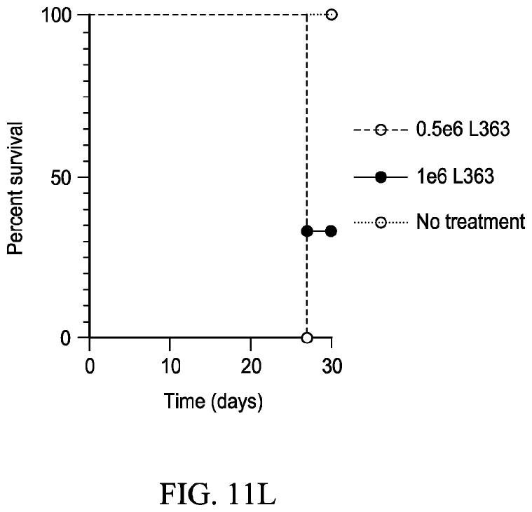

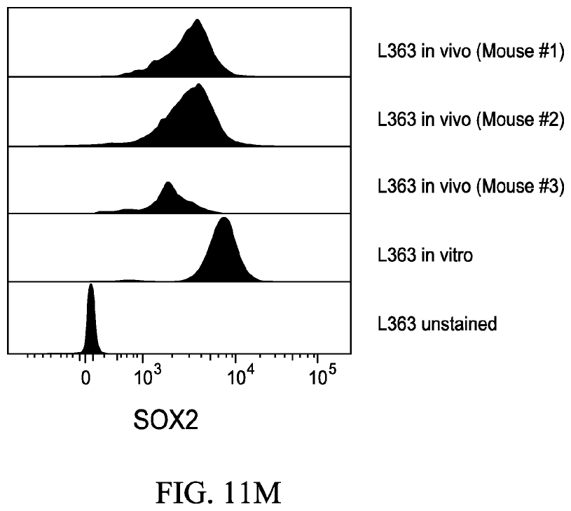

Based on results from HLA-A*02:01/Sox2 (277-287) tetramer titration, a staining and sorting strategy for T cell lines pooled from five donors (17861, 18315, 22058, 22087, 22214) was devised. Cells were stained with 1:200 (optimal tetramer concentration) and 1:2000 (low tetramer concentration) HLA-A*02:01/SOX2 (277-287) tetramer, and sorted. Example data is shown in FIG. 10.

Samples for Adaptive Biotechnologies were frozen after sort (as cell pellets) and samples for 10× Genomics were processed immediately. ImmunoSEQ assay from Adaptive Biotechnologies yielded TCRβ sequences that were found across samples, and allowed calculation of fold enrichment for each TCRβ (identified via unique CDR3 sequence). 10× Genomics sequencing was performed on single cells and allowed pairing each TCRβ that emerged as “highly enriched” in ImmunoSEQ with its corresponding TCRα. TCR gene usage is summarized in Table 1.

TABLE 1
Gene usage of certain SOX2-specific TCRs

| | TRAV/ | | | | | | |
|---|---|---|---|---|---|---|---|
| TCR name | TRDV | TRAJ | TRAC | TRBV | TRBJ | TRBD | TRBC |
| Sox2 TCR | 17*01 | 54*01 | Cα1 | 24-1*01 | 2-3*01 | 2*01 | Cβ2 |
| #01 | | | | | | | |
| Sox2 TCR | 10*01 | 33*01 | Cα1 | 28*01 | 2-2*01 | D2*02 | Cβ2 |
| #02 | | | | | | | |
| Sox2 TCR | 41*01 | 57*01 | Cα1 | 5-5*01 | 2-3*01 | | Cβ2 |
| #04 | | | | | | | |
| Sox2 TCR | 14/4*01 | 8*01 | Cα1 | 28*01 | 1-5*01 | 1*01 | Cβ1 |
| #05 | | | | | | | |
| Sox2 TCR | 6*02 | 17*01 | Cα1 | 20-1*02 | 2-5*01 | 2*01 | Cβ2 |
| #06 | | | | | | | |
| Sox2 TCR | 12-1*01 | 6*01 | Cα1 | 7-2*01 | 2-1*01 | 2*01 | Cβ2 |
| #07 | | | | | | | |
| Sox2 TCR | 12-3*01 | 39*01 | Cα1 | 2*01 | 2-5*01 | 2*01 | Cβ2 |
| #09 | | | | | | | |
| Sox2 TCR | 5*01 | 42*01 | Cα1 | 25-1*01 | 1-1*01 | 1*01 | Cβ1 |
| #10 | | | | | | | |
| Sox2 TCR | 20*02 | 26*01 | Cα1 | 27*01 | 2-1*01 | 2*02 | Cβ2 |
| #11 | | | | | | | |
| Sox2 TCR | 13-1*01 | 43*01 | Cα1 | 25-1*01 | 1-5*01 | | Cβ1 |
| #13 | | | | | | | |
| Sox2 TCR | 9-2*03 | 20*01 | Cα1 | 7-6*01 | 2-1*01 | 2*02 | Cβ2 |
| #15 | | | | | | | |
| Sox2 TCR | 35*02 | 57*01 | Cα1 | 6-1*01 | 2-7*01 | 2*01 | Cβ2 |
| #16 | | | | | | | |

Wild type TCRα and TCRb sequences were codon-optimized, and linked with a P2A self-cleaving peptide. Complementary cysteine residues were incorporated into the TRA and TRB constant domains to increase exogenous TCR pairing and decrease mispairing with endogenous TCR (see, e.g., Dossa et al., Blood 131: 108-120 (2018)).

These TCR expression cassettes were inserted into the pRRLSIN4 vector using Gibson assembly for lentiviral production and testing in primary CD8⁺ T cells.

### Example 8

### Validation of SOX2 (277-287)-Specific TCRs in Primary CD8⁺ T Cells

Methods: TCR expression vectors were sequenced after Gibson assembly to confirm successful insertion of the TCR expression cassettes into the pRRLSIN vector. Lentiviral particles were generated using the 293T/17 packaging cell line and the Effectene kit (Qiagen). CD8⁺ T cells were isolated from a healthy donor (18648) using negative magnetic separation (Easy Sep Kit, STEMCELL Technologies), stimulated for 4 hours with anti-CD3/CD28 beads (Dynabeads), and transduced with SOX2-TCR-encoding lentiviral particles collected from 293T/17 cells. Transduced CD8⁺ T cells were assessed for tetramer staining on day 5 post transduction. SOX2-TCR⁺ CD8⁺ T cells are sorted for rapid expansion, and assessed for response to different peptide concentrations, and target killing.

### Example 9

### L363 Myeloma Cells Exhibit Progressive Tumor Growth in a Mouse Model of Human Immune System Function and Retain SOX2 Expression In Vivo

Humanized mice were injected with 0.5e6 or 1e6 L363 cells via intrafemoral injection and followed for survival. SOX2 expression in bone-marrow infiltrating L363 cells was confirmed by flow cytometry and compared to L363 cells grown in vitro.

### Example 10

### Additional Studies

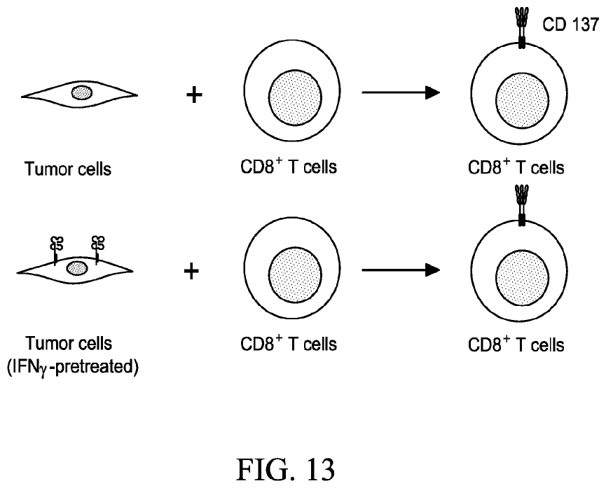

In one experiment, 50,000-100,000 TCR-transduced CD8+ T cells (from two different donors) were stimulated with varying concentrations of SOX2 277-287 peptide (10 μg/ml-0.01 ng/ml), media, or Cell Stimulation Cocktail for 4 hours in the presence of Golgi plug and Golgi block. After the end of the incubation period, the cells were stained with anti-CD8 antibody, fixed and permeabilized, and stained with anti-IFNγ antibody. Samples were run on a BD Canto 2 instrument, equipped with 3 laser lines. Data were acquired with the Diva Software (BD Biosciences), and analyzed using FlowJo (FlowJo Inc.). LogEC50 for each TCR was calculated in Prism (V9, GraphPad) using non-linear regression analysis. Data are shown in FIG. 12. In another experiment, HLA-A2+ tumor cells expressing Sox2 were plated at a 3:1 ratio with Sox2-TCR-transduced CD8+ T cells. In some cases, tumor cells were pre-treated with IFNγ in order to promote increased HLA-A2 expression. Cells were co-cultured overnight (16-18h), and CD137 expression was assessed on CD8+ T cells by flow cytometry. A scheme of the co-culture setup is shown in FIG. 13.

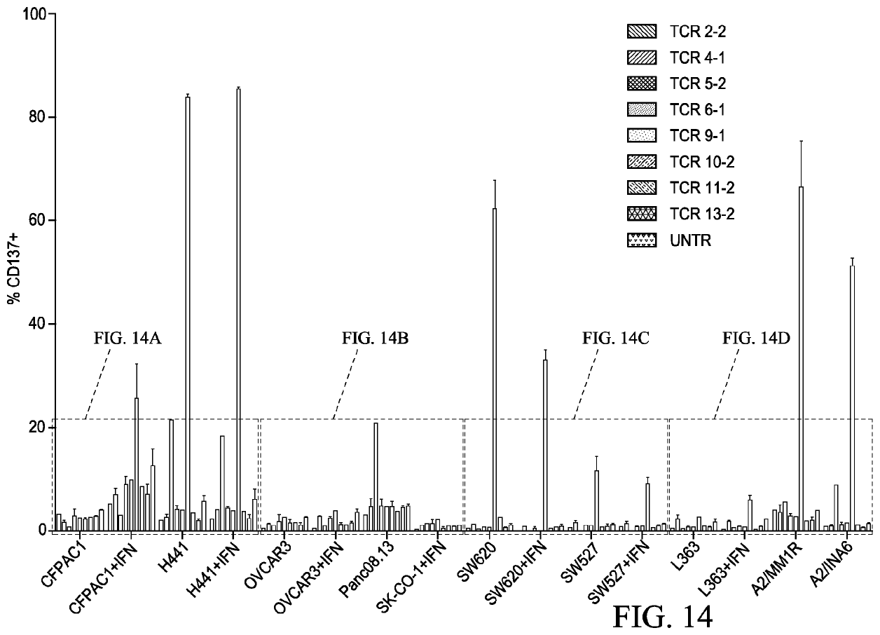

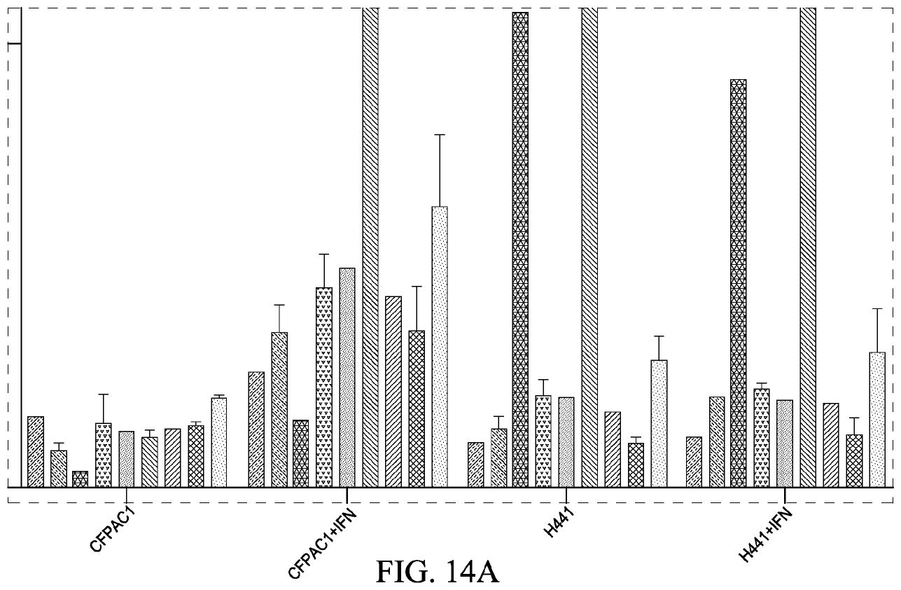

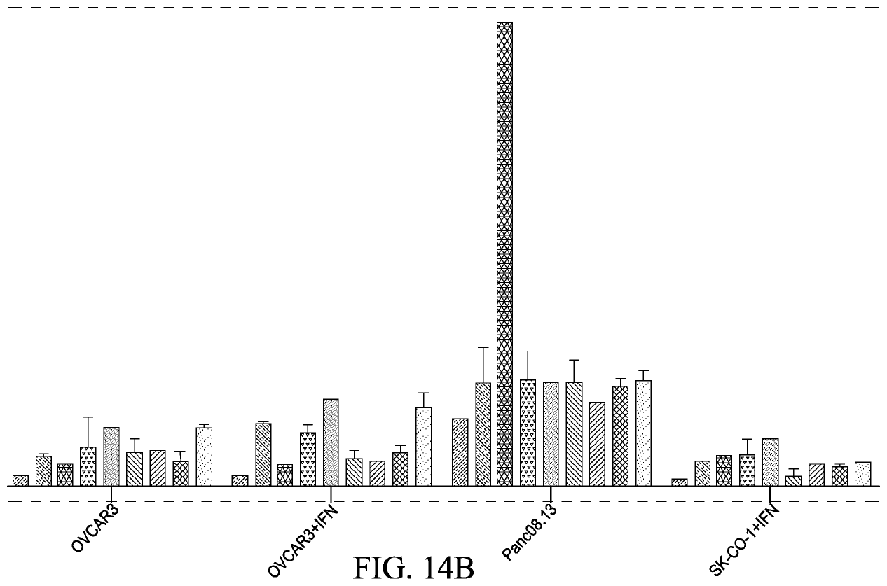

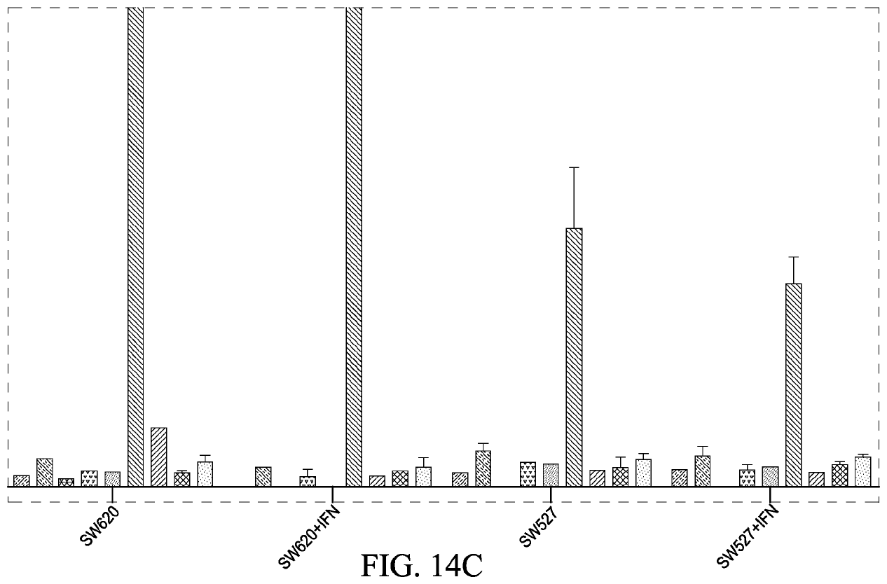

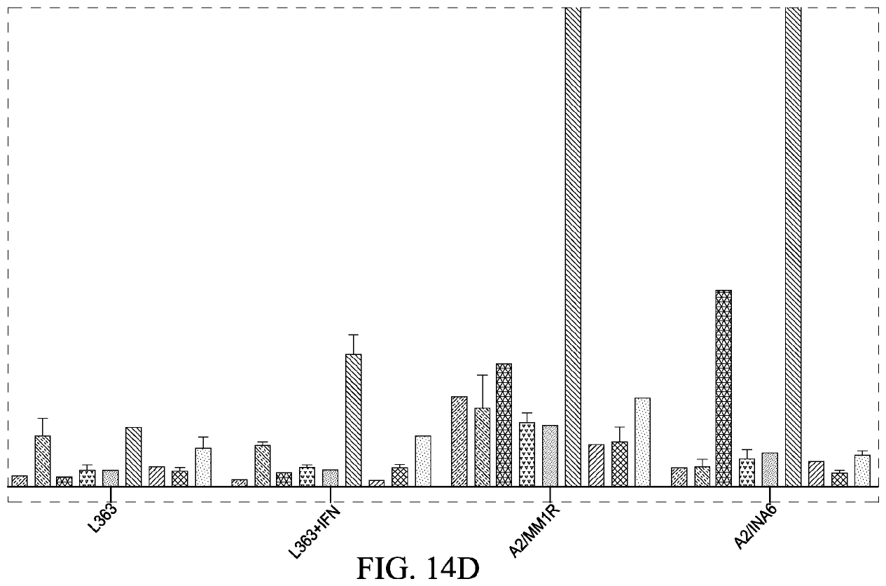

In another experiment, CD8⁺ T cells were transduced with TCR5 or TCR10, and incubated overnight with tumor cells (untreated or pre-treated with IFN-γ) that express HLA-A2 and SOX2. CD137 expression (+/− SEM across two donors) is shown in FIG. 14.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/083,069, filed on Sep. 24, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An isolated binding protein that is capable of binding to a SOX2 peptide antigen:HLA complex, wherein the SOX2 peptide comprises or consists of the amino acid sequence set forth in SEQ ID NO.:5, 2, 3, 4, 6, or 7, and wherein, optionally, the binding comprises specific binding.

2. The binding protein of claim 1, wherein the SOX2 peptide antigen comprises or consists of the amino acid sequence set forth in SEQ ID NO.:5.

3-7. (canceled)

8. The binding protein of claim 1, wherein the HLA comprises HLA-A*02:01.

9. The binding protein of claim 1, comprising an immunoglobulin superfamily variable domain.

10. The binding protein of claim 1, comprising a TCR α-chain variable domain (Vα) and/or a TCR β-chain variable domain (Vβ).

11. The binding protein of claim 1, comprising a heavy chain variable domain (VH) and/or a light chain variable domain (VL) of a TCR-mimic antibody.

12. The binding protein of claim 1, wherein the binding protein comprises:
(i) the amino acid sequence set forth in any one of SEQ ID NOs.:52, 53, 100, 101, 16, 17, 28, 29, 40, 41, 64, 65, 76, 77, 88, 89, 112, 113, 124, 125, 136, 137, 148, and 149, or a variant thereof comprising one, two, or three amino acid substitutions, wherein the amino acid sequence is optionally a CDR3β;
(ii) the amino acid sequence set forth in any one of SEQ ID NOs.: 51, 99, 15, 27, 39, 63, 75, 87, 111, 123, 135, and 147, or a variant thereof comprising one, two, or three amino acid substitutions, wherein the amino acid sequence is optionally a CDR2β;
(iii) the amino acid sequence set forth in any one of SEQ ID NOs.: 50, 98, 14, 26, 38, 62, 74, 86, 110, 122, 134, and 146, or a variant thereof comprising one, two, or three amino acid substitutions, wherein the amino acid sequence is optionally a CDR1β;
(iv) the amino acid sequence set forth in any one of SEQ ID NOs.: 57, 58, 105, 106, 21, 22, 33, 34, 45, 46, 69, 70, 81, 82, 93, 94, 117, 118, 129, 130, 141, 142, 153, and 154, or a variant thereof comprising one, two, or three amino acid substitutions, wherein the amino acid sequence is optionally a CDR3α;
(v) the amino acid sequence set forth in any one of SEQ ID NOs.: 56, 104, 20, 32, 44, 68, 80, 92, 116, 128, 140, and 152, or a variant thereof comprising one, two, or three amino acid substitutions, wherein the amino acid sequence is optionally a CDR2α; and/or
(vi) the amino acid sequence set forth in any one of SEQ ID NOs.: 55, 103, 19, 31, 43, 67, 79, 91, 115, 127, 139, and 151, or a variant thereof comprising one, two, or three amino acid substitutions, wherein the amino acid sequence is optionally a CDR1α.

13. The binding protein of claim 1, comprising:
(1) a TCR Vα comprising CDR1α, CDR2α, and CDR3α; and
(2) a TCR Vβ comprising CDR1β, CDR2β, and CDR3β,
wherein the CDR1α, CDR2α, CDR3α, CDR1β, CDR2β, and CDR3β are as set forth in:
(i) SEQ ID NOs.:55, 56, 57 or 58, 50, 51, and 52 or 53, respectively;
(ii) SEQ ID NOs.:103, 104, 105 or 106, 98, 99, and 100 or 101, respectively;
(iii) SEQ ID NOs.:19, 20, 21 or 22, 14, 15, and 16 or 17, respectively;
(iv) SEQ ID NOs.:31, 32, 33 or 34, 26, 27, and 28 or 29, respectively;
(v) SEQ ID NOs.:43, 44, 45 or 46, 38, 39, and 40 or 41, respectively;
(vi) SEQ ID NOs.:67, 68, 69 or 70, 62, 63, and 64 or 65, respectively;
(vii) SEQ ID NOs.:79, 80, 81 or 82, 74, 75, and 76 or 77, respectively;
(viii) SEQ ID NOs.:91, 92, 93 or 94, 86, 87, and 88 or 89, respectively;
(ix) SEQ ID NOs.:115, 116, 117 or 118, 110, 111, and 112 or 113, respectively;
(x) SEQ ID NOs.:127, 128, 129 or 130, 122, 123, and 124 or 125, respectively;
(xi) SEQ ID NOs.:139, 140, 141 or 142, 134, 135, and 136 or 137 respectively; or
(xii) SEQ ID NOs.:151, 152, 153 or 154, 146, 147, and 148 or 149, respectively.

14. The binding protein of claim 10, wherein the Vα comprises or consists of an amino acid sequence having at least 80%, at least 85%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.: 54, 102, 18, 30, 42, 66, 78, 90, 114, 126, 138, and 150, and/or wherein the Vβ comprises or consists of the amino acid sequence having at least 80%, at least 85%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.:49, 97, 13, 25, 37, 61, 73, 85, 109, 121, 133, and 145.

15. (canceled)

16. The binding protein of claim 10, comprising a Vα and a Vβ that comprise or consist of amino acid sequences having at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequences set forth in:
(i) SEQ ID NOs.:54 and 49, respectively;
(ii) SEQ ID NOs.:102 and 97, respectively;
(iii) SEQ ID NOs.:18 and 13, respectively;
(iv) SEQ ID NOs.:30 and 25, respectively;
(v) SEQ ID NOs.:42 and 37, respectively;
(vi) SEQ ID NOs.:66 and 61, respectively;
(vii) SEQ ID NOs.:78 and 73, respectively;
(viii) SEQ ID NOs.:90 and 85, respectively;
(ix) SEQ ID NOs.:114 and 109, respectively;
(x) SEQ ID NOs.:126 and 121, respectively;
(xi) SEQ ID NOs.:138 and 133 respectively; or
(xii) SEQ ID NOs.:150 and 145, respectively.

17.-20. (canceled)

21. The binding protein of claim 10, further comprising a TCR β-polypeptide constant domain (Cβ), a TCR α-polypeptide constant domain (Cα), or both, wherein, optionally,
(1) the Vβ and the Cβ together comprise a TCR β chain and/or the Vα and the Cα together comprise a TCR α chain, and/or
(2) the Cβ comprises or consists of an amino acid sequence having at least at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NO.:156 or 157, and/or the Cα comprises or consists of an amino acid sequence having at least at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NO.: 155; and/or
(3) the Cβ and/or the Cα comprises one or more non-native amino acid at a position such that when the Cβ and the Cα associate to form a dimer, a non-native disulfide bond is formed between the Cβ and the Cα, wherein, optionally, the non-native amino acid comprises a cysteine in the Cβ and/or a cysteine in the Cα; and/or
(4) the TCR Cβ comprises a cysteine amino acid at amino acid position 57, and wherein the TCR Cα comprises a cysteine amino acid at amino acid position 48.

22.-23. (canceled)

24. The binding protein of claim 1, wherein the binding protein comprises a TCR, a single-chain TCR (scTCR), a scTv, a chimeric antigen receptor (CAR), a TCR-mimic antibody or an antigen-binding fragment thereof, or any combination thereof.

25. The binding protein of claim 24, wherein binding protein comprises a TCR.

26.-29. (canceled)

30. An isolated polynucleotide encoding the binding protein of claim 1.

31. (canceled)

32. The polynucleotide of claim 30, wherein:
(1) the polynucleotide is codon-optimized for expression in a host cell, wherein, optionally, the host cell comprises an immune system cell, wherein, further optionally, the immune system cell comprises a T cell, a NK-T cell, or a NK cell; and/or
(2) the polynucleotide further comprises:
(i) a polynucleotide encoding a polypeptide that comprises an extracellular portion of a CD8 co-receptor α chain, wherein, optionally, the encoded polypeptide is or comprises a CD8 co-receptor α chain;
(ii) a polynucleotide encoding a polypeptide that comprises an extracellular portion of a CD8 co-receptor β chain, wherein, optionally, the encoded polypeptide is or comprises a CD8 co-receptor β chain; or
(iii) a polynucleotide of (i) and a polynucleotide of (ii).

33.-37. (canceled)

38. The polynucleotide of 3 claim 30, wherein the polynucleotide comprises DNA, RNA (optionally mRNA), or both, and/or wherein:
(1) the polynucleotide encodes an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.:48, 96, 12, 24, 36, 60, 72, 84, 108, 120, 132, and 144; and/or
(2) the polynucleotide comprises a polynucleotide having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprises or consists of, the nucleotide acid sequence set forth in any one of SEQ ID NOs.: 47, 95, 11, 23, 35, 47, 59, 71, 83, 107, 119, 131, and 143.

39.-40. (canceled)

41. A vector comprising the polynucleotide of claim 30.

42.-47. (canceled)

48. A host cell comprising the polynucleotide of claim 30, wherein the polynucleotide is optionally heterologous to the host cell.

49. The host cell of claim 48, wherein the host cell comprises a hematopoietic progenitor cell and/or an immune cell, optionally a human immune cell.

50. The host cell of claim 49, wherein the host cell comprises a T cell, a NK cell, a NK-T cell, a dendritic cell, a macrophage, a monocyte, a B cell, a plasma cell, or any combination thereof.

51. The host cell of claim 50, wherein the host cell comprises a CD4⁺ T cell, a CD8⁺ T cell, a CD4⁻ CD8⁻ double negative T cell, a γδ T cell, or any combination thereof, wherein, optionally, the host cell comprises a CD4⁺ T cell and a CD8⁺ T cell, wherein, further optionally, the CD4⁺ T cell, the CD8⁺ T cell, or both comprise (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

52.-69. (canceled)

70. A composition, comprising
the binding protein of claim 1, a polynucleotide encoding the binding protein of claim 1, a vector comprising a polynucleotide encoding the binding protein of claim 1, or a host cell comprising the binding protein of claim 1 and/or a polynucleotide encoding the binding protein of claim 1, and a pharmaceutically acceptable carrier, excipient, or diluent.

71. (canceled)

72. A method for treating a disease or disorder associated with expression of a SOX2 antigen comprising or consisting of the amino acid sequence according to any one of SEQ ID NOs:2-7, or with SOX2 expression or activity, in a subject, the method comprising administering to the subject an effective amount of
the host cell of claim 48 or a composition that comprises the host cell and a pharmaceutically acceptable carrier, excipient, or diluent
thereby treating the disease or condition.

73. A method for inducing an immune response in a subject having, and/or for treating in a subject, a disease or disorder associated with expression of a SOX2 antigen comprising or consisting of the amino acid sequence according to any one of SEQ ID NOs:2-7, or with SOX2 expression or activity, the method comprising administering to the subject an effective amount of T cells that express a TCR that specifically binds to a peptide:HLA complex expressed on the surface of a target cell, wherein the peptide comprises or consists of the amino acid sequence according to any one of SEQ ID NOs:2-7, and wherein the HLA is optionally HLA-A*02:01.

74.-75. (canceled)

76. The method of claim 72, wherein the subject is HLA-A*02:01⁺.

77. The method of claim 72, wherein the disease or condition is a cancer.

78. The method of claim 77, wherein the cancer comprises a hematological malignancy or a solid tumor.

79. The method of claim 77, wherein the cancer comprises multiple myeloma, plasma cell leukemia, ovarian cancer, glioma, lung cancer, neck cancer, cervical cancer, or any combination thereof.

80. The method of claim 72, wherein:
(i) the subject is human;
(ii) the subject has previously received one or more of: (1) surgery: (2) radiation therapy: (3) chemotherapy: (4) a hematopoietic stem cell transplant (HSC); and (5) an adoptive cell therapy, optionally comprising T cells expressing a CAR, and wherein the disease or disorder is optionally refractory to a prior therapy;
(iii) the method comprises administering to the subject an effective amount of the composition, wherein one or more of the host cells comprised in the composition is autologous to the subject; and/or
(iv) the method further comprises administering an inhibitor of an immune checkpoint molecule to the subject.

81.-95. (canceled)
